(19) United States
(12) Patent Application Publication
(10) Pub. No.: US 2010/0286136 A1
(43) Pub. Date: Nov. 11, 2010

(54) DIHYDRONAPHTHYRIDINYL AND RELATED COMPOUNDS FOR USE IN TREATING OPHTHALMOLOGICAL DISORDERS

(76) Inventors: Simon Jones, Harvard, MA (US); Dilara McCauley, Cambridge, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/775,577

(22) Filed: May 7, 2010

Related U.S. Application Data
(60) Provisional application No. 61/176,567, filed on May 8, 2009.

(51) Int. Cl. A61K 31/4375; A61K 31/538; A61K 31/506; A61K 31/4545; A61P 27/02; A61P 9/10; A61P 27/10; A61P 29/00

(52) U.S. Cl. 514/230.5; 514/256; 514/300

(57) ABSTRACT

The invention provides methods of using dihydronaphthyridinyl and related compounds to treat opthalmological disorders, such as, wet age-related macular degeneration, diabetic retinopathy, and high myopia. Pharmaceutical compositions and methods of synthesizing the dihydronaphthyridinyl and related compounds are provided.

### RELATED APPLICATIONS

This application claims the benefit of and priority to U.S. Provisional patent application Ser. No. 61/176,567 filed May 8, 2009, the contents of which are hereby incorporated by reference.

### FIELD

The invention generally relates to methods of using dihydronaphthyridinyl and related compounds to treat opthalmological disorders, such as, wet age-related macular degeneration, diabetic retinopathy, and high myopia.

### BACKGROUND

Opthalmological disorders affect millions of patients worldwide and resulting in impaired vision. The extent of vision loss varies with the type of opthalmological disorder and scope of available treatment options. Exemplary opthalmological disorders in need of new and better treatment options include diabetic retinopathy, age-related macular degeneration, choroidal neovascularization, and macular edema. Many of these disorders can cause a substantial loss in vision for the patient, and diabetic retinopathy, in particular, affects a substantial number of patients. For example, approximately eighty-percent of patients suffering from diabetes for ten years or more develop diabetic retinopathy.

Diabetic retinopathy is an eye disease that develops in patients suffering from diabetes due to changes in the cells that line blood vessels, i.e., the retinal microvascular endothelium. During diabetes mellitus, hyperglycemia can cause damage in a number of ways. For example, glucose, or a metabolite of glucose, binds to the amino groups of proteins, leading to tissue damage. In addition, excess glucose enters the polyol pathway resulting in accumulations of sorbitol. Sorbitol cannot be metabolized by cells of the retina and can contribute to high intracellular osmotic pressure, intracellular edema, impaired diffusion, tissue hypoxia, capillary cell damage, and capillary weakening. Diabetic retinopathy also involves thickening of capillary basement membranes which may in turn prevent pericytes, the predominant perivascular cell type in retinal capillaries, from contacting endothelial cells. Pericyte and endothelial cell death occurs through an apoptotic mechanism during diabetic retinopathy, where the loss of pericytes likely increases the permeability of the capillaries and leads to breakdown of the blood-retina barrier and blood flow dysregulation. Weakened capillaries lead to aneurysm formation and further leakage. These effects of hyperglycemia can also impair neuronal functions in the retina.

As the diabetes-induced microvascular pathology progress, retinal capillaries eventually become occluded and lead to multifocal areas of ischemia hypoxia within the retina. Hypoxic conditions in the non-perfused tissue elicits the production of growth factors capable of stimulating abnormal new blood vessel growth from existing vessels (angiogenesis). These pathologic new blood vessels grow into the vitreous and can cause loss of sight, a condition called proliferative diabetic retinopathy, since the new blood vessels are fragile and tend to leak blood into the eye. The proliferative type of diabetic retinopathy is characterized by neovascularization of the retina and optic disk which may project into the vitreous, proliferation of fibrous tissue, vitreous hemorrhage, and retinal detachment.

Age-related macular degeneration is another major cause of severe visual loss in the United States for individuals over the age of sixty. Age-related macular degeneration occurs in either a dry form or wet form. In the wet form of age-related macular degeneration, blood vessels grow from the choriocapillaris through defects in Bruch's membrane, and in some cases the underlying retinal pigment epithelium. Organization of serous or hemorrhagic exudates escaping from these vessels results in fibrovascular scarring of the macular region with attendant degeneration of the neuroretina, detachment and tears of the retinal pigment epithelium, vitreous hemorrhage and permanent loss of central vision.

Choroidal neovascularization involves the creation of new blood vessels in the choroid layer of the eye and leads to rapid deterioration of a patient's central vision. Current treatment options for choroidal neovascularization include laser treatment, but this therapy works for only a small percentage of the patient population. For example, even with successful conventional laser photocoagulation, the neovascularization recurs in about 50-70% of patients' eyes.

Macular edema is a common cause of severe visual impairment and can occur if the swelling, leaking, and hard exudates associated with background diabetic retinopathy occur within the macula, the central 5% of the retina most critical to vision. Background diabetic retinopathy typically consists of retinal microaneurisms that result from changes in the retinal microcirculation. These microaneurisms are usually the earliest visible change in retinopathy seen on exam with an opthalmoscope as scattered red spots in the retina where tiny, weakened blood vessels have ballooned out. The ocular findings in background diabetic retinopathy progress to cotton wool spots, intraretinal hemorrhages, leakage of fluid from the retinal capillaries, and retinal exudates. The increased vascular permeability is also related to elevated levels of local growth factors such as vascular endothelial growth factor. The macula is rich in cones, the nerve endings that detect color and upon which daytime vision depends. When increased retinal capillary permeability effects the macula, blurring occurs in the middle or just to the side of the central visual field. Vision loss may progress over a period of months.

Therefore, the need exists for new compositions and methods for treating opthalmological disorders, such as diabetic retinopathy, wet age-related macular degeneration, choroidal neovascularization, and macular edema. The present invention addresses this need and has other related advantages.

### SUMMARY

The invention provides dihydronaphthyridinyl and related compounds that are chemokine receptor modulators, e.g., antagonists, for use in treating opthalmological disorders. Accordingly, one aspect of the invention provides a method of treating a disorder selected from the group consisting of wet age-related macular degeneration, choroidal neovascularization, diabetic retinopathy, intraocular neovascularization, retinal vein occlusion, retinopathy of prematurity, angioid streak, high myopia, macular edema, ocular histoplasmosis, retinal detachment, retinitis pigmentosa, ischemic retinopathy, iris neovascularization, corneal neovascularization, retinal neovascularization, diabetic retinal ischemia, proliferative vitreoretinopathy, uveitis, iritis, inflammatory eye disease, and dry age-related macular degeneration. The method comprises administering to a patient in need thereof a therapeutically effective amount of a compound described herein, such as a compound of formula I-A.

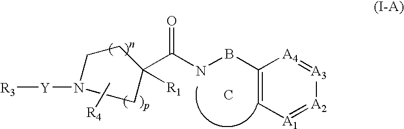

or a pharmaceutically acceptable salt thereof, wherein: the variables are as defined in the detailed description. In certain embodiments, the compound is of formula I-A1, I-A2, I-A3, I-A4, I-A5, I-A6, I-A7, I-A8, I-B1, I-B2, or I-C1.

Another aspect of the invention provides a pharmaceutical composition comprising a compound of formula I, e.g., formula I-A, I-A1, I-A2, I-A3, I-A4, I-A5, I-A6, I-A7, I-A8, I-B1, I-B2, or I-C1, and a pharmaceutically acceptable carrier

Another aspect of the invention provides for use of a compound described herein in the manufacture of a medicament for treating an opthalmological disorder, such as one or more of the opthalmological disorders described herein.

### DETAILED DESCRIPTION

The features and other details of the invention will now be more particularly described. It will be understood that particular embodiments described herein are shown by way of illustration and not as limitations of the invention. The principal features of this invention can be employed in various embodiments without departing from the scope of the invention. All parts and percentages are by weight unless otherwise specified. Further, if a variable is not accompanied by a definition, the previous definition of the variable controls.

### Therapeutic Applications

The invention provides dihydronaphthyridinyl and related compounds that are chemokine receptor modulators, e.g., antagonists, for use in treating opthalmological disorders. The dihydronaphthyridinyl and related compounds can be formulated in a pharmaceutical composition for administration to a patient, and preferably are antagonists of the chemokine CCR2 receptor. Through antagonism of the chemokine CCR2 receptor, the dihydronaphthyridinyl and related compounds minimize the affects of various monocyte chemotactic proteins, such as MCP-1 which is known to promote, for example, angiogenesis.

Chemokines are a group of 6-15 kDa inflammatory/immunomodulatory polypeptide factors that are released by a wide variety of cells such as macrophages, monocytes, eosinophils, neutrophiles, fibroblasts, vascular endothelial cells, smooth muscle cells, and mast cells, at inflammatory sites. Chemokines have the ability to stimulate directed cell migration, a process known as chemotaxis. Each chemokine contains four cysteine residues (C) and two internal disulfide bonds. Chemokines can be grouped into two subfamilies, based on whether the two amino terminal cysteine residues are immediately adjacent (“CC”) or separated by one amino acid (“CXC”). These differences correlate with the organization of the two subfamilies into separate gene clusters. The CC chemokines, such as RANTES, MIP-1a, MIP-1p, the monocyte chemotactic proteins (MCP-1, MCP-2, MCP-3, MCP-4, and MCP-5) and the eotaxins (-1 and -2) are chemotactic for, among other cell types, macrophages, T lymphocytes, eosinophils, dendritic cells, and basophils.

MCP-1 is a CC chemokine produced by monocytes/macrophages, smooth muscle cells, fibroblasts, and vascular endothelial cells. It causes cell migration and cell adhesion of monocytes, memory T lymphocytes, T lymphocytes and natural killer cells, as well as mediating histamine release by basophils. High expression of MCP-1 has been reported in diseases where accumulation of monocyte/macrophage and/or T cells is thought to be important in the initiation or progression of diseases, such as atherosclerosis and rheumatoid arthritis.

The published literature indicates that chemokines such as MCP-1 and MIP-1a attract monocytes and lymphocytes to disease sites and mediate their activation and thus are thought to be intimately involved in the initiation, progression and maintenance of diseases deeply involving monocytes and lymphocytes. The chemokines bind to specific cell-surface receptors belonging to the family of G protein-coupled seven-transmembrane-domain proteins which are termed “chemokine receptors.” On binding their cognate ligands, chemokine receptors transduce an intracellular signal through the associated trimeric G proteins, resulting in, among other responses, a rapid increase in intracellular calcium concentration, changes in cell shape, increased expression of cellular adhesion molecules, degranulation, and promotion of cell migration.

Genes encoding receptors of specific chemokines have been cloned, and it is now known that these receptors are G protein-coupled seven-transmembrane receptors present on various leukocyte populations. So far, at least eleven CC chemokine receptors (CCR1-CCR11) have been identified. For example, MIP-1a is a ligand for CCR1 and CCR5, and MCP-1 is a ligand for CCR2A and CCR2B.

CCR2 (also termed CKR-2, MCP-1RA or MC1RB) is predominantly expressed on monocytes and macrophages, and is necessary for macrophage-dependent inflammation. CCR2 is a G protein-coupled receptor (GPCR) which binds with high affinity (Kd of 1 nM) to several members of the MCP family of chemokines (CCL2, CCL7, CCL8, etc.), eliciting a chemotactic signal that results in directed migration of the receptor-bearing cells.

The scientific literature supports the use of chemokine CCR2 receptor antagonists in treating various opthalmological disorders. In particular, various scientific reports illustrate a link between neovascularization and the MCP-1 protein. Because MCP-1 is a ligand for the chemokine CCR2 receptor, compounds that antagonize the chemokine CCR2 receptor are a promising therapeutic for opthalmological disorders associated with neovascularization of ocular tissue, such as choroidal neovascularization associated with wet age-related macular degeneration, angiod streaks, high myopia, ocular histoplasmosis, diabetic retinopathy, and branch retinal vein occlusion.

For instance, Yoshida and coworkers investigated the role of MCP-1 and macrophage inflammatory protein-1α (MIP-1α) in a mouse model of ischemic retinopathy. Yoshida et al., J. Leukoc. Biol., 2003, 73:137-144. Their findings indicate that MCP-1 and MIP-1α are involved in inducing retinal neovascularization and play a role in the inflammation induced by ischemic retinopathy. Retinal neovascularization is one form of intraocular neovascularization, which is a major cause of decreased vision in patients with proliferative diabetic retinopathy, retinal vein occlusion and retinopathy of prematurity. Coadministration of neutralizing antibodies for MCP-1 and MIP-1a were reported to inhibit retinal neovascularization by thirty percent. These findings suggest that compounds that modulate the effects of MCP-1 may be useful in treating intraocular neovascularization, and diseases resulting therefrom, such as proliferative diabetic retinopathy, retinal vein occlusion, and retinopathy of prematurity.

The results of a scientific study conducted by Tsutsumi and coworkers suggest that compounds that modulate the effects of MCP-1 may offer therapeutic potential in treating choroidal neovascularization, such as choroidal neovascularization associated with age-related macular degeneration, angiod streaks, high myopia, ocular histoplasmosis, diabetic retinopathy, and branch retinal vein occlusion. See Tsutsumi et al., J. Leukoc. Biol., 2003, 74:25-32. In particular, Tsutsumi and coworkers studied the mechanisms by which macrophages participate in the formation of choroidal neovascularization. The studies utilized CCR2 knockout (KO) mice because MCP-1 (the main ligand of CCR2) is strongly associated with macrophage migration—in fact, previous studies have shown that macrophages were not recruited to inflammatory lesions in CCR2 knockout mice. The studies by Tsutsumi and coworkers showed that the number of new blood vessels in CCR2 knockout (KO) mice was significantly fewer than in control mice.

The results of a scientific study by Yamada and coworkers showed that a therapy for choroidal neovascularization (CNV) resulted in lower levels of the MCP-1 protein. Yamada et al., Invest. Opthalmol. Vis. Sci., 2007, 48:1839-1843. The study investigated the effect of atorvastatin on experimental CNV. The scientific report suggests that atorvastatin inhibits CNV by suppressing macrophage infiltration into the retinal pigment epithelium/choroid. The authors also explain that the mean level of CCL2/MCP-1 protein was significantly lower in patients receiving atorvastatin. Because recruitment of monocytes occurs during the early stages of inflammatory and angiogenic processes, and MCP-1 is involved in monocyte recruitment, the results provide reason to believe that therapeutic agents capable of modulating the effects of MCP-1 could help treat choroidal neovascularization.

In addition to the foregoing, several scientific reports illustrate a link between the presence of macrophages and choroidal neovascularization. This discovery is an important step in understanding the potential mechanism(s) by which CCR2 antagonists may be able to treat choroidal neovascularization because macrophages are recruited by MCP-1 protein, and CCR2 antagonists modulate the activity of the receptor for the MCP-1 protein.

For instance, Bainbridge explains that macrophages express proangiogenic cytokines, which lead to choroidal neovascularization. Bainbridge, Expert Rev. Opthalmol., 2007, 2:981-986. Bainbridge further explains that controlling the development of choroidal neovascularization is believed to depend at least in part on macrophage interaction with the retinal pigment epithelium. As noted above, because MCP-1 can recruit macrophages, compounds that modulate the effects of MCP-1 may offer means to minimize macrophage recruitment and provide a therapeutic benefit in treating choroidal neovascularization.

Scientific reports by Espinosa-Heidmann, Sakurai, and Izumi-Nagai further illustrate the role that macrophages are believed to have in the development of choroidal neovascularization (CNV). Espinosa-Heidmann and co-workers investigated whether treatment with clondrate liposomes (CL₂MDP-lip) diminishes the severity of neovascularization in a mouse model of CNV. Espinosa-Heidmann et al., Invest. Opthalmol. Vis. Sci., 2003, 44:3586-3592. They concluded that macrophage depletion using CL₂MDP-lip reduced the size, cellularity and vascularity of CNV, thereby supporting the hypothesis that macrophages contribute to the severity of CNV lesions. Sakuri and coworkers studied the role of macrophages in the development of laser-induced CNV by selective depletion with CL₂MDP-lip. Sakurai et al., Invest. Opthalmol. Vis. Sci., 2003, 44:3578-3585. They found that macrophage depletion reduced the size and leakage of laser-induced CNV and was associated with decreased macrophage infiltration and VEGF protein. Izumi-Nagai and coworkers investigated the role of interleukin IL-6 in the development of laser-induced CNV. Izumi-Nagai et al., Am. J. Pathol., 2007 170:2149-2158. They found that IL-6 receptor neutralization led to significant inhibition of inflammation-related molecules, such as MCP-1, intracellular adhesion molecule-1, VEGF, and of macrophage infiltration into CNV.

The scientific literature also supports the use of chemokine CCR2 receptor antagonists in treating diabetic retinopathy. In particular, various scientific reports illustrate a link between diabetic retinopathy and the MCP-1 protein. For example, in a study on inflammatory and angiogenic factors involved in the development of diabetic retinopathy in diabetic patients, Maier and coworkers observed a significant increase in vitreous levels of IP-10, MCP-1, and VEGF, which suggests that these factors are involved in the development diabetic retinopathy. Maier et al., Molecular Vision, 2008, 14:637-643. As such, therapeutic agents that modulate the activity of the receptor for MCP-1 may offer a therapeutic benefit in the prophylaxis and/or treatment of diabetic retinopathy.

Further, scientific reports illustrate a link between angiogenesis and the MCP-1 protein. The angiogenesis-promoting effects of the MCP-1 protein implicates this protein in various opthalmological disorders associated with angiogenesis of ocular tissue, such as wet age-related macular degeneration. The connection between MCP-1 and angiogenesis of ocular tissue also suggests that compounds capable of inhibiting the activity of MCP-1, such as by antagonizing the receptor for MCP-1, may have therapeutic potential in treating diseases associated with angiogenesis of ocular tissue.

For instance, a recent report by Ma and coworkers describes the role of MCP-1 on the angiogenic effect of TGF-β. Ma et al., Blood, 2007, 109:987-994. Their findings suggest that MCP-1 mediates TGF-β-stimulated angiogenesis by enhancing migration of mural cells towards endothelial cells and thereby promoting maturation of new blood vessels. This TGF-β-promoted formation of new blood vessels was attenuated when MCP-1 activity was blocked by addition of an antibody for MCP-1.

The mechanism by which MCP-1 induces angiogenesis was studied by Hong and coworkers. Hong et al., Blood, 2005, 105(4): 1405-1407. Their results suggest that MCP-1 induced angiogenesis is a two-step process involving the induction of VEGF-A gene expression by MCP-1 and the subsequent VEGF-A-induced angiogenesis. This angiogenesis may affect the growth of tumors, inflammatory lesions such as artheromatous plaques, and arterio-occlusive diseases. The study performed by Hong adds to the information contained in earlier reports by Niyama and Parenti. Niyama and coworkers investigated the role of MCP-1 in ischemia-induced neovascularization. Niyama et al., J. Am. Coll. Cardiol., 2004, 44:661-666. Their results indicate that MCP-1 participated in angiogenesis and arteriogenesis, two types of neovascularization. Parenti and coworkers studied the role of MCP-1 on vascular smooth muscle cell proliferation and migration. Parenti et al., Am. J. Physiol. Heart Circ. Physiol., 2004, 286:H1978-H1984. They concluded that MCP-1 directly promotes vascular smooth muscle cell proliferation through the autocrine production of vascular endothelial growth factor-A (VEGF-A).

The scientific literature also supports the use of chemokine CCR2 receptor antagonists in treating opthalmological disorders associated with photoreceptor apoptosis. Photoreceptor apoptosis can lead to vision loss and is associated with several opthalmological disorders, including macular degeneration, retinal detachment, diabetic retinopathy and retinopathy of prematurity. See, for example, Nakazawa et al., PNAS, 2007, 104:2425-2430. Nakazawa and coworkers performed a study evaluating the role of MCP-1 in mediating photoreceptor apoptosis in an experimental model of retinal detachment. Id. Results from the study indicated that MCP-1 plays a critical role in mediating photoreceptor apoptosis in retinal detachment. Moreover, a MCP-1 blocking antibody reduced the amount of photoreceptor apoptosis induced by retinal detachment. These results suggest that compounds capable of inhibiting the activity of MCP-1, such as by antagonizing the receptor for MCP-1, may have therapeutic potential in treating photoreceptor apoptosis induced by MCP-1 and/or opthalmological disorders associated with photoreceptor apoptosis induced by MCP-1.

Accordingly, one aspect of the invention provides a method of treating a disorder selected from the group consisting of wet age-related macular degeneration, choroidal neovascularization, diabetic retinopathy (including proliferative diabetic retinopathy), intraocular neovascularization, retinal vein occlusion (including branch retinal vein occlusion and central retinal vein occlusion), retinopathy of prematurity, angioid streak, high myopia, macular edema, ocular histoplasmosis, retinal detachment, retinitis pigmentosa, ischemic retinopathy, iris neovascularization, corneal neovascularization, retinal neovascularization, diabetic retinal ischemia, proliferative vitreoretinopathy, uveitis, iritis, inflammatory eye disease, and dry age-related macular degeneration. The method comprises administering to a patient in need thereof a therapeutically effective amount of a compound described herein, such as a compound of formula I-A:

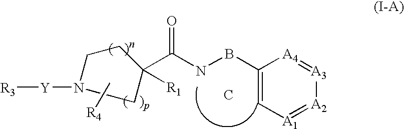

or a pharmaceutically acceptable salt thereof, wherein:

R₁ is hydrogen; alkyl, alkoxyalkyl, alkoxyphenyl, alkylthioalkyl, alkylamino, —SO₂(alkyl), C₃₋₆ cycloalkyl, C₃₋₆ heterocycloalkyl, aryl, heteroaryl, aralkyl, or heteroaralkyl, each of which is optionally substituted with 1, 2, or 3 R₅ substituents; or R₁ is optionally substituted (C₁-C₆alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 R₅ substituents;

Y is a direct bond or is CO, SO₂, —N(H)CO, —N(H)SO₂, C(═NH), C₁₋₄ alkylene, C₂₋₄ alkenylene, C₂₋₄ alkynylene, C₃₋₆ cycloalkylene, arylene, heterocycloalkylene, heteroarylene, —C(O)alkylene, —N(H)C(O)alkylene, or —O-alkylene; each of which may be optionally substituted with 1, 2, or 3 R₅ substituents;

R₃ is hydrogen; alkyl, alkenyl, alkynyl, cycloalkyl, aryl, heterocycloalkyl, heteroaryl, or —N(R₆)(R₇); each of which is optionally substituted with 1, 2, or 3 R₅ substituents; or R₃ is

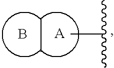

which is an optionally substituted fused aromatic or partially aromatic bicyclic or tricyclic ring, containing at least one nitrogen atom;

R₄ is hydrogen; halo; C₁₋₈ alkyl, alkenyl, or alkynyl optionally interrupted by oxygen or sulfur; cycloalkyl; alkoxy; arylalkoxy; or heteroarylalkoxy;

R₅, when present, represents independently for each occurrence hydrogen, halo, hydroxy, alkyl, alkenyl, cycloalkyl, alkoxy, —CO₂H, —CO₂C₁₋₃alkyl, cyano, aryl, heteroaryl, aralkyl, heteroaralkyl, oxo, —CF₃, —O—CF₃, —O—CHF₂, —O—CH₂F, —O-aryl, —N(H)alkyl, —N(H)SO₂-alkyl, —N(H)C(O)alkyl, —SO₂N(H)alkyl, —SO₂N(alkyl)C(O)alkyl, or —C(O)N(H)SO₂alkyl;

R₆ is hydrogen, alkyl, cycloalkyl, heterocycloalkyl, aryl, or heteroaryl;

R₇ is hydrogen or C₁₋₃ alkyl;

n is 0, 1, 2, or 3;

p is 1 or 2;

A₁, A₂, A₃, and A₄ are independently N or C—R₅, provided that at least two of A₁, A₂, A₃, or A₄ are C—R₅; and

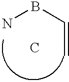

is an optionally substituted 5, 6, or 7-membered mono- or bicyclic ring optionally containing a heteroatom selected from the group consisting of O, S, SO, SO₂, N—H, N-alkyl, and N—CO-alkyl, in which B is C₁-C₂alkylene or C₂-C₄alkenylene, and in which the ring is optionally substituted with 1 or 2 halo, methyl, or ethyl groups, or is geminally substituted to form a cyclopropyl ring.

In certain embodiments, R₁ is hydrogen. In certain other embodiments, R₁ is alkyl. In certain other embodiments, R₁ is alkoxyalkyl, alkoxy-CHF₂, alkoxy-CH₂F, alkoxy-CF₃, C₃₋₆ cycloalkyl, C₃₋₆ heterocycloalkyl, aryl, heteroaryl, or (C₁-C₆alkylene)-R₁ₐ, wherein R₁ₐ is C₃₋₆ cycloalkyl, C₃₋₆ heterocycloalkyl, aryl, or heteroaryl, each of which may be independently optionally substituted with 1, 2, or 3 R₅ substituents. In certain other embodiments, R1 alkoxyalkyl. Further, exemplary R₁ moieties include —CH₂—O—CH₃, —CH₂—O—CF₃, —CH₂—O—CHF₂, —CH₂—O—CH₂F, —CH₂—O—CH₂—CH₃, —CH₂—O—CH—(CH₃)₂, and —CH₂—CN.

In certain other embodiments, R₁ is

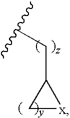

wherein z is 1, 2, or 3; y is 1, 2, 3, or 4; and x is O, NH, CH₂, CF₂, or N(C₁₋₈alkyl).

In certain other embodiments, R₁ is methyl;

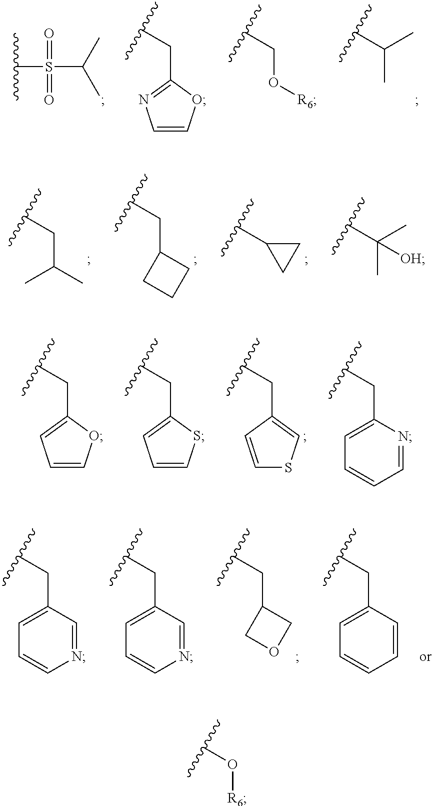

any of which may be optionally substituted on carbon with 1, 2, or 3 R₅, and wherein R₆ is hydrogen, alkyl, cycloalkyl, heterocycloalkyl, aryl, heteroaryl, or SO₂R₈; and R₈ is an alkyl, alicyclic, aryl, heterocyclic, or heteroaryl group. In certain other embodiments, R₁ is

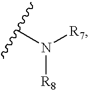

wherein R₇ and R₈ can be taken together with the nitrogen to which they are attached to form an 3, 4, 5, or 6-membered ring which itself may be optionally substituted with 1, 2, or 3, R₅; or R₇ is hydrogen, or C₁₋₃ alkyl; and R₈ is an alkyl, alicyclic, aryl, heterocyclic, or heteroaryl group.

In certain embodiments, Y is CH₂. In certain other embodiments, Y is

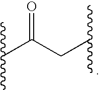

In certain other embodiments, Y is

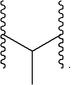

In certain other embodiments, Y is

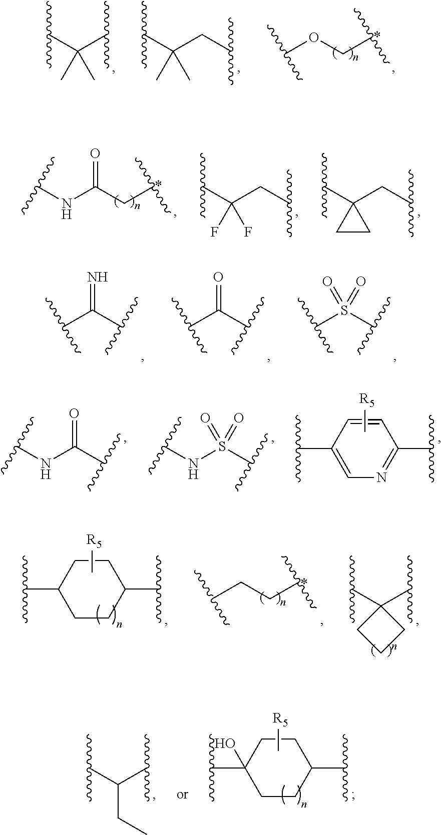

wherein n is 0, 1 or 2.

In certain other embodiments, R₃ is

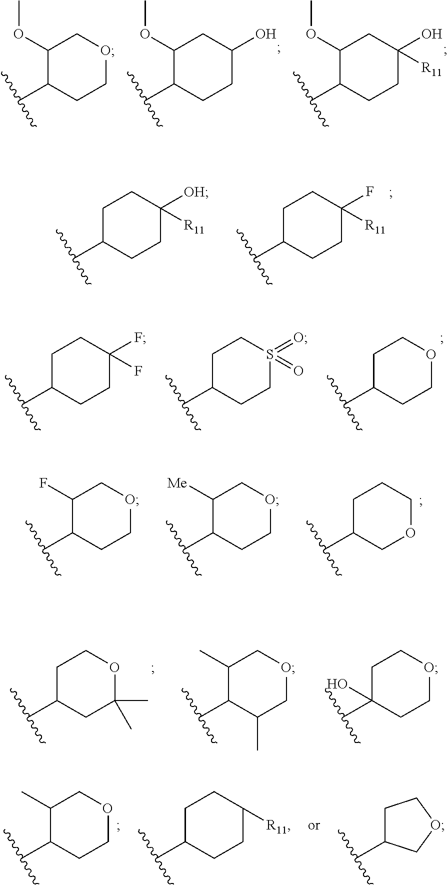

wherein R₁₁ is hydrogen or is C₁₋₆ alkyl, (C₁-C₆alkylene)cycloalkyl, aralkyl, or heteroaralkyl, any of which may be optionally substituted with halo, hydroxy, alkyl, alkenyl, cycloalkyl, C₁₋₃alkoxy, —CO₂H, —CO₂C₁₋₃alkyl, cyano, aryl, heteroaryl, —CF₃, —O—CF₃, —O—CH₂F, or —O—CHF₂.

In certain other embodiments, R₃ is

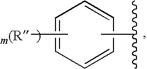

wherein m is 1, 2, or 3; and R″ represents independently for each occurrence hydroxyl, halo, alkoxy, halo-alkoxy, C₁₋₃ alkyl-S(O)₂—NH—, —CO₂H, C₁₋₃ alkyl-C(O)—NH—, alkyl-SO₂NHCO—, aryl, halo-substituted aryl, or heteroaryl; or wherein two R″ attached to adjacent carbon atoms are taken together to form

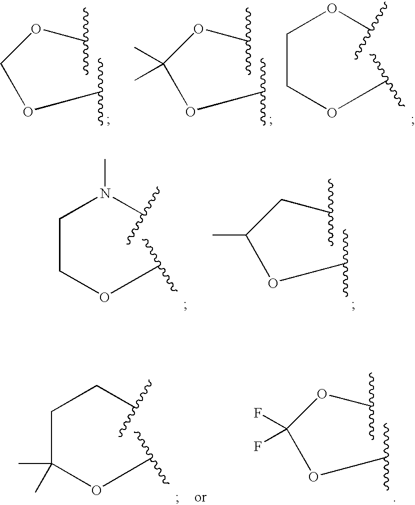

In certain other embodiments, R₃ is

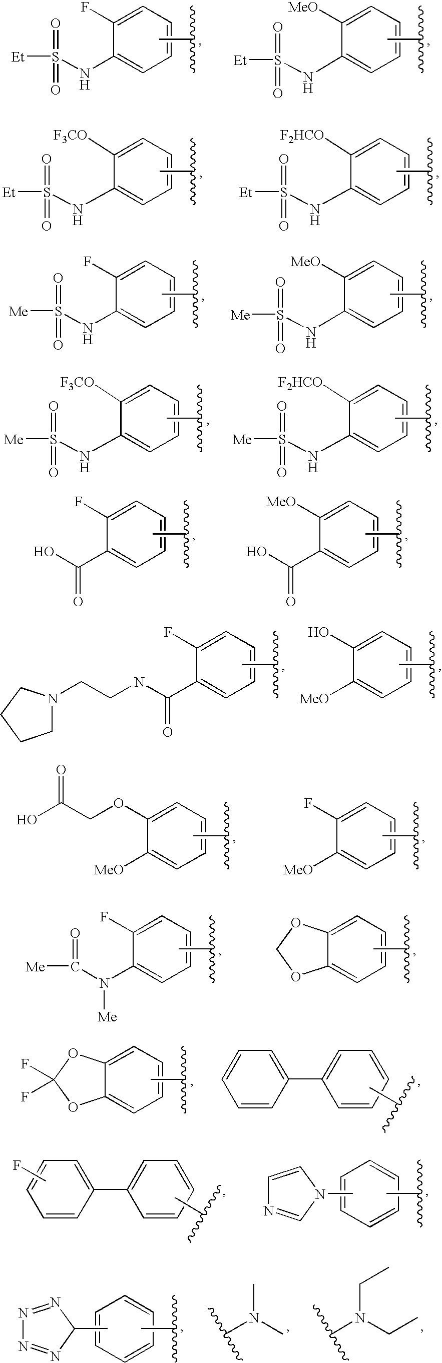

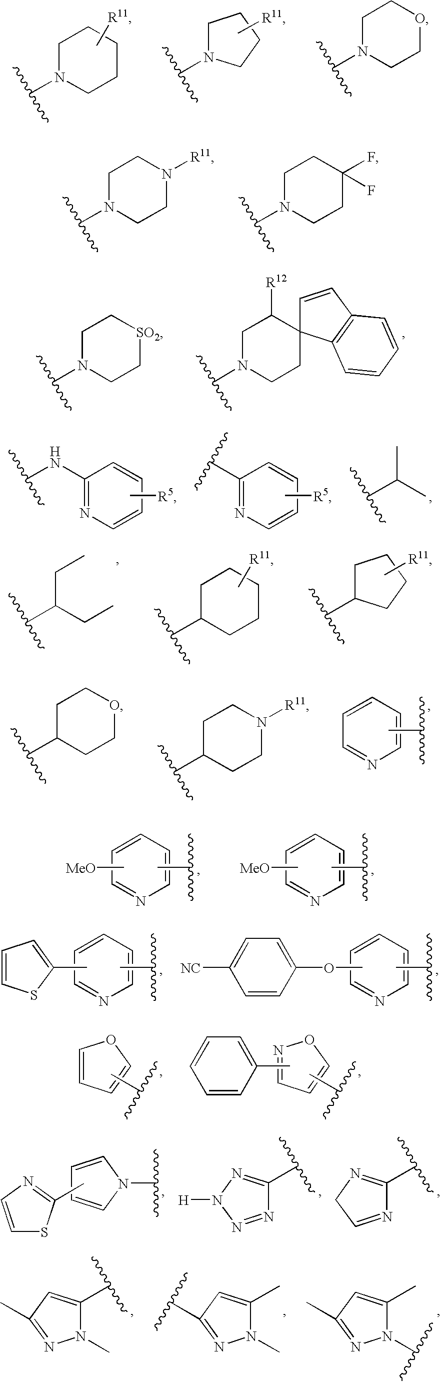

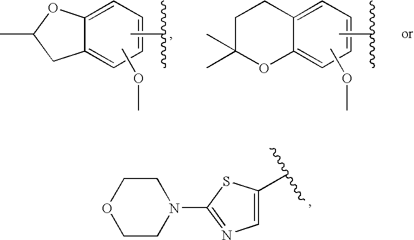

wherein R¹¹ is hydrogen; lower alkyl; hydroxy; amino; alkoxy; SO₂-lower alkyl; or an unsubstituted alicyclic, aromatic, heterocyclic or heteroaromatic ring; and R¹² is hydrogen or C₁₋₃ alkyl.

In certain embodiments, R₃ is

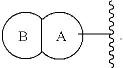

In certain embodiments,

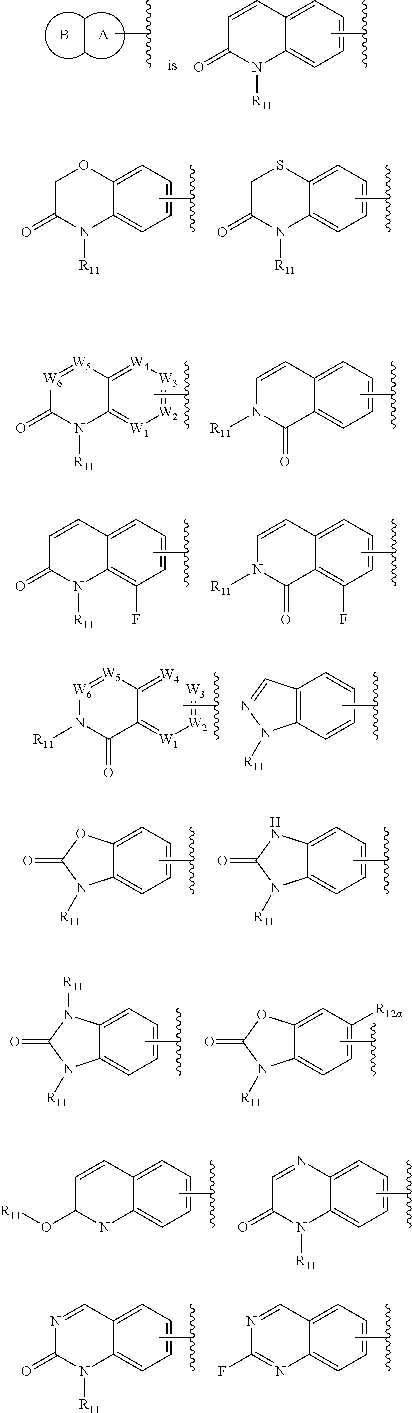

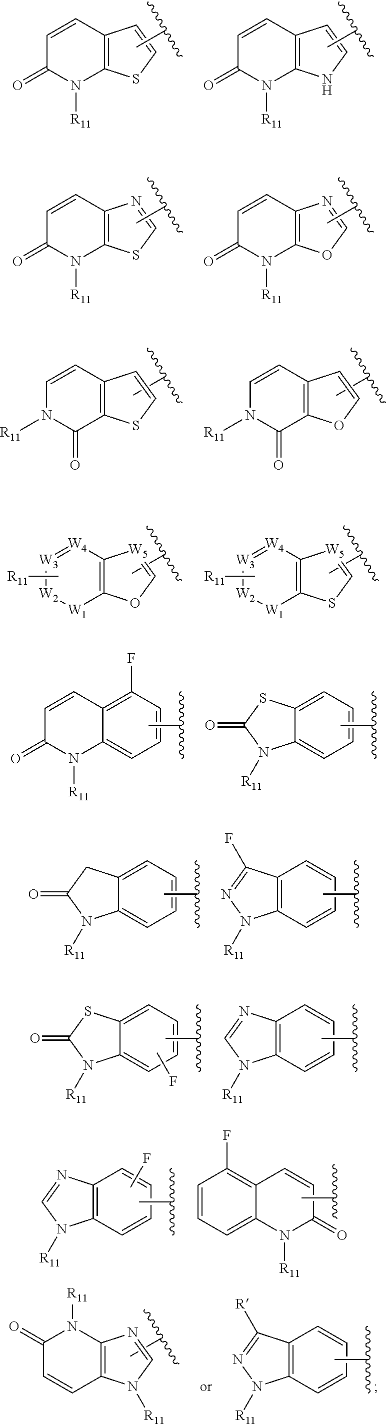

wherein W₁, W₂, W₃, W₄, W₅, and W₆ are independently C, N, CO, ═C—OH, C—OR₁₀ or C—R₁₀; R₁₀ is hydrogen, C₁₋₆ alkyl, C₁₋₅ alkylthio, C₁₋₅ alkoxy, halogen, hydroxyl, cyano, halogen-substituted C₁₋₆ alkyl, or halogen-substituted C₁₋₅ alkoxy; R₁₁ is hydrogen or is C₁₋₆ alkyl, (C₁-C₆alkylene)cycloalkyl, aralkyl, or heteroaralkyl, any of which may be optionally substituted with halo, hydroxy, alkyl, alkenyl, cycloalkyl, C₁₋₃alkoxy, —CO₂H, —CO₂C₁₋₃alkyl, cyano, aryl, heteroaryl, —CF₃, —O—CF₃, —O—CH₂F, or —O—CHF₂; R₁₂ₐ is H, halo, alkoxy, or alkyl; and R′ is alkyl, haloalkyl, or cycloalkyl.

In certain embodiments,

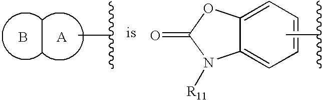

wherein R₁₁ is hydrogen, methyl, ethyl, or propyl.

In certain embodiments, R₅ is fluoro. In certain other embodiments, R₅ is chloro. In certain other embodiments, R₅ include methyl, ethyl, methoxy, ethoxy, trifluoromethyl, or trifluoromethoxy. When two R₅ groups are attached to the same carbon, they may be taken together to form a 3, 4, 5, or 6-membered ring or an oxo (i.e., C═O) group.

In certain embodiments,

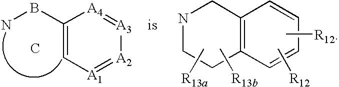

In certain other embodiments,

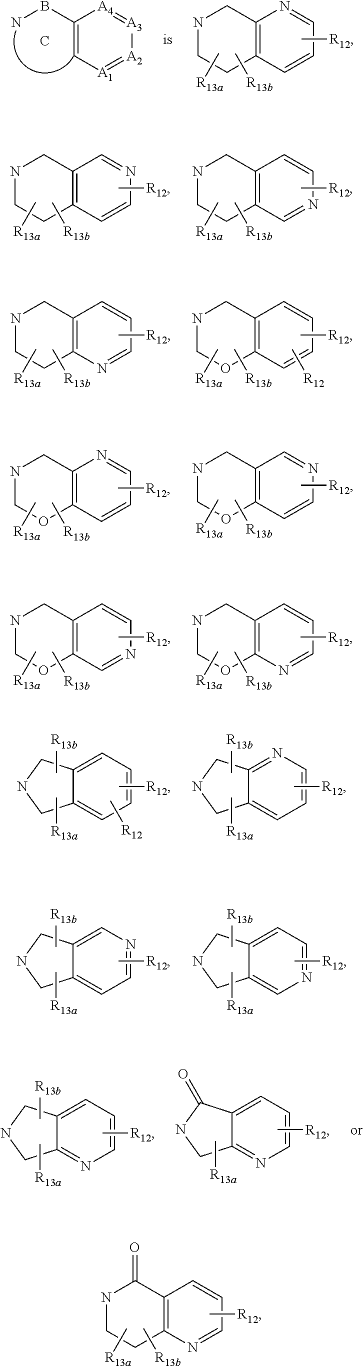

wherein R₁₂ independently for each occurrence is hydrogen, halo, alkyl, haloalkyl, haloalkoxy, alkoxy, or cyano; and

R₁₃ₐ and R_{13b} are each independently hydrogen, halo, alkyl, haloalkyl, alkoxy, haloalkoxy, or if R₁₃ₐ and R_{13b} are attached to the same carbon, they can form C═O when taken together with the carbon to which they are attached.

In certain embodiments, R₁₂ is hydrogen, halo, alkyl, or haloalkyl. In certain other embodiments, R₁₂ is fluoro, chloro, methyl, ethyl, or trifluoromethyl.

In certain embodiments, R₁₃ₐ and R_{13b} are each independently hydrogen, halo, alkyl, haloalkyl, alkoxy, or haloalkoxy. In certain other embodiments, R₁₃ₐ and R_{13b} each independently are fluoro, chloro, methyl, ethyl, trifluoromethyl, methoxy, or trifluoromethoxy.

In certain embodiments,

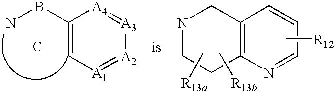

and R₁₂, R₁₃ₐ, and R_{13b} are each independently hydrogen, halo, alkyl, or haloalkyl.

In certain embodiments, R₄ is hydrogen.

In certain embodiments, n and p are 1. In certain other embodiments, n is 1. In certain other embodiments, n is 2. In certain embodiments, p is 1. In certain other embodiments, p is 2. In certain embodiments, p is 1, and n is 1 or 2. In certain other embodiments, p is 2 and n is 1.

In certain embodiments, the compound is a compound of formula I-A1:

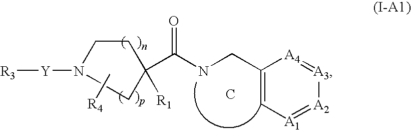

wherein the variables are as defined above for formula I-A.

In certain embodiments, the compound is a compound of formula I-A2:

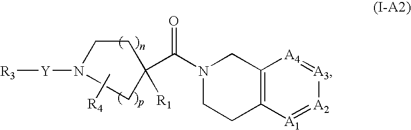

wherein the variables are as defined above for formula I-A.

In certain embodiments, the compound is a compound of formula I-A3:

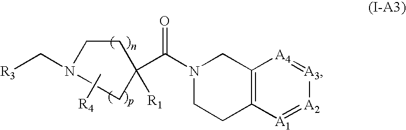

wherein the variables are as defined above for formula I-A.

In certain embodiments, the compound is a compound of formula I-A4:

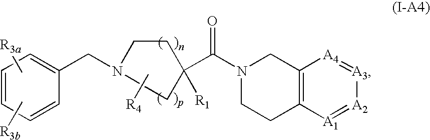

wherein R₃ₐ and R_{3b} are each independently hydrogen, halo, hydroxy, lower alkyl, lower alkenyl, cycloalkyl, C₁₋₃ alkoxy, cyano, or CF₃, or R₃ₐ and R_{3b} taken together form

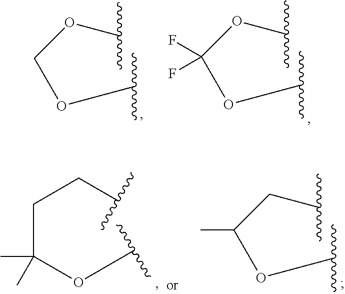

and the remainder of the variables are as defined above for formula I-A.

In certain embodiments, the compound is a compound of formula I-A5:

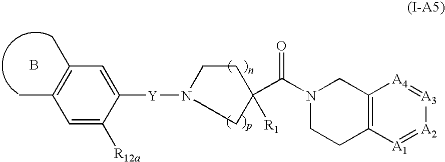

wherein

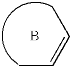

is an aromatic or unsaturated ring which may be optionally substituted with 1 or 2 groups selected from halo, hydroxy, alkyl, alkenyl, cycloalkyl, C₁₋₃alkoxy, —CO₂H, —CO₂C₁₋₃alkyl, cyano, aryl, heteroaryl, —CF₃, —O—CF₃, —O—CH₂F, —O—CHF₂, —N(H)alkyl, —N(H)SO₂-alkyl, —N(H)C(O)alkyl, or —SO₂N(H)alkyl; R₁₂ₐ is selected from the group consisting of H, halo, alkyl, or alkoxy; and the remainder of the variables are as defined above for formula I-A.

In certain embodiments, the compound is a compound of formula I-A6:

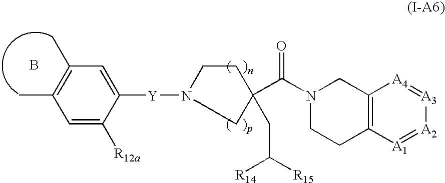

wherein R₁₄ and R₁₅ are each independently optionally substituted alkyl or taken together with the carbons to which they are attached form a 3, 4, 5, or 6-membered ring optionally containing one heteroatom selected from the group consisting of O, S, NH, and N-alkyl, which ring is optionally substituted with 1, 2, or 3 groups selected from the group consisting of halo, alkyl, alkoxy, and haloalkoxy; R₁₂ₐ is selected from the group consisting of H, halo, alkyl, or alkoxy; and the remainder of the variables are as defined above for formula I-A.

In certain embodiments, the compound is a compound of formula I-A7:

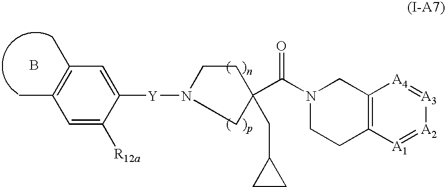

where R₁₂ₐ is selected from the group consisting of H, halo, alkyl, or alkoxy; and the remainder of the variables are as defined above for formula I-A.

In certain embodiments, the compound is a compound of formula I-A8:

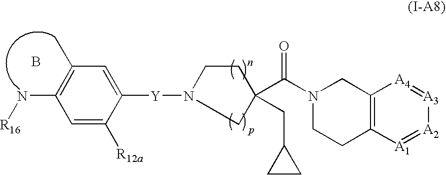

wherein R₁₆ is H, or is alkyl, cycloalkyl, (C₁-C₆alkylene)cycloalkyl, aralkyl, heteroaralkyl, any of which may be optionally substituted with halo, hydroxy, alkyl, alkenyl, cycloalkyl, C₁₋₃alkoxy, —CO₂H, —CO₂C₁₋₃alkyl, cyano, aryl, heteroaryl, —CF₃, —O—CF₃, —O—CH₂F, or —O—CHF₂; R₁₂ₐ is selected from the group consisting of H, halo, alkyl, or alkoxy; and the remainder of the variables are as defined above for formula I-A.

In certain embodiments, the invention provides one of the aforementioned compounds, wherein p is 1. In certain other embodiments, p is 2. In certain other embodiments, p is 2 and n is 1.

In certain embodiments, the compound is a compound of formula I-B1:

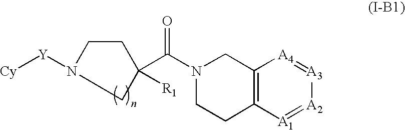

wherein Cy is an unsubstituted cyclic or bicyclic ring optionally having partial aromaticity and optionally having one or more heteroatoms; or pharmaceutically acceptable salts thereof; Y may be a direct bond or alkyl; and the remainder of the variables are as defined above for formula I-A. Specific values for Cy include

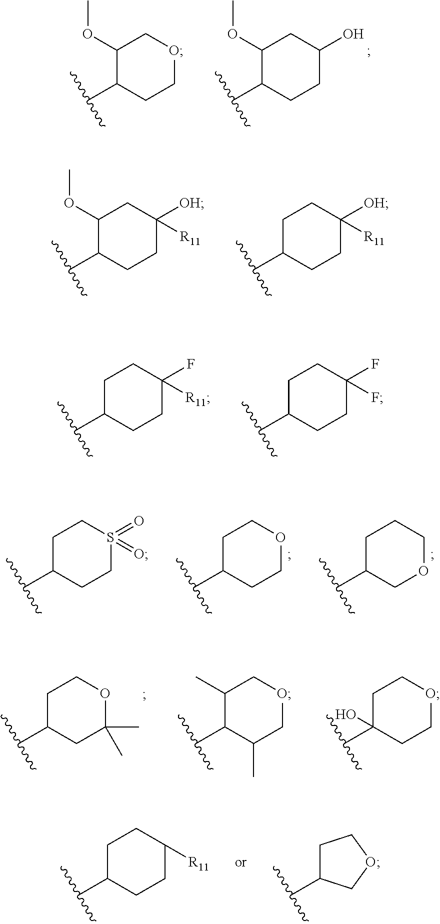

wherein R₁₁ is hydrogen or is C₁₋₆ alkyl, (C₁-C₆alkylene)cycloalkyl, aralkyl, or heteroaralkyl, any of which may be optionally substituted with halo, hydroxy, alkyl, alkenyl, cycloalkyl, C₁₋₃alkoxy, —CO₂H, —CO₂C₁₋₃alkyl, cyano, aryl, heteroaryl, —CF₃, —O—CF₃, —O—CH₂F, or —O—CHF₂.

In certain embodiments, the compound is a compound of formula I-B2:

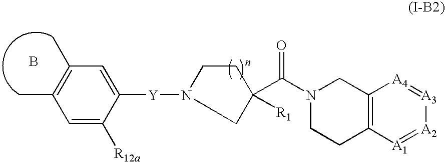

or a pharmaceutically acceptable salt thereof, wherein

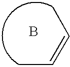

is an unsaturated heterocyclic ring optionally substituted with 1 or 2 groups selected from the group consisting of halo, alkyl, and oxo; Y is C₁-C₃ alkylene; and R₁ is alkoxyalkyl or alkyl; and R₁₂ₐ is selected from the group consisting of H, halo, alkyl, or alkoxy; and the remainder of the variables are as defined above for formula I-A. In certain embodiments, A₁, A₂, A₃, and A₄ are independently N or C—R₅, provided that at least two of A₁, A₂, A₃, or A₄ are C—R₅; R₁ is alkoxyalkyl; R₅ represents independently for each occurrence hydrogen, halo, hydroxy, alkyl, alkenyl, cycloalkyl, alkoxy, cyano, or —CF₃; R₁₂ₐ is hydrogen, halo, alkyl, or alkoxy; n is 1 or 2; Y is C₁-C₃ alkylene; and

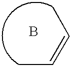

is an unsaturated heterocyclic ring optionally substituted with 1 or 2 groups selected from the group consisting of halo, alkyl, and oxo.

In certain embodiments, the compound is a compound of formula I-B3:

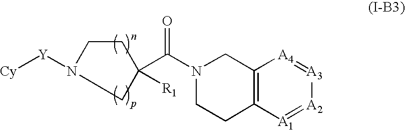

wherein Cy is an unsubstituted cyclic or bicyclic ring optionally having partial aromaticity and optionally having one or more heteroatoms; or pharmaceutically acceptable salts thereof; Y may be a direct bond or alkyl; and the remainder of the variables are as defined above for formula I-A. Specific values for Cy include

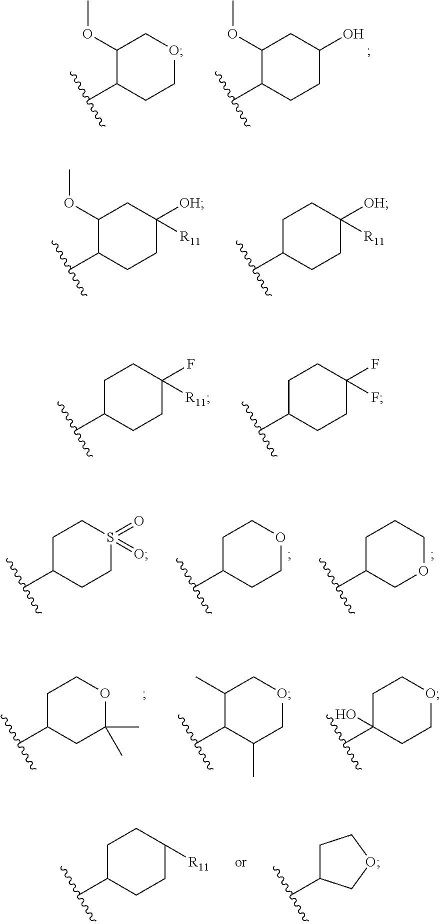

wherein R₁₁ is hydrogen or is C₁₋₆ alkyl, (C₁-C₆alkylene)cycloalkyl, aralkyl, or heteroaralkyl, any of which may be optionally substituted with halo, hydroxy, alkyl, alkenyl, cycloalkyl, C₁₋₃alkoxy, —CO₂H, —CO₂C₁₋₃alkyl, cyano, aryl, heteroaryl, —CF₃, —O—CF₃, —O—CH₂F, or —O—CHF₂.

In certain embodiments, the compound is a compound of formula I-B4:

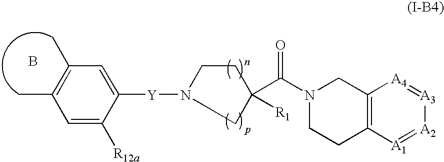

wherein

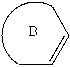

is an unsaturated heterocyclic ring optionally substituted with 1 or 2 groups selected from the group consisting of halo, alkyl, and oxo; Y is C₁-C₃ alkylene; and R₁ is alkoxyalkyl or alkyl; R₁₂ₐ is selected from the group consisting of H, halo, alkyl, or alkoxy; and the remainder of the variables are as defined above for formula I-A.

In certain embodiments, the invention provides one of the aforementioned compounds, wherein p is 1. In certain other embodiments, p is 2. In certain other embodiments, n is 1 or 2. In certain other embodiments, p is 2 and n is 1.

In certain embodiments, the compound is a compound of formula I-C1:

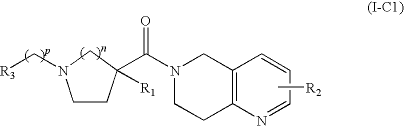

or a pharmaceutically acceptable salt thereof, wherein:

R₁ is alkoxyalkyl;

R₂ is alkyl, haloalkyl, halogen or alkoxy;

R₃ is

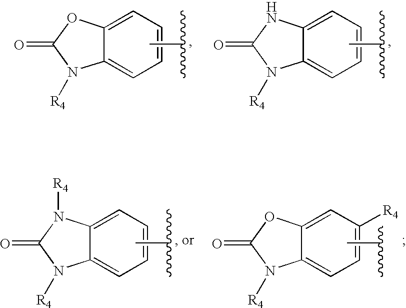

R₄ represents independently for each occurrence hydrogen or alkyl; and

n and p each represent independently 1 or 2.

In certain embodiments, R₁ is —(CH₂)ₓ—O—(CH₂)ₓ—H, wherein X represents independently 1, 2, or 3. In certain embodiments, R₂ is haloalkyl. In certain other embodiments, R₂ is —CF₃. In certain embodiments, wherein R₃ is

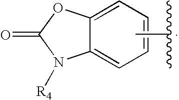

In certain embodiments, R₄ represents independently methyl, ethyl, or propyl. In certain embodiments, n and p are 1.

In certain embodiments, the compound is (1-(4-hydroxy-3-methoxybenzyl)-4-isobutylpiperidin-4-yl)(3-(trifluoromethyl)-7,8-dihydro-1,6-naphthyridin-6(5H)-yl)methanone; 7-((4-isobutyl-4-(3-(trifluoromethyl)-5,6,7,8-tetrahydro-1,6-naphthyridine-6-carbonyl)piperidin-1-yl)methyl)-1-methylquinolin-2(1H)-one; (3-(cyclopropylmethyl)-1-(4-hydroxy-3-methoxybenzyl)pyrrolidin-3-yl)(3-(trifluoromethyl)-7,8-dihydro-1,6-naphthyridin-6(5H)-yl)methanone; 7-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (S)-7-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (R)-7-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 3-(cyclopropylmethyl)-1-(3-fluoro-4-hydroxybenzyl)pyrrolidin-3-yl)(3-(trifluoromethyl)-7,8-dihydro-1,6-naphthyridin-6(5H)-yl)methanone; (3-(cyclopropylmethyl)-1-((tetrahydro-2H-pyran-4-yl)methyl)pyrrolidin-3-yl)(3-(trifluoromethyl)-7,8-dihydro-1,6-naphthyridin-6(5H)-yl)methanone; or a pharmaceutically acceptable salt thereof.

In certain other embodiments, the compound is (3-(cyclopropylmethyl)-1-((2,2-dimethyltetrahydro-2H-pyran-4-yl)methyl)pyrrolidin-3-yl)(3-(trifluoromethyl)-7,8-dihydro-1,6-naphthyridin-6(5H)-yl)methanone; (3-(cyclopropylmethyl)-1-((5-methoxy-2-methyl-2,3-dihydrobenzofuran-6-yl)methyl)pyrrolidin-3-yl)(3-(trifluoromethyl)-7,8-dihydro-1,6-naphthyridin-6(5H)-yl)methanone; 5-((3-(cyclopropylmethyl)-3-(3-(trifluoromethyl)-5,6,7,8-tetrahydro-1,6-naphthyridine-6-carbonyl)pyrrolidin-1-yl)methyl)-3-methylbenzo[d]thiazol-2(3H)-one; 5-((3-(cyclopropylmethyl)-3-(3-(trifluoromethyl)-5,6,7,8-tetrahydro-1,6-naphthyridine-6-carbonyl)pyrrolidin-1-yl)methyl)benzo[d]thiazol-2(3H)-one; 3-(cyclopropylmethyl)-1-(4-hydroxy-4-(6-methoxypyridin-3-yl)cyclohexyl)pyrrolidin-3-yl)(3-(trifluoromethyl)-7,8-dihydro-1,6-naphthyridin-6(5H)-yl)methanone; (3-(cyclopropylmethyl)-1-(2,2-dimethylchromoa-6-yl)methyl)pyrrolidin-3-yl)(3-(trifluoromethyl)-7,8-dihydro-1,6-naphthyridin-6(5H)-yl)methanone; 7-((3-(cyclopropylmethyl)-3-(3-(trifluoromethyl)-5,6,7,8-tetrahydro-1,6-naphthyridin-6-carbonyl)pyrrolidin-1-yl)methyl)quinolin-2(1H)-one; 5-(1-(3-(cyclopropylmethyl)-3-(3-(trifluoromethyl)-5,6,7,8-tetrahydro-1,6-naphthyridine-6-carbonyl)pyrrolidin-1-yl)ethyl)-1,3-dimethyl-1H-benzo[d]imidazol-2(3H)-one; (1-((1H-indazol-5-yl)methyl)-3-(cyclopropylmethyl)pyrrolidin-3-yl)(3-(trifluoromethyl)-7,8-dihydro-1,6-naphthyridin-6(5H)-yl)methanone; (3-(cyclopropylmethyl)-1-(4-hydroxy-4-(6-methoxypyridin-3-yl)cyclohexyl)pyrrolidin-3-yl)(3-(trifluoromethyl)-7,8-dihydro-1,6-naphthyridin-6(5H)-yl)methanone; 6-((3-(cyclobutylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 6-((3-isopentyl-3-(3-(trifluoromethyl)-5,6,7,8-tetrahydro-1,6-naphthyridine-6-carbonyl)pyrrolidin-1-yl)methyl)-1-methylquinolin-2(1H)-one; 6-((3-benzyl-3-(3-(trifluoromethyl)-5,6,7,8-tetrahydro-1,6-naphthyridine-6-carbonyl)pyrrolidin-1-yl)methyl)-1-methylquinolin-2(1H)-one; 6-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 5-((3-(cyclopropylmethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 5-((3-isobutyl-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (S)-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (R)-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 5-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]thiazol-2(3H)-one; 5-(1-(3-(methoxymethyl)-3-(3-(trifluoromethyl)-5,6,7,8-tetrahydro-1,6-naphthyridine-6-carbonyl)pyrrolidin-1-yl)ethyl)-1,3-dimethyl-1H-benzo[d]imidazol-2(3H)-one; (1-(4-hydroxy-4-(6-methoxypyridin-3-yl)cyclohexyl)-3-(methoxymethyl)pyrrolidin-3-yl)(3-(trifluoromethyl)-7,8-dihydro-1,6-naphthyridin-6(5H)-yl)methanone; 6-((3-(methoxymethyl)-3-(3-(trifluoromethyl)-5,6,7,8-tetrahydro-1,6-naphthyridine-6-carbonyl)pyrrolidin-1-yl)methyl)-4-methyl-2H-benzo[b][1,4]oxazin-3(4H)-one; 6-(1-(3-(methoxymethyl)-3-(3-(trifluoromethyl)-5,6,7,8-tetrahydro-1,6-naphthyridine-6-carbonyl)pyrrolidin-1-yl)ethyl)-4-methyl-2H-benzo[b][1,4]oxazin-3(4H)-one; 5-(1-(3-(methoxymethyl)-3-(3-(trifluoromethyl)-5,6,7,8-tetrahydro-1,6-naphthyridine-6-carbonyl)pyrrolidin-1-yl)ethyl)-3-methylbenzo[d]oxazol-2(3H)-one; (1-(4-cyclopropyl-4-hydroxycyclohexyl)-3-(methoxymethyl)pyrrolidin-3-yl)(3-trifluoromethyl)-7,8-dihydro-1,6-naphthyridin-6(5H)-yl)methanone; 6-chloro-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 6-chloro-5-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 5-((3-(methoxymethyl)-3-(3-(trifluoromethyl)-5,6,7,8-tetrahydro-1,6-naphthyridine-6-carbonyl)pyrrolidin-1-yl)methyl)-1,3-dimethyl-1H-benzo[d]imidazol-2(3H)-one; 5-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; (R)-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one and (S)-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 3-ethyl-6-((3-(methoxymethyl)-3-(3-(trifluoromethyl)-5,6,7,8-tetrahydro-1,6-naphthyridine-6-carbonyl)pyrrolidin-1-yl)methyl)benzo[d]oxazol-2(3H)-one; 5-bromo-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 6-((3-(methoxymethyl)-3-(3-(trifluoromethyl)-5,6,7,8-tetrahydro-1,6-naphthyridine-6-carbonyl)pyrrolidin-1-yl)methyl)-3,5-dimethylbenzo[d]oxazol-2(3H)-one; 5-((3-(ethoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 5-((3-(methoxymethyl)-3-(7-(trifluoromethyl)-1,2,3,4-tetrahydroisoquinoline-2-carbonyl)pyrrolidin-1-yl)methyl)-3-methylbenzo[d]oxazol-2(3H)-one; 5-((3-isopropyl-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 2-(1-((3-methyl-2-oxo-2,3-dihydrobenzo[d]oxazol-5-yl)methyl)-3-(3-(trifluoromethyl)-5,6,7,8-tetrahydro-1,6-naphthyridine-6-carbonyl)pyrrolidin-3-yl)acetonitrile; 5-((3-(hydroxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; (3-(methoxymethyl)-1-(4-(6-methoxypyridin-3-yl)cyclohexyl)pyrrolidin-3-yl)(3-(trifluoromethyl)-7,8-dihydro-1,6-naphthyridin-6(5H)-yl)methanone; (1-(4-hydroxy-4-(pyrimidin-5-yl)cyclohexyl)-3-(methoxymethyl)pyrrolidin-3-yl)(3-(trifluoromethyl)-7,8-dihydro-1,6-naphthyridin-6(5H)-yl)methanone; (3-(methoxymethyl)-1-(4-(pyrimidin-5-yl)cyclohexyl)pyrrolidin-3-yl)(3-(trifluoromethyl)-7,8-dihydro-1,6-naphthyridin-6(5H)-yl)methanone; (1-(4-(4-fluorophenyl)cyclohexyl)-3-(methoxymethyl)pyrrolidin-3-yl)(3-(trifluoromethyl)-7,8-dihydro-1,6-naphthyridin-6(5H)-yl)methanone; 6-((4-isobutyl-4-(3-(trifluoromethyl)-5,6,7,8-tetrahydro-1,6-naphthyridine-6-carbonyl)piperidin-1-yl)methyl)-3-methylbenzo[d]thiazol-2(3H)-one; 6-((4-isobutyl-4-(3-(trifluoromethyl)-5,6,7,8-tetrahydro-1,6-naphthyridine-6-carbonyl)piperidin-1-yl)methyl)-3-methylbenzo[d]oxazol-2(3H)-one; 2-(3-(methoxymethyl)-1-(3-methyl-2-oxo-2,3-dihydrobenzo[d]oxazol-5-yl)methyl)pyrrolidine-3-carbonyl)-7-(trifluoromethyl)-1,2,3,4-tetrahydroisoquinoline-5-carbonitrile; 5-((3-(methoxymethyl)-3-(7-(trifluoromethyl)-1,2,3,4-tetrahydroisoquinoline-2-carbonyl)pyrrolidin-1-yl)methyl)-3-methylbenzo[d]oxazol-2(3H)-one; 6-(3-(methoxymethyl)-1-((3-methyl-2-oxo-2,3-dihydrobenzo[d]oxazol-5-yl)methyl)pyrrolidine-3-carbonyl)-5,6,7,8-tetrahydro-1,6-naphthyridine-3-carbonitrile; 5-((3-(methoxymethyl)-3-(1,2,3,4-tetrahydroisoquinoline-2-carbonyl)pyrrolidin-1-yl)methyl)-3-methylbenzo[d]oxazol-2(3H)-one; 5-((3-(methoxymethyl)-3-(3-(trifluoromethyl)-6,7-dihydro-5H-pyrrolo[3,4-b]pyridine-6-carbonyl)pyrrolidin-1-yl)methyl)-3-methylbenzo[d]oxazol-2(3H)-one; 5-(2-(3-(methoxymethyl)-3-(3-(trifluoromethyl)-5,6,7,8-tetrahydro-1,6-naphthyridine-6-carbonyl)pyrrolidin-1-yl)propan-2-yl)-3-methylbenzo[d]oxazol-2(3H)-one; 5-(1-(3-(methoxymethyl)-3-(3-(trifluoromethyl)-5,6,7,8-tetrahydro-1,6-naphthyridine-6-carbonyl)pyrrolidin-1-yl)cyclopropyl)-3-methylbenzo[d]oxazol-2(3H)-one; 6-(1-(3-(ethoxymethyl)-3-(3-(trifluoromethyl)-5,6,7,8-tetrahydro-1,6-naphthyridine-6-carbonyl)pyrrolidin-1-yl)ethyl)-4-methyl-2H-benzo[b][1,4]oxazin-3(4H)-one; 7-((3-(methoxymethyl)-3-(3-(trifluoromethyl)-5,6,7,8-tetrahydro-1,6-naphthyridine-6-carbonyl)pyrrolidin-1-yl)methyl)-1-methylquinoxalin-2(1H)-one; 7-(1-(3-(methoxymethyl)-3-(3-(trifluoromethyl)-5,6,7,8-tetrahydro-1,6-naphthyridine-6-carbonyl)pyrrolidin-1-yl)ethyl)-1-methylquinoxalin-2(1H)-one; 5-(3-(methoxymethyl)-3-(3-(trifluoromethyl)-5,6,7,8-tetrahydro-1,6-naphthyridine-6-carbonyl)pyrrolidin-1-yl)-3-methyl-3,5,6,7-tetrahydro-2H-indeno[5,6-d]oxazol-2-one; 6-(3-(methoxymethyl)-3-(3-(trifluoromethyl)-5,6,7,8-tetrahydro-1,6-naphthyridine-6-carbonyl)pyrrolidin-1-yl)-1-methyl-7,8-dihydro-1H-indeno[4,5-d]oxazol-2(6H)-one; 6-((3-(methoxymethyl)-3-(3-(trifluoromethyl)-5,6,7,8-tetrahydro-1,6-naphthyridine-6-carbonyl)pyrrolidin-1-yl)methyl)-1-methylindolin-2-one; 6-(1-(3-(methoxymethyl)-3-(3-(trifluoromethyl)-5,6,7,8-tetrahydro-1,6-naphthyridine-6-carbonyl)pyrrolidin-1-yl)ethyl)-1-methylindolin-2-one; (1-(4-(5-fluoropyridin-2-yl)cyclohexyl)-3-(methoxymethyl)pyrrolidin-3-yl)(3-(trifluoromethyl)-7,8-dihydro-1,6-naphthyridin-6(5H)-yl)methanone; (1-(4-fluoro-4-(6-methoxypyridin-3-yl)cyclohexyl)-3-(methoxymethyl)pyrrolidin-3-yl)(3-(trifluoromethyl)-7,8-dihydro-1,6-naphthyridin-6(5H)-yl)methanone; (3-(methoxymethyl)-1-(4-(pyrimidin-2-yl)cyclohexyl)pyrrolidin-3-yl)(3-(trifluoromethyl)-7,8-dihydro-1,6-naphthyridin-6(5H)-yl)methanone; or a pharmaceutically acceptable salt thereof

In certain embodiments, the compound is:

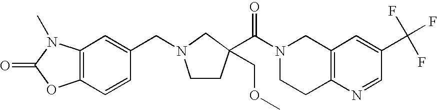

or a pharmaceutically acceptable salt thereof. In certain other embodiments, the compound is:

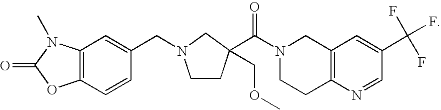

In certain embodiments, the disorder is wet age-related macular degeneration, choroidal neovascularization, diabetic retinopathy, intraocular neovascularization, retinal vein occlusion, retinopathy of prematurity, angioid streak, high myopia, macular edema, ocular histoplasmosis, retinal detachment, or retinitis pigmentosa. In certain other embodiments, the disorder is wet age-related macular degeneration. In certain other embodiments, the disorder is choroidal neovascularization or diabetic retinopathy. In certain embodiments, the patient is a human.

The invention further provides use of a compound described herein, such as a compound of Formula I, in the manufacture of a medicament for the treatment of an opthalmological disorder described herein.

The capacity of the compounds described herein to antagonize CCR2 function can be determined using a suitable screen (e.g., high throughput assay). For example, an agent can be tested in an extracellular acidification assay, calcium flux assay, ligand binding assay or chemotaxis assay (see, for example, Hesselgesser et al., J Biol. Chem. 273(25):15687-15692 (1998), WO 00/05265 and WO 98/02151).

“Prodrug” includes compounds that are transformed in vivo to yield a compound of Formula (I) or a pharmaceutically acceptable salt, hydrate or solvate of the compound. The transformation may occur by various mechanisms, such as through hydrolysis in blood. For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of Formula (I) contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(_{C1}-C₆)alkyl)₂ or glycosyl (the radical resulting from the removal of a hydroxyl group of the hemiacetal form of a carbohydrate).

If a compound of Formula (I) incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino, morpholino, piperidin-1-yl or pyrrolidin-1-yl.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the invention. In addition, the invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column.

The compounds of Formula (I) may exist in unsolvated as well as solvated forms with pharmaceutically acceptable solvents such as water, ethanol, and the like, and it is intended that the invention embrace both solvated and unsolvated forms.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of Formula (I) can generally be prepared by following procedures analogous to those disclosed in the Schemes and/or in the Examples hereinbelow, by substituting an isotopically labeled reagent for a non-isotopically labeled reagent.

The compounds of the invention may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

The compounds of the invention can be administered to a patient at dosage levels in the range of from about 0.01 to about 100 mg per day. As used herein, the term “unit dose” or “unit dosage” refers to physically discrete units that contain a predetermined quantity of a compound of the invention calculated to produce a desired therapeutic effect. The dosage to be administered may vary depending upon the physical characteristics of the patient, the severity of the patient's symptoms, and the means used to administer the drug. The specific dose for a given patient is usually set by the judgment of the attending physician. It is also noted that the compounds of the invention can be used in sustained release, controlled release, and delayed release formulations, which forms are also well known to one of ordinary skill in the art.

The compositions and combination therapies of the invention may be administered in combination with a variety of pharmaceutical excipients, including stabilizing agents, carriers and/or encapsulation formulations as described herein.

Aqueous compositions of the present invention comprise an effective amount of the peptides of the invention, dissolved or dispersed in a pharmaceutically acceptable carrier or aqueous medium.

“Pharmaceutically or pharmacologically acceptable” include molecular entities and compositions that do not produce an adverse, allergic or other untoward reaction when administered to an animal, or a human, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For human administration, preparations should meet sterility, pyrogenicity, general safety and purity standards as required by FDA Office of Biologics standards.

The pharmaceutical compositions of this invention may be used in the form of a pharmaceutical preparation, for example, in solid, semisolid or liquid form, which contains one or more of the compound of the invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the invention may be incorporated for administration orally or by injection include aqueous solution, suitably flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

For treating clinical conditions and diseases noted above, the compound of this invention may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

It is contemplated that the compounds described herein may be administered to a patient using standard drug delivery methods known in the art, such as (1) oral administration in the form of, e.g., drenches (aqueous or non-aqueous solutions or suspensions) and tablets, e.g., those targeted for buccal, sublingual, and systemic absorption, boluses, powders, granules, and pastes for application to the tongue; (2) parenteral administration, such as by subcutaneous, intramuscular, intravenous or epidural injection; (3) ocularly, such as by intravitreal injection; (4) topical administration, such as in the form of a cream, ointment, or a controlled-release patch or spray applied to the skin; (5) intravaginally or intrarectally, for example, as a pessary, cream or foam; (6) sublingually; (7) transdermally; or (8) nasally. Administration of the compounds using an intraocular drug delivery device or implant is also contemplated. Various intraocular drug delivery devices and implants are known in the art and are contemplated to be amenable to administration of the compounds described herein. See for example, U.S. Pat. Nos. 6,074,661; 6,331,313; 6,369,116; and 6,699,493; and US Patent Application Publication Nos. 20070059336, 20060182783, and 20060110429, each of which are hereby incorporated by reference. In certain embodiments, the intraocular drug delivery device or implant provides for controlled/slow release of a compound described herein.

The preparation of an aqueous composition that contains a composition of the invention or an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Pharmaceutically acceptable salts include acid addition salts and which are formed with inorganic acids such as, for example, hydrochloric, hydrobromic, boric, phosphoric, sulfuric acids or phosphoric acids, or such organic acids as acetic, oxalic, tartaric, maleic, fumaric, citric, succinic, mesylic, mandelic, succinic, benzoic, ascorbic, methanesulphonic, α-keto glutaric, α-glycerophosphoric, glucose-1-phosphoric acids and the like. Salts formed with the free carboxyl groups can also be derived from inorganic bases such as, for example, sodium, potassium, ammonium, calcium, magnesium, or ferric hydroxides, and such organic bases as isopropylamine, trimethylamine, histidine, procaine and the like. Other examples of pharmaceutically acceptable salts include quaternary derivatives of the compounds of Formula I, such as the compounds quaternized by compounds Rₓ-T wherein Rₓ is C₁₋₆ alkyl, phenyl-C₁₋₆ alkyl or C₅₋₇ cycloalkyl, and T is a radical corresponding to an anion of an acid. Suitable examples of Rₓ include methyl, ethyl and n- and iso-propyl; and benzyl and phenethyl. Suitable examples of T include halide, e.g., chloride, bromide or iodide. Yet other examples of pharmaceutically acceptable salts also include internal salts such as N-oxides. It is contemplated that the compounds described herein may exist in a salt form.

Therapeutic or pharmacological compositions of the present invention will generally comprise an effective amount of the component(s) of the combination therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including cremes, lotions, mouthwashes, inhalants and the like.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze drying techniques which yield a powder of the active ingredient plus any additional desired ingredient from a previously sterile-filtered solution thereof.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration; liposomal formulations; time-release capsules; and any other form currently used, including creams.

The use of sterile formulations, such as saline-based washes, by surgeons, physicians or health care workers to cleanse a particular area in the operating field may also be particularly useful. Therapeutic formulations in accordance with the present invention may also be reconstituted in the form of mouthwashes, or in conjunction with antifungal reagents Inhalant forms are also envisioned. The therapeutic formulations of the invention may also be prepared in forms suitable for topical administration, such as in cremes and lotions.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals. For example, for parenteral administration, a suitably buffered, and if necessary, isotonic aqueous solution would be prepared and used for intravenous, intramuscular, subcutaneous or even intraperitoneal administration. One dosage could be dissolved in 1 mL of isotonic NaCl solution and either added to 1000 mL of hypodermolysis fluid or injected at the proposed site of infusion, (see for example, Remington's Pharmaceutical Sciences 15th Edition, pages 1035-1038 and 1570-1580).

In certain embodiments, active compounds may be administered orally. This is contemplated for agents which are generally resistant, or have been rendered resistant, to proteolysis by digestive enzymes. Such compounds are contemplated to include chemically designed or modified agents; dextrorotatory peptides; and peptide and liposomal formulations in time release capsules to avoid peptidase and lipase degradation.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Advantageously, the invention also provides kits for use by a consumer having, or at risk of having, a disease or condition described herein, which can be ameliorated by a CCR2 antagonist. Such kits include a suitable dosage form such as those described above and instructions describing the method of using such dosage form to mediate, reduce or prevent an opthalmological disorder described herein. The instructions would direct the consumer or medical personnel to administer the dosage form according to administration modes known to those skilled in the art. Such kits could advantageously be packaged and sold in single or multiple kit units.

Since the invention has an aspect that relates to the treatment of the disease/conditions described herein with a combination of active ingredients which may be administered separately, the invention also relates to combining separate pharmaceutical compositions in kit form. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### Biological Activity of Compounds

The suitability of compounds described herein for the uses described herein may be determined by methods and assays known in the art. The following tests are found particularly advantageous.

For determining the ability of compounds to effect chemotaxis, assays in two formats may be used:

Methods using Boyden chambers: Cells are washed twice in RPMI with 0.1% BSA and starved for 2 hours in RPMI 0.1% BSA at 37° C. in 5% CO₂. After starving, the cells are resuspended at 1×10⁶ cell/mL (in some cases, the cell density may be varied in order to investigate the optimal cell numbers that can be used in the assay) in RPMI 0.1% BSA. About 1×10⁵/100 μL cells are added into the upper wells of the Boyden chamber apparatus with 8 μm pore size filter. Chemotactic factors are diluted to the indicated concentrations in RPMI 0.1% BSA, and 200 μL of the mixture is added into the lower wells of the Boyden chambers. After 2 hours at 37° C. in 5% CO₂, the cells remaining in the upper chamber are removed. Migrated cells in the lower surface of the filters are fixed with Methanol and stained with 15% Giemsa. The cells are counted in 10 high power fields.

Methods using neuroprobes: Cells are washed twice in RPMI with 0.1% BSA and starved for 2 hours in RPMI 0.1% BSA at 37° C. in 5% CO₂. After starving, the cells are resuspended at 1×10⁶ cell/mL in RPMI 0.1% BSA and stained with 1 μg/mL Calcein AM for 30 min at 37° C. in 5% CO₂. Stained cells are washed twice with PBS and resuspended at 1×10⁶ cell/mL in RPMI 0.1% BSA. About 25 μL of the cells are added into the upper chambers of the 96-well neuroprobe plates with an 8 μm pore size filter. Chemotactic factors are diluted to the indicated concentrations in RPMI 0.1% BSA, and 30 μL of the mixture is added into the lower chambers of the 96-well neuroprobe plate. After 2 hours at 37° C. in 5% CO₂, the cells remaining in the upper chambers are removed and rinsed with PBS once. Migrated cells in the lower surface of the filters and low chamber are determined as the fluorescent value measured at λ450-530 by Cytofluor.

For determining the ability of compounds to bind to CCR2 and to block MCP-1 binding, the following assay is useful. To maximize reliability and reproducibility Human recombinant CHO-K1 cells that overexpress CCR2 are used in this assay. Increasing concentrations of antagonist is incubated with cells in the presence of 1% DMSO, 25 mM HEPES pH:7.4, 1 mM CaCl₂, 0.5% BSA, 5 mM MgCl₂, 0.1% sodium azide. The potency of the compounds is calculated as a function of decreasing quantity of ¹²⁵I-labeled MCP-1 (1 nM) ability to bind to the receptor. Reference standards are run as an integral part of each assay to ensure the validity of the results obtained. Where presented, IC₅₀ values are determined by a non-linear, least squares regression analysis using Data Analysis Toolbox (MDL Information Systems, San Leandro, Calif., USA). Where inhibition constants Kᵢ are presented, the Kᵢ values are calculated using the equation of Cheng and Prusoff (Cheng, Y., Prusoff, W.H., Biochem. Pharmacol. 22:3099-3108, 1973) using the observed IC₅₀ of the tested compound, the concentration of radioligand employed in the assay, and the historical values for the K_{D} of the ligand (obtained experimentally at MDS Pharma Services). Where presented, the Hill coefficient (n_{H}), defining the slope of the competitive binding curve, is calculated using Data Analysis Toolbox. Hill coefficients significantly different than 1.0 may suggest that the binding displacement does not follow the laws of mass action with a single binding site. Where IC₅₀, and/or n_{H} data are presented without Standard Error of the Mean (SEM), data are insufficient to be quantitative, and the values presented (Kᵢ, IC₅₀, n_{H}) should be interpreted accordingly.

The efficacy of compounds of the invention may further be determined using a (GTP γ S) assay in which the potency of a given antagonist is assessed by the inhibition observed in the binding of radioactively labeled GTP to the cell membranes or whole cells. Compounds are tested at several concentrations in duplicate (n=2) to obtain a dose-response curve and estimated IC₅₀ values. The assay buffer is 20 mM HEPES pH 7.4; 100 mM NaCl, 10 μg/mL saponin, 1 mM MgCl₂. The assay is performed on membranes that are thawed on ice and diluted in assay buffer to give 250 μg/mL (5 μg/20 μL), keep on ice. 20 μL of 5 μM GDP (1 μM final). 10 μL of antagonist at increasing concentrations is added successively in the wells of an Optiplate (Perkin Elmer) together with 20 μL of membranes (5 μg) and preincubated for 15 min. at room temperature. To this, 10 μL of assay buffer or of reference agonist (MCP-1 R&D Systems, 279-MC) at EC₈₀ (10×), 20 μL of GTPg³⁵S (0.1 nM final), 20 μL of PVT-WGA beads (Amersham, RPNQ001). Control antagonist RS 102895 (Tocris, 2089) diluted in assay buffer is used in each assay as a reference. The plate is covered with a topseal, placed on an orbital shaker for 2 min., incubated for 30 min. at room temperature, centrifuged for 10 min. at 2000 rpm, incubated for 2 h at room temperature and counted in a TopCount (Packard) for 1 min.

Efficacy of the compounds described herein for treating wet age-related macular degeneration, choroidal neovascularization, diabetic retinopathy, intraocular neovascularization, retinal vein occlusion, retinopathy of prematurity, angioid streak, high myopia, macular edema, and ocular histoplasmosis can be evaluated using procedures known in the art.

### Preparation of Compounds

Compounds of the invention may be prepared as described in the following schemes. Starting materials are either commercially available or made by known procedures in the reported literature or as illustrated. This invention provides procedures for the preparation of compounds of formula I as defined above, which comprises different sequences of assembling intermediates of formula (II), formula (III), formula (IV), formula (VII) and formula (VIII).

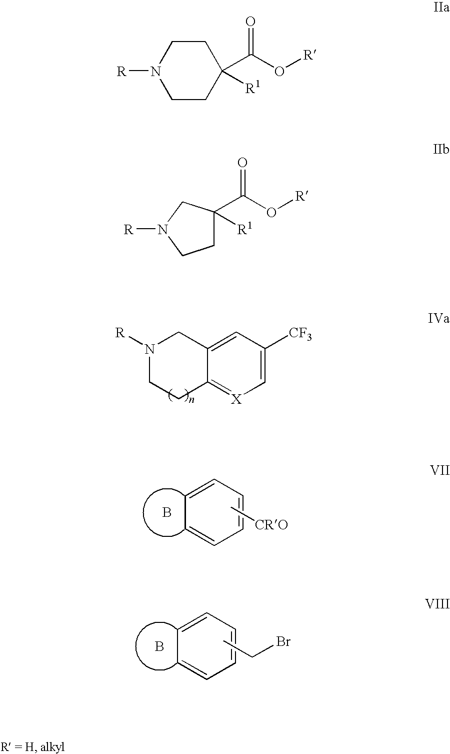

wherein B, and R¹ are defined as in formula I, X and Y can be C or NR′, R′ is H or alkyl, R represents either hydrogen or a protecting group.

General procedures for preparing target molecules Ia and Ib by using intermediates II, III, IV, V, VI, VII and VIII are outlined in Scheme 1 and Scheme 2. Coupling of acid (III) and amine (IV) under standard amide bond formation using activating reagent such as HOBt/EDCI, PyBrop/EDCI with or without catalyst DMAP gives the intermediates Va and Vb. Removal of the Boc protecting group yields the amines (VI). Reductive amination with aldehydes or ketones (VII) in the presence of a reducing reagent such as sodium triacetoxyborohydride and sodium cyanoborohydride finally provides compound I. Alkylation of amine (VI) with an alkyl halide or aryl halide (VIII) in the presence of base such as K₂CO₃, or Cs₂CO₃ combined with organic base such as DIEA or TEA under elevated temperature also yield the compound of formula I. A mixture of enantiomers, diastereomers, cis/trans isomers resulted from the process can be separated into their single components by chiral salt technique, chromatography using normal phase, reverse phase or chiral column, depending on the nature of the separation.

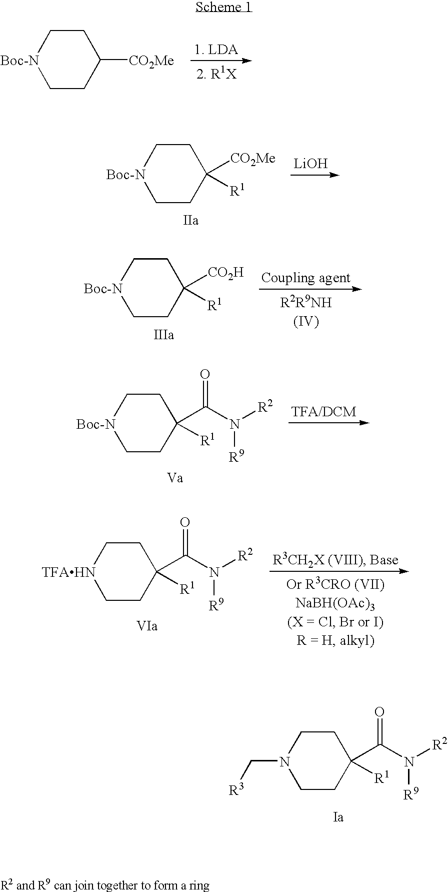

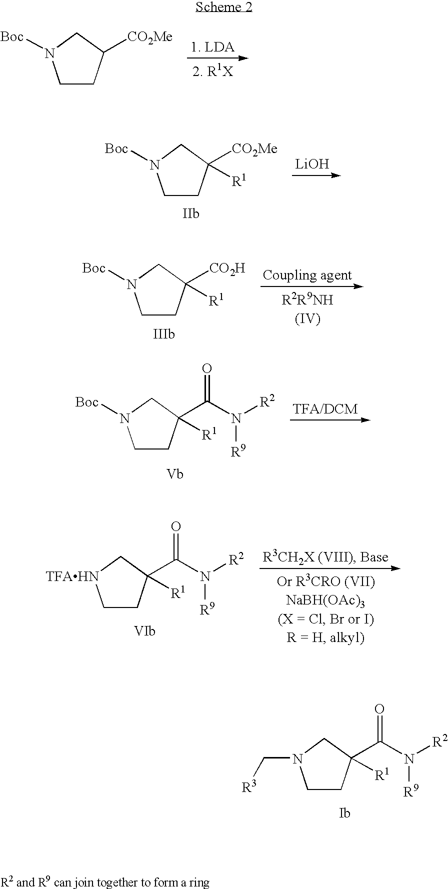

The procedures for preparation of intermediate VIIIa, VIIIb are illustrated in Scheme 3. Intermediate (VIIa), 7-(bromomethyl)-1-methylquinolin-2(1H)-one is prepared by following the literature procedure (Bioorg. Med. Chem. Lett., 17(4), 874-878; 2007).

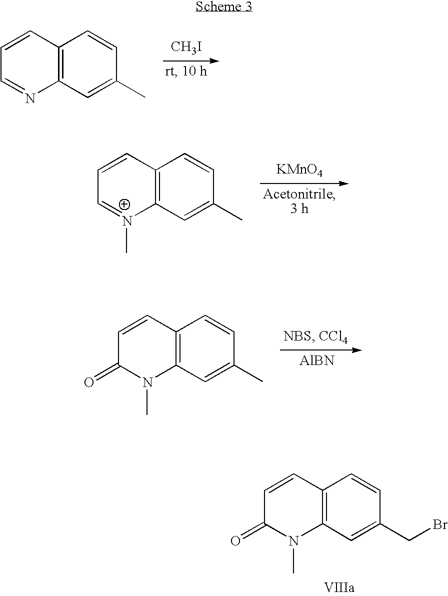

Synthesis of 3-(trifluoromethyl)-5,6,7,8-tetrahydro-1,6-naphthyridine (IVa) has been reported in literature (WO 2005/105092). The preparative procedures for target molecule Example 1 and 3 are illustrated in Scheme 4 and 5. The coupling of acid (III) and amine (IV) under standard amide bond formation using activating reagent such as HOBt/EDCI was unsuccessful to give desired intermediates Va and Vb. An alternative method is the conversion of acid (III) to acid chloride (IX) using thionyl chloride, followed by coupling reaction with amine (IV) in presence of triethylamine. Removal of the Boc protecting group with 20% TFA in DCM gave the amines (VI). Alkylation of amine (VI) with the intermediate (VIII) in the presence of K₂CO₃ and DIEA was carried out in a Microwave instrument at 110° C. to yield Example 1 and 3.

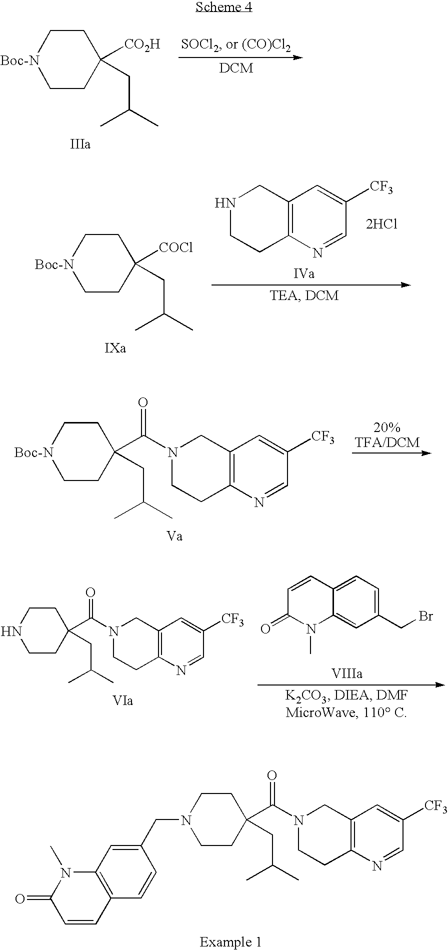

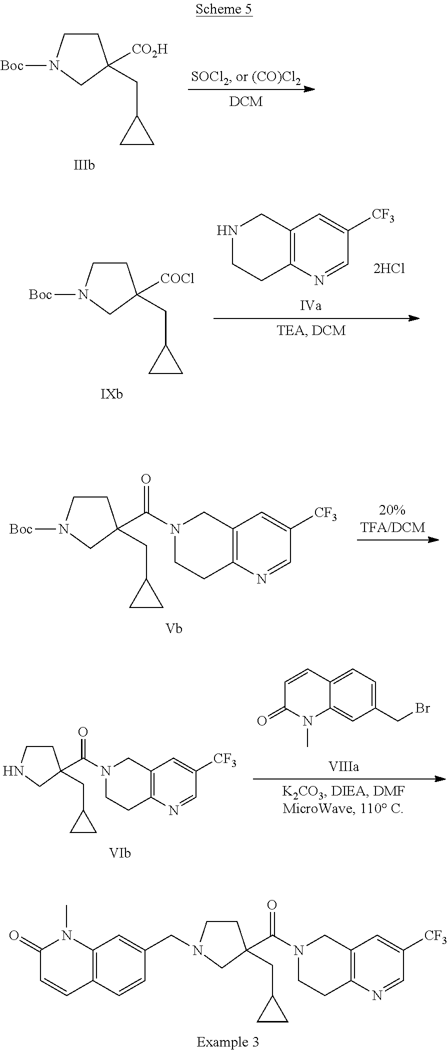

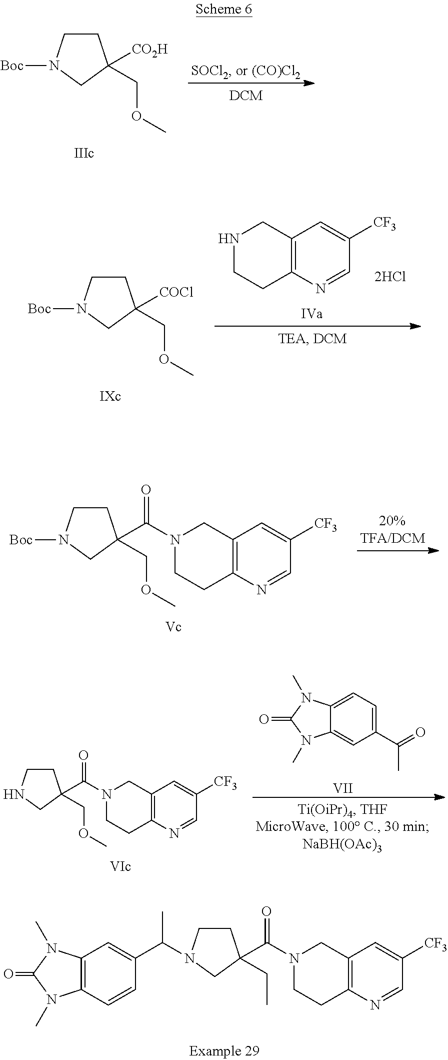

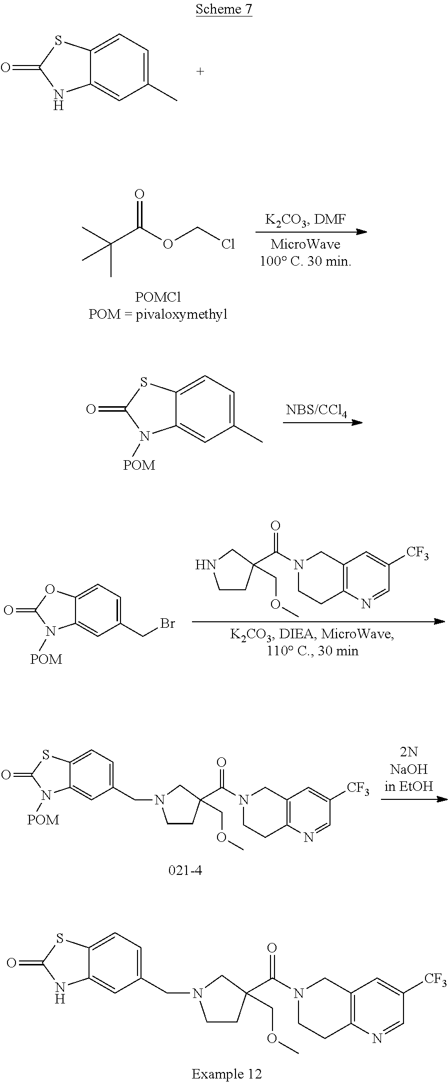

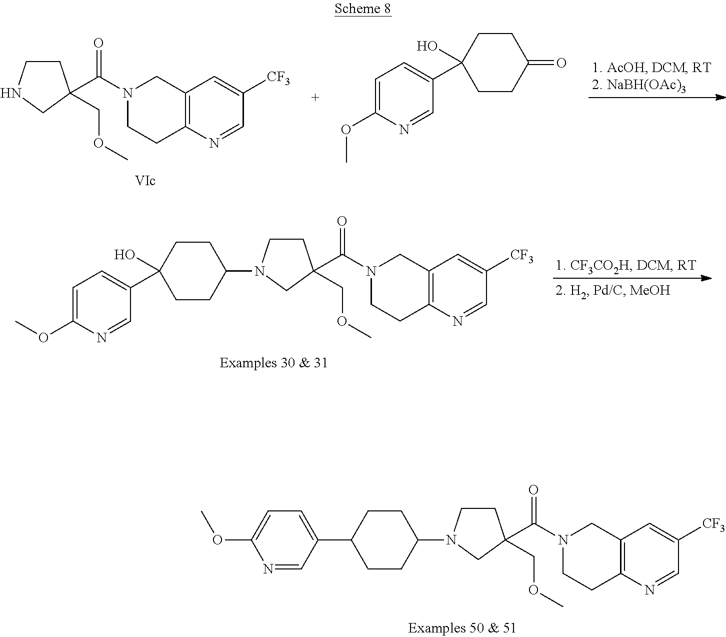

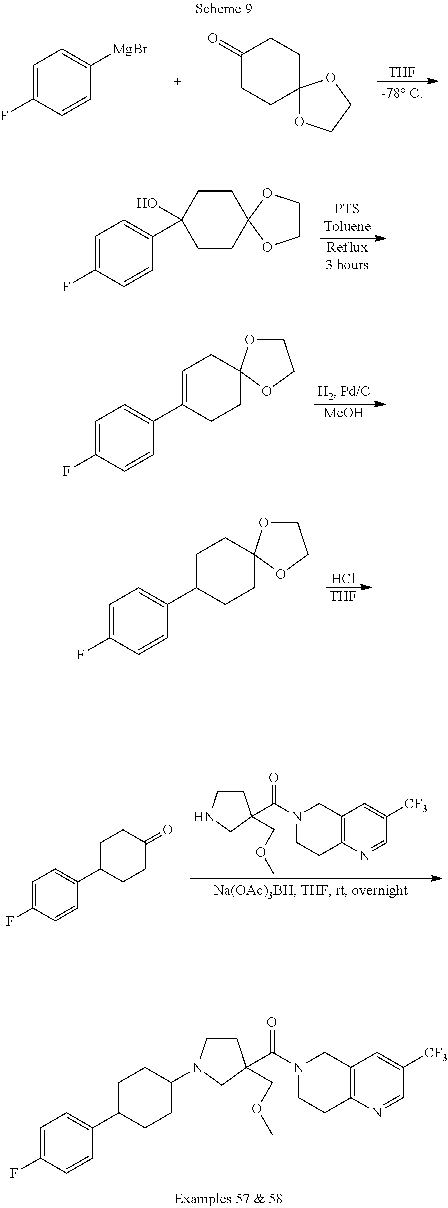

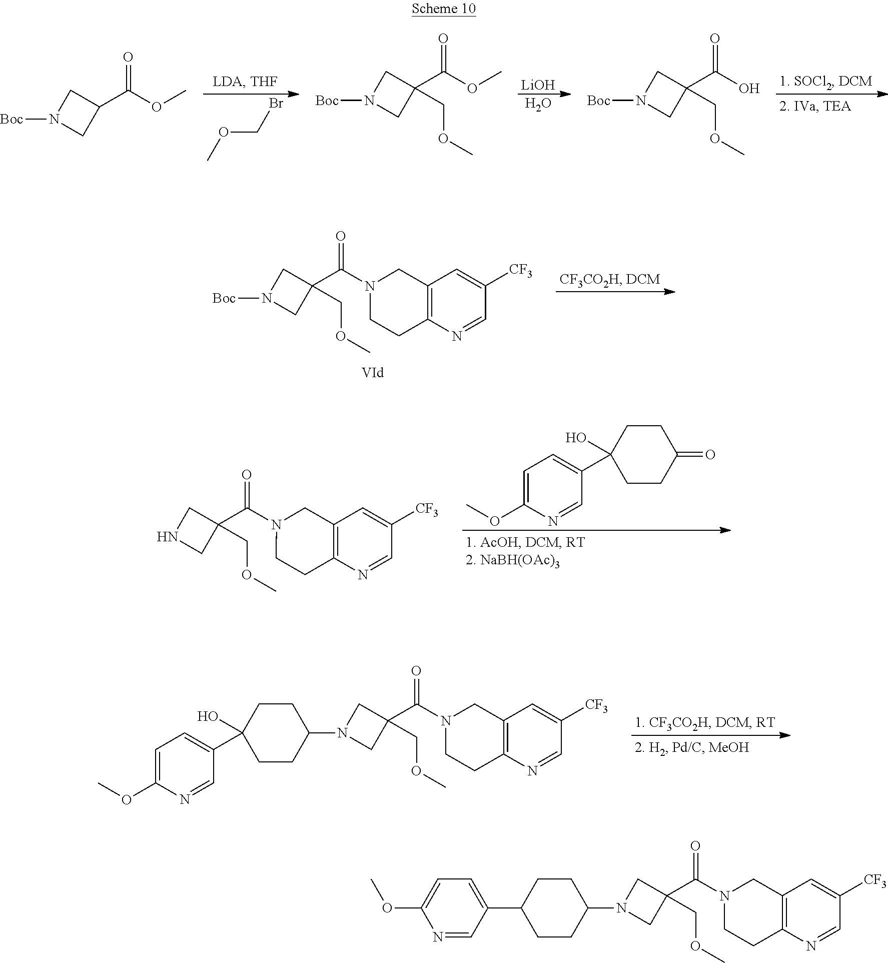

### DEFINITIONS

For convenience, certain terms used throughout the specification, examples, and appended claims are collected here.

“CCR2 receptor modulator” or “CCR2 modulator” includes compounds having effect at the CCR2 receptors, including those compounds having a modulating effect primarily at CCR2.

“Treating”, includes any effect, e.g., lessening, reducing, modulating, or eliminating, that results in the improvement of the condition, disease, disorder and the like.

The symbol “ [symbol] ” indicates a point of attachment.

“Alkyl” includes saturated aliphatic groups, e.g., straight-chain alkyl groups such as methyl, ethyl, propyl, butyl, pentyl, hexyl, heptyl, octyl, nonyl, and decyl; branched-chain alkyl groups (e.g., isopropyl, tert-butyl, and isobutyl); cycloalkyl (alicyclic) groups like cyclopropyl, cyclopentyl, cyclohexyl, cycloheptyl, and cyclooctyl); lower alkyl-substituted cycloalkyl groups; and cycloalkyl-substituted alkyl groups. In an embodiment, alicyclic rings do not include bridged rings. In an embodiment, the alkyl group is substituted.

“Alkyl” groups may also optionally include heteroatoms, i.e., where oxygen, nitrogen, sulfur or phosphorous atoms replaces one or more hydrocarbon backbone carbon atoms, particularly where the substitution does not adversely impact the efficacy of the resulting compound.

Straight or branched alkyl groups may have six or fewer carbon atoms in their backbone (e.g., C₁-C₆ for straight chain, C₃-C₆ for branched chain), and more preferably four or fewer. Preferred cycloalkyl groups have from three to eight carbon atoms in their ring structure, and more preferably five or six carbons in the ring structure. “C₁-C₆” includes alkyl groups containing one to six carbon atoms.

“Substituted alkyls” refers to alkyl moieties having substituents replacing a hydrogen on one or more carbons of the hydrocarbon backbone. Such substituents can include alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acylamino, amidino, imino, sulfhydryl, alkylthio, thiocarboxylate, sulfates, alkylsulfinyl, sulfonato, sulfamoyl, sulfonamido, nitro, trifluoromethyl, cyano, azido, or heterocyclyl.

“Alkylene”, unless indicated otherwise, means a straight or branched, saturated aliphatic, divalent radical having the number of carbon atoms indicated (e.g., (C₁₋₆)alkylene includes methylene (—CH₂—), ethylene (—CH₂CH₂—), trimethylene (—CH₂CH₂CH₂—), and the like. Alkylene may be optionally substituted as provided for alkyl, or as otherwise indicated.

“Alkenylene”, unless indicated otherwise, means a straight or branched divalent radical containing a double bond and having the number of carbon atoms indicated; for example, ethylene and propylene. Alkylene may be optionally substituted as provided for alkyl, or as otherwise indicated.

“Alkynylene”, unless indicated otherwise, means a straight or branched, unsaturated divalent radical containing a triple bond and having the number of carbon atoms indicated; for example, ethynylene and propynylene. Alkynylene may be optionally substituted as provided for alkyl, or as otherwise indicated.

“Aryl” includes groups with aromaticity, including 5- and 6-membered unconjugated (i.e., single-ring) aromatic groups that may include from zero to four heteroatoms, as well as conjugated (i.e., multicyclic) systems having at least one ring that is aromatic. Examples of aryl groups include benzene, phenyl, tolyl and the like. Multicyclic aryl groups include tricyclic and bicyclic systems, e.g., naphthalene, benzoxazole, benzodioxazole, benzothiazole, benzoimidazole, benzothiophene, methylenedioxyphenyl, quinoline, isoquinoline, naphthridine, indole, benzofuran, purine, benzofuran, deazapurine, indolizine, tetralin, and methylenedioxyphenyl.

Aryl groups having heteroatoms in the ring structure may also be referred to as “aryl heterocycles”, “heterocycles,” “heteroaryls” or “heteroaromatics”; e.g., pyrrole, furan, thiophene, thiazole, isothiazole, imidazole, triazole, tetrazole, pyrazole, oxazole, isoxazole, pyridine, pyrazine, pyridazine, and pyrimidine. The aromatic ring can be substituted at one or more ring positions with, for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, acylamino, amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Arylene”, unless indicated otherwise, means an aromatic, divalent radical. The aromatic group includes 5- and 6-membered unconjugated (i.e., single-ring) aromatic moieties that may include from zero to four heteroatoms, as well as conjugated (i.e., multicyclic) systems having at least one ring that is aromatic. Arylene may be optionally substituted as described for aryl, or as otherwise indicated.

“Heteroarylene”, unless indicated otherwise, means an heteroaromatic, divalent radical. Heteroarylene may be optionally substituted as described for aryl, or as otherwise indicated.

An “alkylaryl” or an “aralkyl” moiety is an alkyl substituted with an aryl group (e.g., phenylmethyl (benzyl)).

An “alkenylaryl” or an “aralkenyl” moiety is an alkenyl group substituted with an aryl group.

An “alkylheteroaryl” or an “heteroaralkyl” moiety is an alkyl substituted with a heteroaryl group (e.g., phenylmethyl (benzyl)).

An “alkenylheteroaryl” or an “heteroaralkenyl” moiety is an alkenyl group substituted with a heteroaryl group.

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, and decenyl), branched-chain alkenyl groups, cycloalkenyl groups such as cyclopropenyl, cyclopentenyl, cyclohexenyl, cycloheptenyl, and cyclooctenyl; alkyl or alkenyl-substituted cycloalkenyl groups, and cycloalkyl or cycloalkenyl-substituted alkenyl groups.

“Alkenyl” groups may also optionally include heteroatoms, i.e., where oxygen, nitrogen, sulfur or phosphorous atoms replaces one or more hydrocarbon backbone carbon atoms, particularly where the substitution does not adversely impact the efficacy of the resulting compound.

Straight or branched alkenyl groups may have six or fewer carbon atoms in their backbone (e.g., C₂-C₆ for straight chain, C₃-C₆ for branched chain.) Preferred cycloalkenyl groups have from three to eight carbon atoms in their ring structure, and more preferably have five or six carbons in the ring structure. The term “C₂-C₆” includes alkenyl groups containing two to six carbon atoms.

“Substituted alkenyls” refers to alkenyl moieties having substituents replacing a hydrogen on one or more hydrocarbon backbone carbon atoms. Such substituents can include alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acylamino, amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, or heterocyclyl.

“Alkynyl” includes unsaturated aliphatic groups analogous in length and possible substitution to the alkyls described above, but which contain at least one triple bond. For example, “alkynyl” includes straight-chain alkynyl groups (e.g., ethynyl, propynyl, butynyl, pentynyl, hexynyl, heptynyl, octynyl, nonynyl, decynyl), branched-chain alkynyl groups, and cycloalkyl or cycloalkenyl substituted alkynyl groups.

“Alkynyl” groups may also optionally include heteroatoms, i.e., where oxygen, nitrogen, sulfur or phosphorous atoms replaces one or more hydrocarbon backbone carbon atoms, particularly where the substitution does not adversely impact the efficacy of the resulting compound

Straight or branched chain alkynyls group may have six or fewer carbon atoms in their backbone (e.g., C₂-C₆ for straight chain, C₃-C₆ for branched chain). The term “C₂-C₆” includes alkynyl groups containing two to six carbon atoms.

“Substituted alkynyls” refers to alkynyl moieties having substituents replacing a hydrogen on one or more hydrocarbon backbone carbon atoms. Such substituents can include alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, or heterocyclyl.

Unless the number of carbons is otherwise specified, “lower alkyl” includes an alkyl group, as defined above, but having from one to ten, more preferably from one to six, carbon atoms in its backbone structure. “Lower alkenyl” and “lower alkynyl” have corresponding chain lengths, e.g., 2-5 carbon atoms.

“Acyl” includes compounds and moieties which contain the acyl radical (CH₃CO—) or a carbonyl group. “Substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Acylamino” includes moieties wherein an acyl moiety is bonded to an amino group. For example, the term includes alkylcarbonylamino, arylcarbonylamino, carbamoyl and ureido groups. “Alkylamino” includes moieties wherein an alkyl moiety is bonded to an amino group; “dialkylamino”, “arylamino”, “diarylamino”, and “alkylarylamino” are analogously named. In some embodiments, “amino” may include acylamino and/or alkylamino groups.

“Alkoxyalkyl” includes moieties where an alkoxy group is bonded to an alkyl group; “alkoxyaryl”, “thioalkoxyalkyl”, “alkylaminoalkyl” and “alkylthioalkyl” are analogously named.

“Alkoxy” or “alkoxyl” includes alkyl, alkenyl, and alkynyl groups covalently linked to an oxygen atom. Examples of alkoxy or alkoxyl groups include methoxy, ethoxy, isopropyloxy, propoxy, butoxy, and pentoxy groups. In an embodiment, the alkoxy or alkoxyl group is substituted. Examples of substituted alkoxy or substituted alkoxyl groups include halogenated alkoxy groups. Substituted alkoxy groups can include alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acylamino, amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, or heterocyclyl substituents. Examples of halogen-substituted alkoxy groups include fluoromethoxy, difluoromethoxy, trifluoromethoxy, chloromethoxy, dichloromethoxy, and trichloromethoxy.

The terms “heterocycloalkyl”, “heterocyclyl” or “heterocyclic group” include closed ring structures, e.g., 3- to 10-, or 4- to 7-membered rings which include one or more heteroatoms. Heterocyclyl groups can be saturated or unsaturated and include pyrrolidine, oxolane, thiolane, piperidine, piperizine, morpholine, lactones, lactams such as azetidinones and pyrrolidinones, sultams, sultones, and the like. Heterocyclic groups can have aromatic character such as pyrrole and furan. Heterocyclic groups includes fused ring structures such as quinoline and isoquinoline. Other examples of heterocyclic groups include pyridine and purine. Heterocyclic groups can also be substituted at one or more constituent atoms with, for example, a halogen, a lower alkyl, a lower alkenyl, a lower alkoxy, a lower alkylthio, a lower alkylamino, a lower alkylcarboxyl, a nitro, a hydroxyl, —CF₃, —CN, or the like. Heterocycloalkyl, heterocyclyl, or heterocyclic groups include spirocyclic groups.

Heterocyclic rings may be substituted at one or more positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino, acylamino, amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. In an embodiment, heterocyclic rings do not include bridged rings.

The term “heterocycloalkylene,” unless indicated otherwise, means the divalent radical of a closed ring structure, e.g., 3- to 10-, or 4- to 7-membered rings which include one or more heteroatoms. Heterocycloalkylene may be optionally substituted as described for heterocyclyl.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or heteroatoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

“Heteroatom” includes atoms of any element other than carbon or hydrogen. Examples of heteroatoms include nitrogen, oxygen, sulfur and phosphorus.

“At least partially aromatic bicyclic ring system”, means a bicyclic ring system where either or both of the rings forming the bicycle are aromatic.

It will be noted that the structure of some of the compounds of the invention includes asymmetric carbon atoms. It is to be understood accordingly that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

“Contacting” refers to the bringing together of indicated moieties in an in vitro or in vivo system. For example, “contacting” a chemokine receptor with a compound of the invention includes the administration of a compound of the invention to an individual or patient, such as a human, having a chemokine receptor, as well as, for example, introducing a compound of the invention into a sample containing a cellular or purified preparation containing the chemokine receptor.

“Selective” means that a compound binds to or inhibits a chemokine receptor with greater affinity or potency, respectively, compared to at least one other chemokine receptor, or preferably compared to all other chemokine receptors of the same class (e.g., all the CC-type receptors). In some embodiments, the compounds of the invention have binding or inhibition selectivity for CCR2 over any other chemokine receptor. Selectivity can be at least about 10-fold, at least about 20-fold, at least about 50-fold, at least about 100-fold, at least about 200-fold, at least about 500-fold or at least about 1000-fold. Binding affinity and inhibitor potency can be measured according to routine methods in the art.

An “anionic group,” as used herein, refers to a group that is negatively charged at physiological pH. Preferred anionic groups include carboxylate, sulfate, sulfonate, sulfinate, sulfamate, tetrazolyl, phosphate, phosphonate, phosphinate, or phosphorothioate or functional equivalents thereof “Functional equivalents” of anionic groups are intended to include bioisosteres, e.g., bioisosteres of a carboxylate group. Bioisosteres encompass both classical bioisosteric equivalents and non-classical bioisosteric equivalents. Classical and non-classical bioisosteres are known in the art (see, e.g., Silverman, R. B. The Organic Chemistry of Drug Design and Drug Action, Academic Press, Inc.: San Diego, Calif., 1992, pp. 19-23).

“Individual,” “patient,” or “subject” are used interchangeably and include to any animal, including mammals, preferably mice, rats, other rodents, rabbits, dogs, cats, swine, cattle, sheep, horses, or primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the invention is desirably a mammal in whom modulation of chemokine receptor activity is desired. “Modulation” includes antagonism (e.g., inhibition), agonism, partial antagonism and/or partial agonism. In some embodiments, compounds of the invention are antagonists (e.g., inhibitors) of chemokine receptors.

In the present specification, the term “therapeutically effective amount” means the amount of the subject compound that will elicit the biological or medical response of a tissue, system, animal or human that is being sought by the researcher, veterinarian, medical doctor or other clinician. The compounds of the invention are administered in therapeutically effective amounts to treat a disease, e.g., as rheumatoid arthritis. A therapeutically effective amount of a compound is that amount which results in the inhibition of one or more of the processes mediated by the binding of a chemokine to a receptor such as CCR2 in a subject with a disease associated with aberrant leukocyte recruitment and/or activation. Typical examples of such processes include leukocyte migration, integrin activation, transient increases in the concentration of intracellular free calcium and granule release of proinflammatory mediators. Alternatively, a therapeutically effective amount of a compound is the quantity required to achieve a desired therapeutic and/or prophylactic effect, such as an amount which results in the prevention of or a decrease in the symptoms associated with a disease associated with aberrant leukocyte recruitment and/or activation.

### EXPERIMENTAL SECTION

The invention now being generally described, will be more readily understood by reference to the following examples, which are included merely for purposes of illustration of certain aspects and embodiments of the present invention, and are not intended to limit the invention.

Provided below are representative procedures for making compounds encompassed by formulae Ia and Ib:

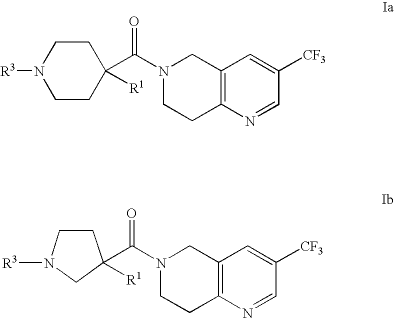

### A: General Procedure of Alkylation of Ester (Step 1 of Scheme 1 or Scheme 2)

To a solution of Boc-protected piperidine-4-carboxylate or pyrrolidine-3-carboxylate (1 eq.) cooled at −78° C. in tetrahydrofuran (THF) was added a solution of freshly prepared lithium diisopropylamide (LDA) (1 eq.). The solution was allowed to stir at −78° C. for 15 min and at 0° C. for 45 min. The appropriate alkyl halide was added and stirring was continued overnight. The crude product was purified by silica chromatography on an ISCO system (5-10% EtOAc/hexanes) to collect compounds of formula IIa or IIb in 50-75% yield.

### B: General Procedure of Saponification (Step 2 of Scheme 1 or Scheme 2)

Ester IIa or IIb (1 eq.) was heated with LiOH or KOH (10 eq.) in a mixture of MeOH/H₂O/THF (2.5/2.5/1.0) at 90° C. for 2-16 h. The solvent was removed under vacuum. The residue was washed with ethyl acetate to remove unreacted ester. The separated aqueous layer was acidified to pH 4 with 1M HCl, and then extracted with ethyl acetate (×3). The product of formula IIIa or IIIb was collected after solvent removal and/or purification by silica chromatography in 5% MeOH/DCM.

### C: General Procedure for Amide Formation (Step 1 and 2 in Scheme 4 and 5)

To acid IIa or IIb (1 eq.) in dichloromethane (DCM) was added 2M oxalyl chloride or SOCl₂ in DCM solution (3 eq.) and a few drops of dimethylformamide (DMF). The mixture was stirred at RT for 2 h and concentrated to under reduced pressure. The product was added to a solution of IVa (1 eq.) in DCM and triethyl amine (2.6 eq.) at 0° C. The mixture was stirred at 0° C. for 1 h, diluted with DCM and washed with sodium bicarbonate solution and water. The solution was dried over sodium sulfate and purified by silica chromatography to collect the desired product.

### D: General Procedure for Removal of Boc Group (Step 4 of Scheme 1 or Scheme 2)

Boc-protected amine Va or Vb (1 eq.) was stirred in DCM at 0° C. and trifluoroacetic acid (TFA) was added slowly. The ice bath was immediately removed after the addition of trifluoracetic acid (TFA). The resulting solution was stirred at room temperature for 1.5 h then a small amount of isopropyl alcohol was added. Concentration of the solution under reduced pressure gave the unprotected amine VIa or VIb as the trifluoroacetic acid salt, which was used without further purification in the next step. Compound VIa or VIb can be converted to a free base through a standard basic work-up (sodium bicarbonate solution).

### E: General Procedure for Reductive Amination (Step 5 of Scheme 1 or Scheme 2)

A mixture of aldehyde or ketone VII (1 eq.), acetic acid (1.5 eq.) and amine VI (1.2-1.5 eq.) in DCM/MeOH (1:2) was stirred at room temperature for 1 h. Sodium triacetoxyborohydride (2-3 eq.) was added and the reaction mixture was stirred for 16 h at room temperature. After concentration of solvent under reduced pressure, the resulting residue was dissolved in ethyl acetate, then washed with water and brine. The organic extract was dried, filtered and concentrated. The crude product was purified either by silica chromatography on an ISCO system or by reverse phase preparative HPLC to yield the desired final product I with purity greater than 95%.

F: General Procedure of Alkylation with Alkyl or Benzyl Halides (Step 5 of Scheme 1 or Scheme 2)

A mixture of piperidine or pyrrolidine intermediate VI (1 eq.), alkyl/benzyl halide VIII (1.2 eq.), diisopropylethylamine (1.5 eq.) and potassium carbonate (2.5 eq.) in DMF was irradiated in a microwave instrument at 110° C. for ˜20-30 min (Personal Chemistry Emrys™ Optimizer microwave reactor). The reaction mixture was cooled and diluted with ethyl acetate. The combined organic layers were washed with brine (×3), then dried over sodium sulfate, filtered and concentrated under reduced pressure. The crude product was purified either by column chromatography on ISCO system (the final product as free base) or by reverse phase preparative HPLC to yield the desired final product I as trifluoroacetic salt with purity greater than 95%.

### G: General Procedure of Benzylic Bromination (Scheme 3)

To a solution of 1,7-dimethylquinolin-2(1H)-one (Seq.) in 20 mL of CCl₄ were added azobis(isobutyronitrile) (AIBN) (1 eq.) and N-bromosuccinamide (1 eq.). The solution was stirred at 80° C. for 6 h. The product was extracted with DCM, washed with water and dried over sodium sulfate. The product was evaporated to dryness and purified by silica chromatography to give the desired product.

### H: General Procedure for Reductive Amination (Step 4 of Scheme 6)

To VIc (1 eq.), appropriate ketone (1 eq.) and Ti(OiPr)₄ (1.25 eq.) in THF were irradiated in a Microwave instrument at 100° C. for 30 min (Personal Chemistry Emrys™ Optimizer microwave reactor). The reaction mixture was cooled and NaBH(OAc)₃ (3 eq.) added. The mixture was stirred at room temperature overnight. The solvent was removed in vacuo. The residue was dissolved in ethyl acetate and washed with sat. sodium bicarbonate. The organic layer was washed with brine, then dried over sodium sulfate, filtered and concentrated under reduced pressure. The crude product was purified by reverse phase preparative HPLC to yield the desired final product as trifluoroacetic salt with purity greater than 95%

### Intermediate IIb

### 1-t-Butyl 3-methyl 3-(cyclopropylmethyl)pyrrolidine-1,3-dicarboxylate

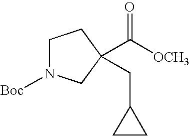

The title compound was prepared according to general procedure A described in connection with Scheme 2. ¹H NMR (400 MHz, CDCl₃): δ 3.93-3.77 (m, 1H), 3.72 (s, 3H), 3.45-3.22 (m, 3H), 2.45-2.32 (m, 1H), 1.89-1.79 (m, 1H), 1.65-1.59 (m, 2H), 1.48 (s, 9H), 0.67-0.57 (m, 1H), 0.48-0.42 (m, 2H), 0.06-0.01 (m, 2H); MS (ESI) m/z: Calculated for C₁₅H₂₅NO₄: 283.2; found: 306 (M+Na)⁺.

### Intermediate IIIb

### 1-(t-Butoxycarbonyl)-3-(cyclopropylmethyl)pyrrolidine-3-carboxylic acid

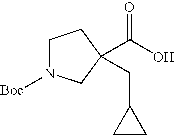

The title compound was prepared according to general procedure B described in connection with Scheme 2. 1-t-Butyl 3-methyl 3-(cyclopropylmethyl)pyrrolidine-1,3-dicarboxylate (1.00 g, 3.5 mmol.) was dissolved in 2 mL MeOH and a solution KOH (0.59 g, 10.6 mmol.) in 2 mL H₂O was added. The reaction mixture was microwaved at 130° C. for 30 min. The methanol was evaporated and 10 mL of 10% KHSO₄ solution was added to the residue. The acid was extracted with EtOAc (2×20 mL). The combined organic fractions were dried over Na₂SO₄; evaporation of solvent gave 0.98 g of the desired product as orange oil which crystallized on standing (quantitative yield). ¹H NMR (400 MHz, CDCl₃): δ 3.81-3.93 (m, 1H), 3.36-3.44 (m, 2H), 2.28 (d, 1H), 2.33-2.46 (m, 1H), 1.83-1.91 (m, 1H), 1.60-1.70 (m, 2H), 1.45 (s, 9H), 0.62-0.75 (m, 1H), 0.42-0.50 (m, 2H), 0.05-0.12 (m, 2H); MS (ESI) m/z: Calculated for C₁₄H₂₃NO₄: 269.2; found: 292 (M+Na)⁺.

### Intermediate Vb

### tert-butyl 3-(cyclopropylmethyl)-3-(3-(trifluoromethyl)-5,6,7,8-tetrahydro-1,6-naphthyridine-6-carbonyl)pyrrolidine-1-carboxylate

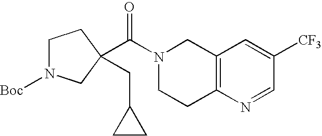

The title compound was prepared according to general procedure C described in connection with Scheme 5. To a solution of 1-(t-butoxycarbonyl)-3-(cyclopropylmethyl)pyrrolidine-3-carboxylic acid IIIb (300 mg, 1.11 mmol) in 16 mL of CH₂Cl₂ was added thionyl chloride (0.24 mL, 3.34 mmol) and two drops of DMF. The mixture was stirred for ˜1-2 h at room temperature. After removal of solvent, the residue was redissolved in CH₂Cl₂ (13 mL), followed by the addition of 3-(trifluoromethyl)-5,6,7,8-tetrahydro-1,6-naphthyridine hydrochloride (IVa) (320 mg, 1.16 mmol) and triethyl amine (0.78 mL, 5.5 mmol). LC-MS analysis indicated the completion of the reaction after one hour stirring at RT. The mixture was washed with water (×2) and brine (×3). After concentration of solvent, the crude residue was purified by flash chromatography on silica gel to give 300 mg (60% yield) of the desired product. MS (ESI) m/z: Calculated for C₂₃H₃₀F₃N₃O₃: 453.2; found: 453.7 (M+Na)⁺.

### Intermediate VIb

### (3-(Cyclopropylmethyl)pyrrolidin-3-yl)(3-(trifluoromethyl)-7,8-dihydro-1,6-naphthyridin-6(5H)-yl)methanone

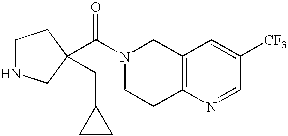

The title compound was prepared according to general procedure D described in connection with Scheme 5. tert-Butyl 3-(cyclopropylmethyl)-3-(3-(trifluoromethyl)-5,6,7,8-tetrahydro-1,6-naphthyridine-6-carbonyl)pyrrolidine-1-carboxylate was dissolved in 8 mL of CH₂Cl₂ and 2 mL of TFA was then added to the solution at RT. The reaction mixture was stirred for 4 h and the solvent was evaporated. The desired product was obtained as TFA salt without further application for next step. ¹H NMR (400 MHz, CDCl₃): δ 8.78 (s, 1H), 7.82-7.78 (m, 1H), 6.86 (br s, 1H), 4.88 (dd, 2H), 3.98 (m, 3H), 3.42 (d, 1H), 3.33 (m, 1H), 3.22 (br s, 2H), 2.63 (m, 1H), 2.20 (m, 1H), 2.01-1.73 (m, 3H), 0.68 (s, 1H), 0.53 (br s, 2H), 0.066 (m, 2H); MS (ESI) m/z: Calculated for C₁₈H₂₂F₃N₃O: 353.2; found 354.1 (M+H)⁺.

### Intermediate VIIIa

### 7-(Bromomethyl)-1-methylquinolin-2(1H)-one

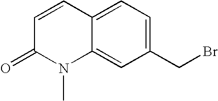

The title compound was prepared according to general procedure G described in connection with Scheme 3. 7-Methylquinoline (3 g, 20.95 mmol) was dissolved in 20 mL methyliodide and stirred for 3 h at RT. The reaction mixture was dissolved in 30 mL of acetonitrile and KMnO₄ (6.62 g, 41.90 mmol) was added portionwise. The reaction mixture was stirred for 1 h while violet changed to brown color. Saturated solution of sodiummetabisulfite solution was carefully added. Then 10% HCl was added and extracted with dichloromethane. The solvent was dried over Na₂SO₄ and evaporated by rotary evaporator. The crude product was purified by ISCO flash chromatography (1-4% CH₂Cl₂/CH₃OH)) to yield 2.75 g (76%) of pure product. ¹H NMR (400 MHz, CDCl₃): δ 7.63 (d, 1H), 7.43 (d, 1H), 7.20 (s, 1H), 7.12 (d, 1H), 6.61 (d, 1H), 3.75 (s, 3H), 2.57 (s, 3H); MS (ESI) m/z: Calculated for C₁₁H₁₁NO: 173.21; found: 173.2 (M)⁺.

To a solution of 1,7-dimethylquinolin-2(1H)-one (1.2 g, 6.92 mmol) in 20 mL of CCl₄ were added AIBN (0.227 g, 1.38 mmol) and N-bromosuccinamide (1.23 g, 6.92 mmol). The solution was stirred at 80° C. for 6 h. The product was extracted with dichloromethane and washed with water and dried over Na₂SO₄. The product was evaporated to dryness and purified by ISCO system (1-4% CH₂Cl₂/CH₃OH) to give 1.25 g (72%). ¹H NMR (400 MHz, CDCl₃): δ 7.64 (d, 1H), 7.53 (d, 1H), 7.38 (s, 1H), 7.27 (d, 1H), 6.73 (d, 1H), 4.60 (s, 2H), 3.73 (s, 3H); MS (ESI) m/z: Calculated for C₁₁H₁₀BrNO: 252.11; found: 252.1 (M)⁺.

A series of compounds were synthesized based on the procedures described above. The structures and MS-characteristics, if available, of the compounds are summarized in Table 1:

TABLE 1

| Example | | | Calc | Synthetic |
|---|---|---|---|---|
| No. | Structure | m/z | MW | Methods |
| 1 | 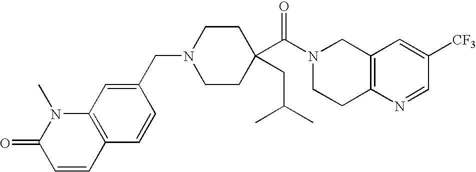 | 541.2 | 540.3 | Scheme 4 |
| 2 | 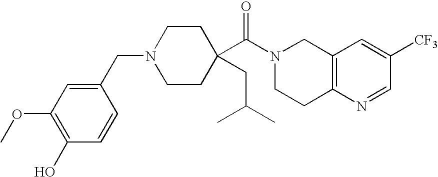 | 506.1 | 505.3 | Scheme 4 |
| 3 | 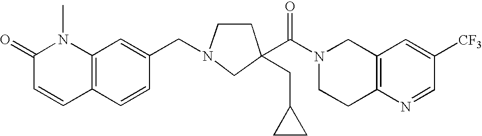 | 525.2 | 524.2 | Scheme 5 |
| 4, 5 | 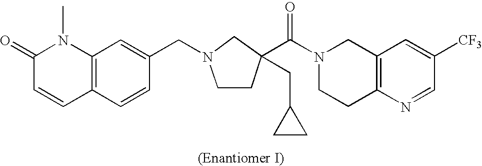 | 525.2 | 524.2 | Scheme 5 |
| 4, 5 | 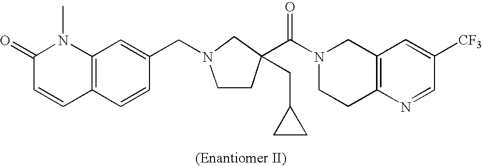 | 525.2 | 524.2 | Scheme 5 |
| 6 | 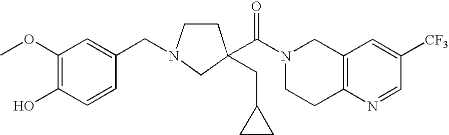 | 490.1 | 489.2 | Scheme 5 |
| 7 | 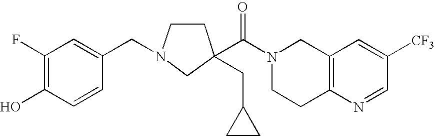 | 478.1 | 477.2 | Scheme 5 |
| 8 | 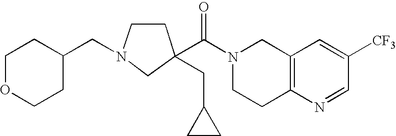 | 452.2 | 451.2 | Scheme 5 |
| 9 | 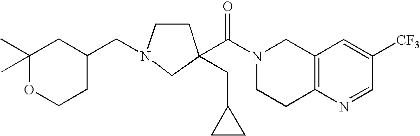 | 480.2 | 479.3 | Scheme 5 |
| 10 | 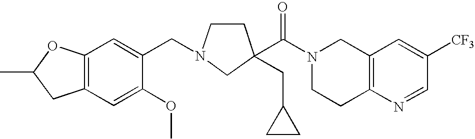 | 530.2 | 529.3 | Scheme 5 |
| 11 | 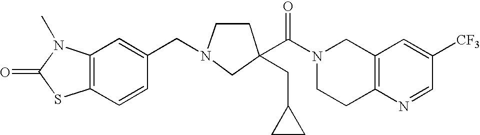 | 531.1 | 530.2 | Scheme 5 |
| 12 | 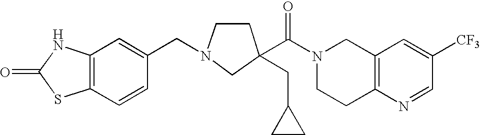 | 517.1 | 516.2 | Scheme 5 |
| 13 | 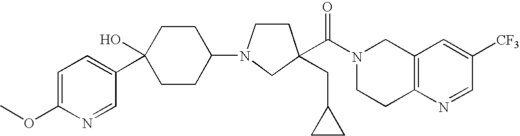 | 559.2 | 558.3 | Scheme 5 |
| 14 | 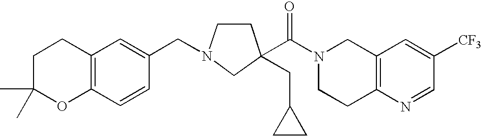 | 528.1 | 527.3 | Scheme 5 |
| 15 | 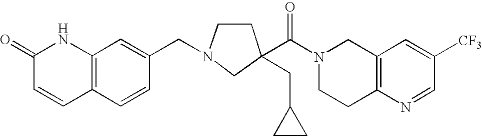 | 511.2 | 510.2 | Scheme 7 |
| 16 | 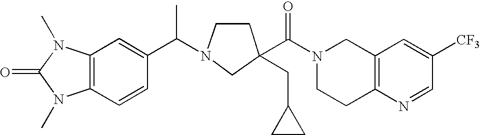 | 542.4 | 541.3 | Scheme 6 |
| 17 | 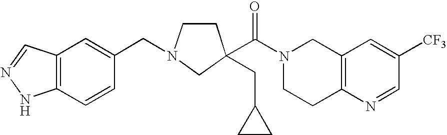 | 484 | 483.2 | Scheme 5 |
| 18 | 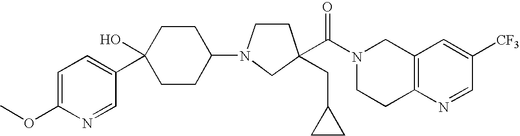 | 559 | 558.3 | Scheme 8 |
| 19 | 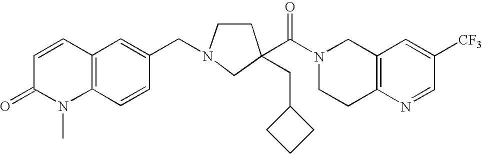 | 539.4 | 538.3 | Scheme 5 |
| 20 | 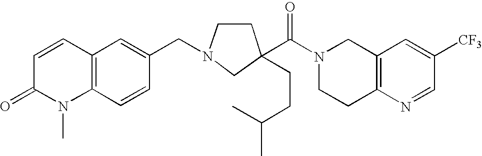 | 541.4 | 540.3 | Scheme 5 |
| 21 | 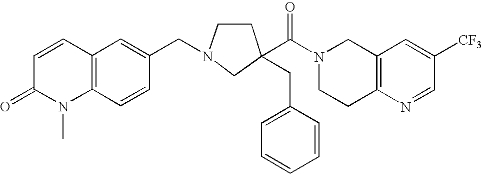 | 561.3 | 560.2 | Scheme 5 |
| 22 | 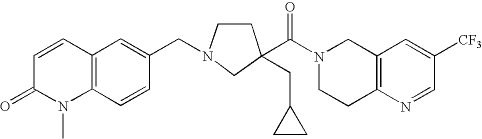 | 525.1 | 524.2 | Scheme 5 |
| 23 | 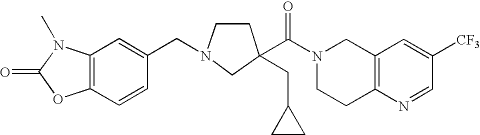 | 515.1 | 514.2 | Scheme 5 |
| 24 | 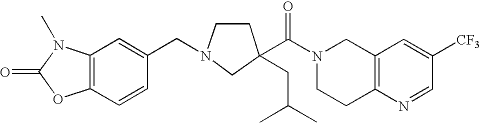 | 517.2 | 516.2 | Scheme 5 |
| 25 | 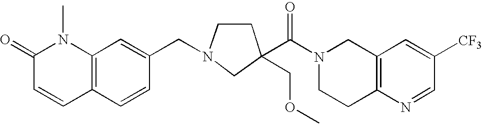 | 515 | 514.2 | Scheme 5 |
| 26, 27 | 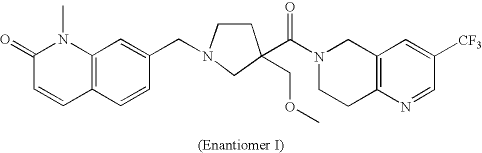 | 515 | 514.2 | Scheme 5 |
| 26, 27 | 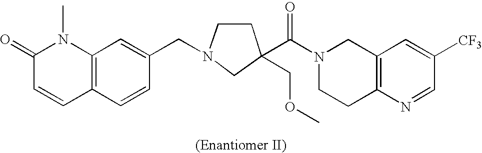 | 515 | 514.2 | Scheme 5 |
| 28 | 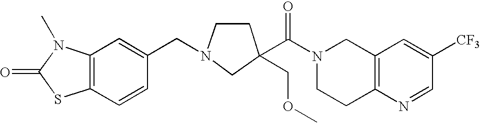 | 521.1 | 520.2 | Scheme 5 |
| 29 | 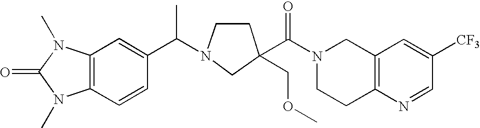 | 532.0 | 531.3 | Scheme 6 |
| 30, 31 | 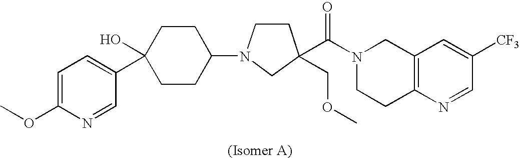 | 549.2 | 548.3 | Scheme 8 |
| 30, 31 | 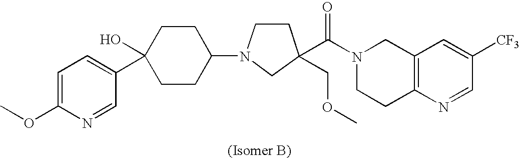 | 549.2 | 548.3 | Scheme 8 |
| 32 | 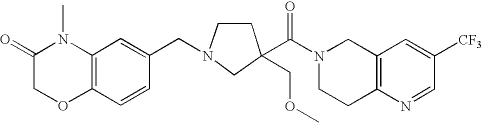 | 519.3 | 518.2 | Scheme 5 |
| 33 | 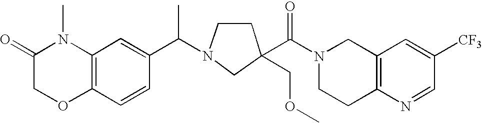 | 533.2 | 532.2 | Scheme 6 |
| 34 | 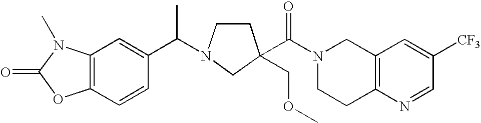 | 519.2 | 518.2 | Scheme 6 |
| 35 | 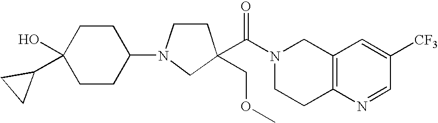 | 482.2 | 481.3 | Scheme 8 |
| 36 | 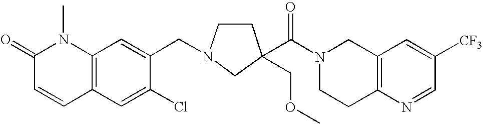 | 549 | 548.2 | Scheme 5 |
| 37 | 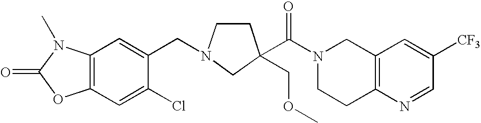 | 539 | 538.2 | Scheme 5 |
| 38 | 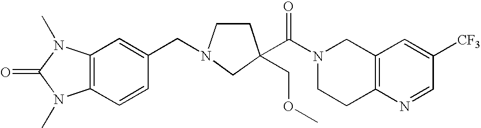 | 518 | 517.2 | Scheme 5 |
| 39 | 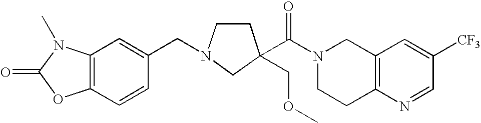 | 505.1 | 504.2 | Scheme 5 |
| 40, 41 | 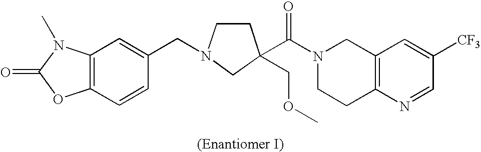 | 505.1 | 504.2 | Scheme 5 |
| 40, 41 | 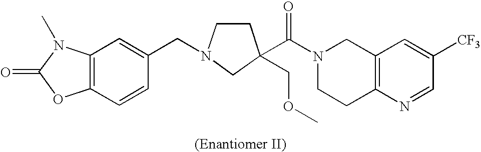 | 505.1 | 504.2 | Scheme 5 |
| 42 | 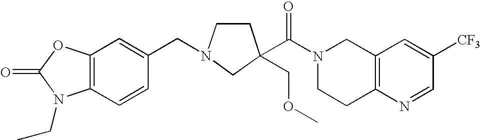 | 519.2 | 518.2 | Scheme 5 |
| 43 | 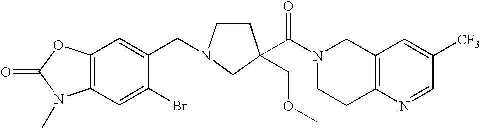 | 583.2 | 582.11 | Scheme 5 |
| 44 | 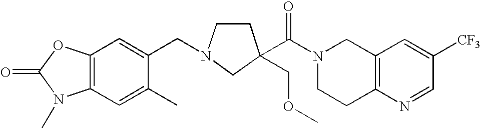 | 519.2 | 518.2 | Scheme 5 |
| 45 | 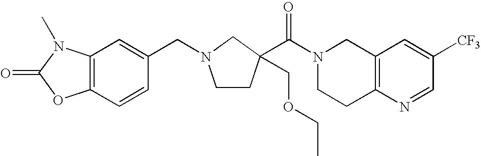 | 519.2 | 518.53 | Scheme 5 |
| 46 | 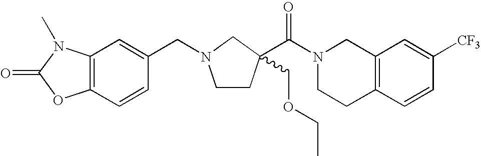 | 504.2 | 503.51 | Scheme 5 |
| 47 | 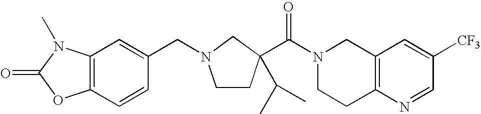 | 503.1 | 502.53 | Scheme 5 |
| 48 | 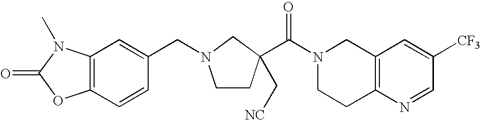 | 500.3 | 499.48 | Scheme 5 |
| 49 | 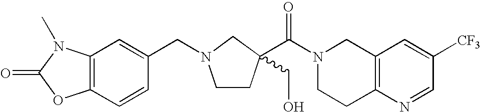 | 491.2 | 490.47 | Scheme 5 |
| 50, 51 (Isomer I) | 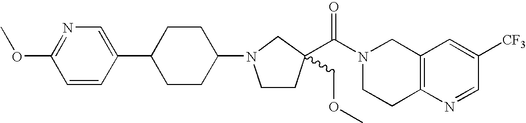 | 533.2 | 532.60 | Scheme 8 |
| 50, 51 (Isomer II) | 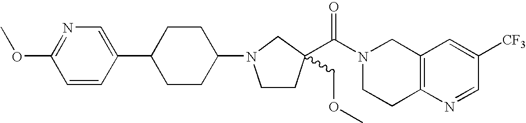 | 533.2 | 532.60 | Scheme 8 |
| 52, 53 (Isomer II) | 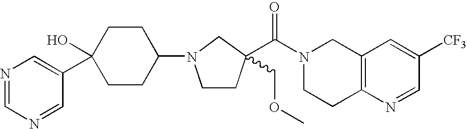 | 520.2 | 519.26 | Scheme 8 |
| 52, 53 (Isomer II) | 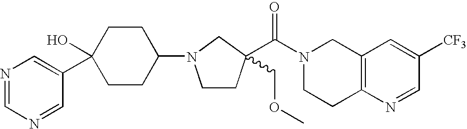 | 520.2 | 519.26 | Scheme 8 |
| 54, 55 (Isomer II) | 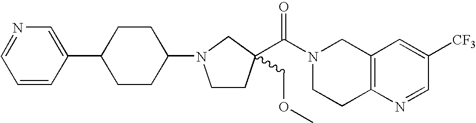 | 504.3 | 503.55 | Scheme 8 |
| 54, 55 (Isomer II) | 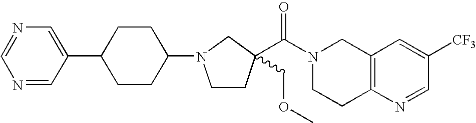 | 504.3 | 503.55 | Scheme 8 |
| 56, 57 (Isomer I) | 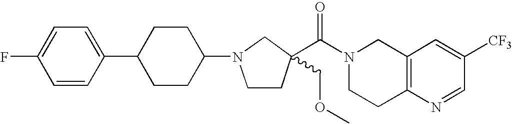 | 520.2 | 519.57 | Scheme 9 |
| 56, 57 (Isomer II) | 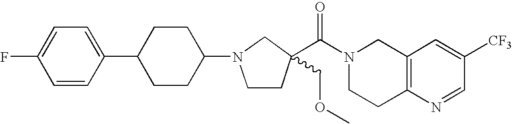 | 520.2 | 519.57 | Scheme 9 |
| 58 | 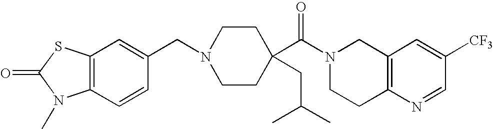 | 547.0 | 546.65 | Scheme 4 |
| 59 | 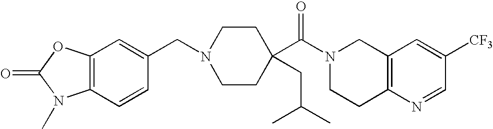 | 531.2 | 530.58 | Scheme 4 |
| — | 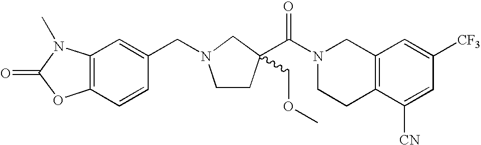 | — | — | Scheme 5 |
| — | 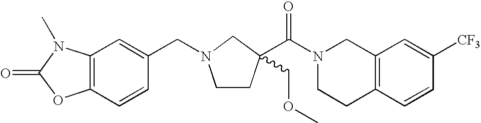 | — | — | Scheme 5 |
| — | 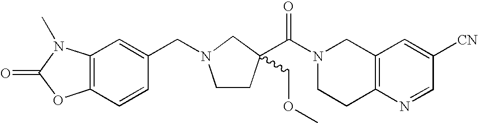 | — | — | Scheme 5 |
| — | 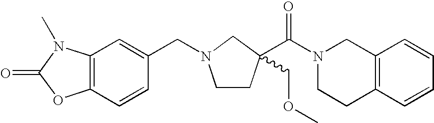 | — | — | Scheme 5 |
| — | 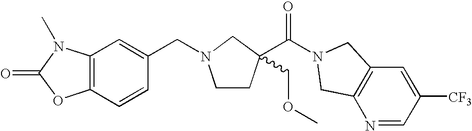 | — | — | Scheme 5 |
| — | 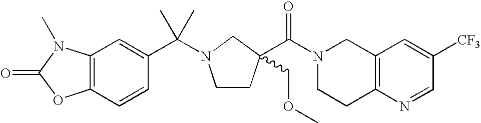 | — | — | Scheme 6 |
| — | 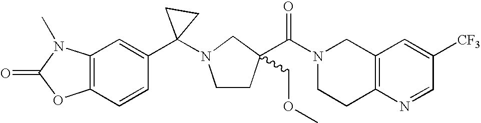 | — | — | Scheme 6 |
| — | 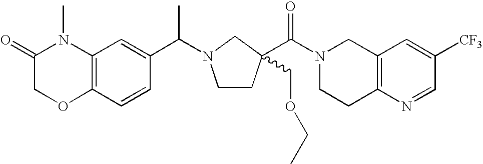 | — | — | Scheme 6 |
| — | 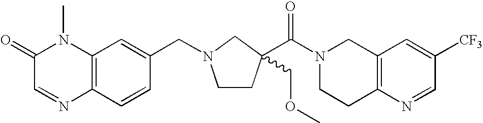 | — | — | Scheme 5 |
| — | 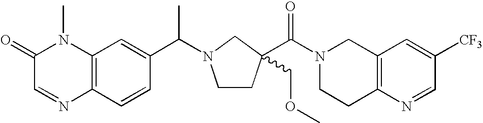 | — | — | Scheme 6 |
| — | 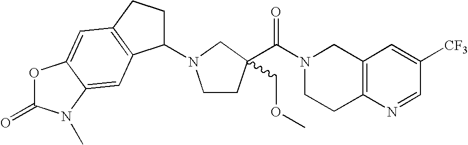 | — | — | Scheme 5 |
| — | 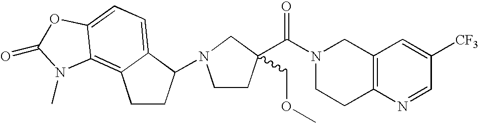 | — | — | Scheme 5 |
| — | 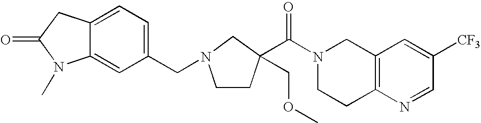 | — | — | Scheme 5 |
| — | 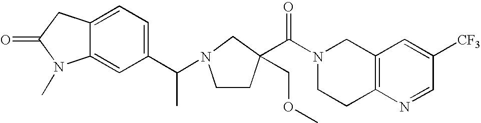 | — | — | Scheme 6 |
| — | 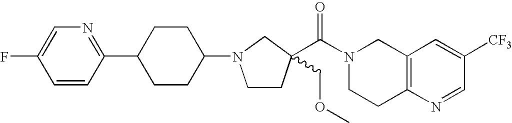 | — | — | Scheme 8 |
| — | 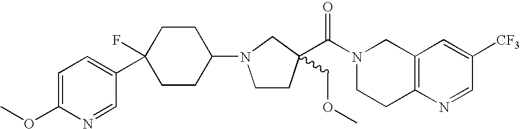 | — | — | Scheme 8 |
| — | 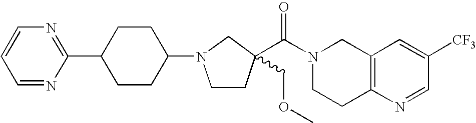 | — | — | Scheme 8 |
| — | 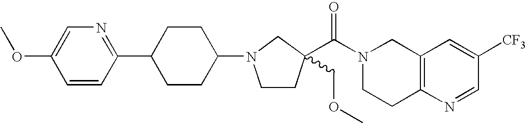 | — | — | Scheme 8 |
| — | 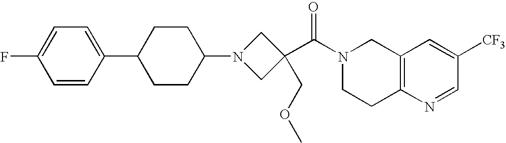 | — | — | Scheme 10 |
| — | 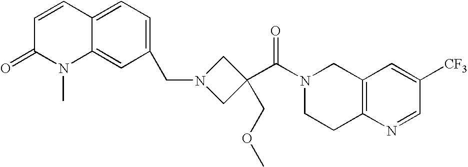 | — | — | Scheme 10 |
| — | 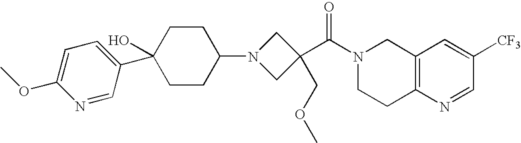 | — | — | Scheme 10 |
| — | 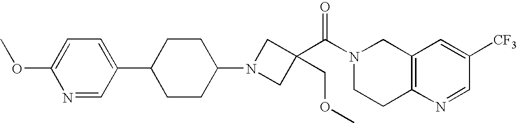 | — | — | Scheme 10 |

### Example 1

### 7-((4-Isobutyl-4-(3-(trifluoromethyl)-5,6,7,8-tetrahydro-1,6-naphthyridine-6-carbonyl)piperidin-1-yl)methyl)-1-methylquinolin-2(1H)-one

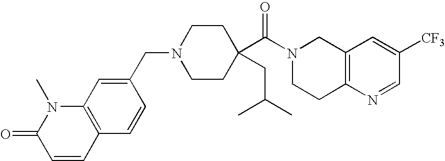

The title compound was prepared according to the general procedures described in Scheme 4: a mixture of (4-isobutylpiperidin-4-yl)(3-(trifluoromethyl)-7,8-dihydro-1,6-naphthyridin-6(5H)-yl)methanone VIa (56 mg, 0.094 mmol), intermediate VIIIa (25 mg, 0.098 mmol), diisopropylethylamine (70 μL, 0.39 mmol) and potassium carbonate (33 mg, 0.235 mmol) in DMF (2 mL) was irradiated in a Microwave instrument at 110° C. for 30 min (Personal Chemistry Emrys™ Optimizer microwave reactor). The reaction mixture was cooled and diluted with ethyl acetate. The organic layer was washed with brine (×3), then dried over Na₂SO₄, filtered and concentrated under reduced pressure. The crude product was purified by reverse phase preparative HPLC to yield the desired final product as trifluoroacetic salt with purity greater than 95% (36.2 mg, 71% yield): ¹H NMR (400 MHz, CD₃OD): δ 8.72 (br s, 1H), 8.07 (s, 1H), 7.94 (d, 1H), 7.79 (d, 1H), 7.72 (s, 1H), 7.39 (d, 1H), 6.74 (d, 1H), 4.96 (m, 2H), 4.41 (s, 2H), 4.04 (s, 2H), 3.75 (s, 3H), 3.46 (d, 2H), 3.11 (m, 4H), 2.69-2.65 (m, 2H), 1.70 (m, 5H), 0.81 (m, 6H); MS (ESI) m/z: Calculated for C₃₀H₃₅F₃N₄O₂: 540.3; found: 541.2 (M+H)⁺.

### Example 2

### (1-(4-Hydroxy-3-methoxybenzyl)-4-isobutylpiperidin-4-yl)(3-(trifluoromethyl)-7,8-dihydro-1,6-naphthyridin-6(5H)-yl)methanone

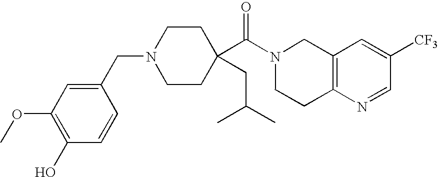

The title compound was prepared according to general procedures described in Scheme 4 (2.0 mg, 4% yield): ¹H NMR (400 MHz, CD₃OD): δ 8.72 (s, 1H), 8.08 (s, 1H), 7.02 (s, 1H), 6.48 (d, 1H), 6.90-6.81 (m, 2H), 4.13 (s, 2H), 4.02 (d, 2H), 3.89 (d, 2H), 3.86 (s, 3H), 3.40 (d, 2H), 3.10 (m, 2H), 3.02 (m, 2H), 2.65 (d, 2H), 1.67 (m, 5H), 0.82 (m, 6H); MS (ESI) m/z: Calculated for C₂₇H₃₄F₃N₃O₃: 505.3; found: 506.1 (M+H)⁺.

### Example 3

### 7-((3-(Cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one

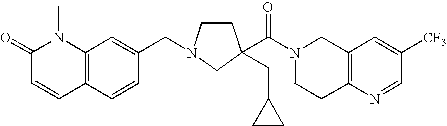

The title compound was prepared according to general procedures described in Scheme 5 (9.1 mg, 39% yield): ¹H NMR (400 MHz, CD₃OD): δ 8.71 (s, 1H), 8.05 (s, 1H), 7.95 (d, 1H), 7.82 (d, 1H), 7.79 (s, 1H), 7.46 (d, 1H), 6.75 (d, 1H), 4.85 (m, 2H), 4.57 (q, 2H), 3.96 (br s, 2H), 3.78 (s, 3H), 3.60 (br s, 1H), 3.44 (br s, 1H), 3.12 (s, 2H), 2.55 (m, 2H), 2.00 (s, 2H), 1.84 (m, 2H), 0.54 (s, 1H), 0.42 (s, 2H), 0.05 (m, 2H); MS (ESI) m/z: Calculated for C₂₉H₃₁F₃N₄O₂: 524.2; found: 525.2 (M+H)⁺.

### Examples 4 and 5

### (S)-7-((3-(Cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one and (R)-7-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one

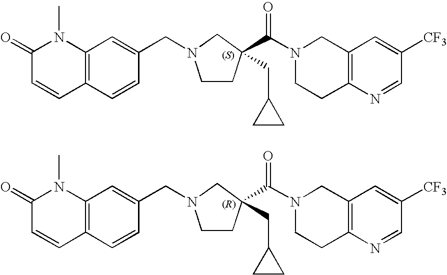

The racemic mixture (ca. 1:1 ratio) was separated into the two enantiomers by normal phase preparative HPLC using a chiral column, yielding enantiomer I (>95% ee; eluented at 4.79 min), and enantiomer II (>95% ee; eluented at 8.03 min).

### Example 6

### (1-(4-Hydroxy-3-methoxybenzyl)-3-(cyclopropylmethyl)pyrrolidin-3-yl)(3-(trifluoromethyl)-7,8-dihydro-1,6-naphthyridin-6(5H)-yl)methanone

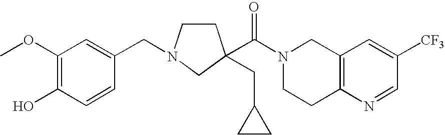

The title compound was prepared according to general procedures described in Scheme 5 (5.6 mg, 7% yield): ¹H NMR (400 MHz, CD₃OD): δ 8.71 (s, 1H), 8.05 (s, 1H), 7.08 (s, 1H), 6.95 (d, 1H), 6.86 (d, 1H), 4.82 (m, 2H), 4.59 (d, 1H), 4.28 (q, 2H), 3.97-3.92 (m, 2H), 3.89 (s, 3H), 3.54 (m, 2H), 3.18-3.12 (m, 3H), 2.49 (m, 2H), 2.00 (m, 1H), 1.80 (dd, 2H), 0.53 (d, 1H), 0.433 (d, 2H), 0.02 (m, 2H); MS (ESI) m/z: Calculated for C₂₆H₃₀F₃N₃O₃: 489.2; found: 490.1 (M+H)⁺.

### Example 7

### (3-(Cyclopropylmethyl)-1-(3-fluoro-4-hydroxybenzyl)pyrrolidin-3-yl)(3-(trifluoromethyl)-7,8-dihydro-1,6-naphthyridin-6(5H)-yl)methanone

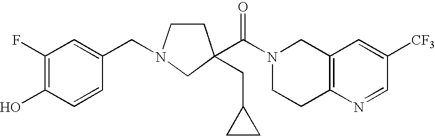

The title compound was prepared according to general procedures described in Scheme 5 (4.0 mg, 5% yield): ¹H NMR (400 MHz, CD₃OD): δ 8.08 (s, 1H), 7.27 (d, 1H), 7.15 (d, 1H), 7.00 (t, 1H), 4.49 (d, 1H), 4.29 (q, 2H), 3.98 (s, 2H), 3.54 (m, 2H), 3.32 (s, 3H), 3.22-3.15 (m, 2H), 2.51 (s, 1H), 1.83 (m, 2H), 1.31 (m, 2H), 0.53-0.44 (m, 3H), 0.05 (m, 2H); MS (ESI) m/z: Calculated for C₂₅H₂₇F₄N₃O₂: 477.2; found: 478.1 (M+H)⁺.

### Example 8

### (3-(Cyclopropylmethyl)-1-((tetrahydro-2H-pyran-4-yl)methyl)pyrrolidin-3-yl)(3-(trifluoromethyl)-7,8-dihydro-1,6-naphthyridin-6(5H)-yl)methanone

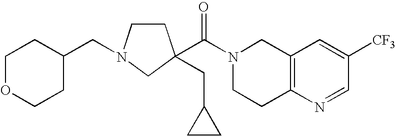

The title compound was prepared according to general procedures described in Scheme 5 (5.6 mg, 4% yield): ¹H NMR (400 MHz, CD₃OD): δ 8.72 (s, 1H), 8.06 (m, 1H), 4.84 (s, 1H), 4.71 (d, 1H), 3.98-3.94 (m, 4H), 3.64 (m, 1H), 3.48 (m, 2H), 3.12 (m, 6H), 2.48 (s, 1H), 2.13 (s, 2H), 1.86-1.80 (dd, 2H), 1.68 (d, 2H), 1.49-1.29 (m, 3H), 0.58-0.47 (m, 3H), 0.05 (m, 2H); MS (ESI) m/z: Calculated for C₂₄H₃₂F₃N₃O₂: 451.2; found: 452.2 (M+H)⁺.

### Example 9

### (3-(Cyclopropylmethyl)-1-((2,2-dimethyltetrahydro-2H-pyran-4-yl)methyl)pyrrolidin-3-yl)(3-(trifluoromethyl)-7,8-dihydro-1,6-naphthyridin-6(5H)-yl)methanone

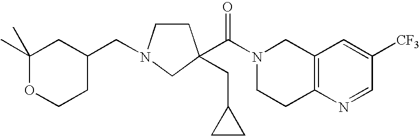

The title compound was prepared according to general procedures described in Scheme 5 (36.5 mg, 29.6% yield). ¹H NMR (400 MHz, MeOH-d₄): δ 9.04 (s, 1H), 8.55 (s, 1H), 4.66 (d, 1H), 3.99 (m, 2H), 3.75 (m, 3H), 3.12 (m, 4H), 2.80 (m, 1H), 2.48 (m, 1H), 2.32 (m, 2H), 2.04 (d, 1H), 1.75 (m, 4H), 1.24 (m, 10H), 0.55 (m, 3H), 0.10 (d, 2H); MS (ESI) m/z: Calculated for C₂₆H₃₆F₃N₃O₂: 479.3; found: 480.2 (M+H)⁺.

### Example 10

### (3-(Cyclopropylmethyl)-1-((5-methoxy-2-methyl-2,3-dihydrobenzofuran-6-yl)methyl)pyrrolidin-3-yl)(3-(trifluoromethyl)-7,8-dihydro-1,6-naphthyridin-6(5H)-yl)methanone

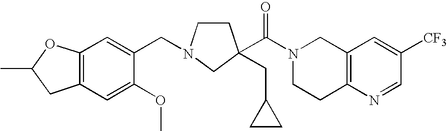

The title compound was prepared according to general procedures described in Scheme 5 (30.5 mg, 22.5% yield). ¹H NMR (400 MHz, MeOH-d₄): δ 8.71 (s, 1H), 8.06 (s, 1H), 7.02 (s, 1H), 6.76 (s, 1H), 4.47 (d, 1H), 4.32 (bs, 2H), 3.87 (m, 4H), 3.55 (m, 2H), 3.34 (m, 2H), 3.19 (d, 1H), 3.11 (m, 2H), 2.85 (m, 2H), 2.46 (m, 2H), 2.15 (m, 2H), 1.78 (m, 2H), 1.40 (d, 3H), 0.44 (m, 3H), 0.10 (m, 2H); MS (ESI) m/z: Calculated for C₂₉H₃₄F₃N₃O₃: 529.3; found: 530.2 (M+H)⁺.

### Example 11

### 5-((3-(Cyclopropylmethyl)-3-(3-(trifluoromethyl)-5,6,7,8-tetrahydro-1,6-naphthyridine-6-carbonyl)pyrrolidin-1-yl)methyl)-3-methylbenzo[d]thiazol-2(3H)-one

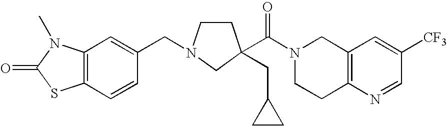

The title compound was prepared according to general procedures described in Scheme 5 (42.0 mg, 35.5% yield). ¹H NMR (400 MHz, CDCl₃): δ 8.71 (s, 1H), 7.69 (s, 1H), 7.35 (d, 1H), 7.12 (m, 2H), 4.79 (m, 1H), 3.83 (d, 2H), 3.66 (q, 2H), 3.47 (m, 3H), 3.27 (d, 1H), 3.13 (q, 2H), 2.97 (s, 1H), 2.69 (m, 2H), 2.59 (m, 1H), 2.42 (m, 1H), 1.96 (m, 1H), 1.82 (m, 1H), 1.68 (m, 2H), 0.59 (m, 2H), 0.41 (m, 2H); MS (ESI) m/z: Calculated for C₂₇H₂₉F₃N₄O₂S: 530.2; found: 531.1 (M+H)⁺.

### Example 12

### 5-((3-(Cyclopropylmethyl)-3-(3-(trifluoromethyl)-5,6,7,8-tetrahydro-1,6-naphthyridine-6-carbonyl)pyrrolidin-1-yl)methyl)benzo[d]thiazol-2(3H)-one

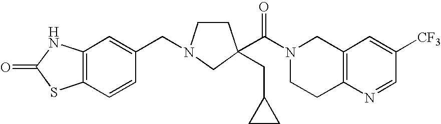

The title compound was prepared according to general procedures described in Scheme 5 (73.9 mg, 56.4% yield). ¹H NMR (400 MHz, CDCl₃): δ 8.71 (s, 1H), 7.69 (s, 1H), 7.30 (d, 1H), 7.17 (s, 1H), 7.09 (d, 1H), 4.79 (m, 2H), 3.92 (m, 1H), 3.80 (m, 1H), 3.62 (q, 2H), 3.26 (d, 1H), 3.11 (q, 2H), 2.71 (m, 2H), 2.59 (m, 1H), 2.42 (m, 1H), 2.08 (s, 1H), 1.95 (m, 1H), 1.81 (m, 1H), 1.67 (m, 1H), 0.59 (m, 1H), 0.40 (m, 2H), −0.07 (m, 2H); MS (ESI) m/z: Calculated for C₂₆H₂₇F₃N₄O₂S: 516.2; found: 517.1 (M+H)⁺.

### Example 13

### 3-(Cyclopropylmethyl)-1-(4-hydroxy-4-(6-methoxypyridin-3-yl)cyclohexyl)pyrrolidin-3-yl)(3-(trifluoromethyl)-7,8-dihydro-1,6-naphthyridin-6(5H)-yl)methanone

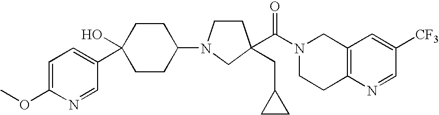

The title compound was prepared according to the same procedures as described for Example 30 (70.0 mg, 73.7% yield). ¹H NMR (300 MHz, CD₃Cl₃): δ 8.72 (s, 1H), 8.29 (d, 1H), 8.06 (s, 1H), 7.97 (dd, 1H), 6.93 (d, 1H), 4.60 (m, 2H), 3.96 (m, 7H), 3.72 (m, 1H) 3.38 (m, 1H), 3.13 (m, 4H), 2.50 (m, 4H), 2.20 (m, 1H), 2.0.7 (bs, 1H), 1.95-1.82 (m, 5H), 1.69 (m, 1H), 0.60 (bs, 1H), 0.46 (bs, 2H), 0.02 (bs, 2H); MS (ESI) m/z: Calculated for C₃₀H₃₇F₃N₄O₃: 558.3; found: 559.2 (M+H)⁺.

### Example 14

### (3-(Cyclopropylmethyl)-1-(2,2-dimethylchromoa-6-yl)methyl)pyrrolidin-3-yl)(3-(trifluoromethyl)-7,8-dihydro-1,6-naphthyridin-6(5H)-yl)methanone

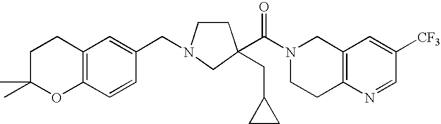

The title compound was prepared according to general procedures described in Scheme 5 (35 mg, 16% yield). ¹H NMR (400 MHz, CD₃OD): δ 8.73 (s, 1H), 8.08 (s, 1H), 7.08 (s, 1H), 7.25-7.19 (m, 1H), 6.80 (d, 1H), 4.60-3.90 (m, 6H), 3.62-3.01 (m, 4H), 2.90-2.20 (m, 8H), 1.80 (d, 2H), 1.36 (s, 6H), 0.61-0.02 (m, 5H); MS (ESI) m/z: Calculated for C₃₀H₃₆F₃N₃O₂: 527.3; found: 528.1 (M+H)⁺.

### Example 15

### 7-((3-(Cyclopropylmethyl)-3-(3-(trifluoromethyl)-5,6,7,8-tetrahydro-1,6-naphthyridin-6-carbonyl)pyrrolidin-1-yl)methyl)quinolin-2(1H)-one

The title compound was prepared via the following intermediates using the procedures described below.

### N-m-Tolylcinnamamide

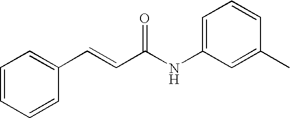

m-Toluidine (5 g, 46.64 mmol) and pyridine (3.77 mL, 46.64 mmol) were dissolved in dry dichloromethane (25 mL). To the reaction mixture, cinnamoyl chloride (7.72 g, 46.64 mmol) was added and stirred for 3 h at 0° C. The reaction mixture was extracted with dichloromethane washing with water and 2N HCl. The solvent was removed and the crude product was used in the next step without further purification.

### 7-Methylquinolin-2(1H)-one

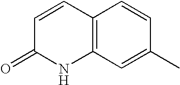

N-m-tolylcinnamamide (2 g, 0.834 mmol) and AlCl₃ (1.12 g, 0.834 mmol) were heated for 1 h at 100° C. Water was added and the solid was filtered to provide 1.2 g of crude product.

### 7-(Bromomethyl)quinolin-2(1H)-one

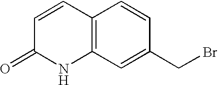

The title compound was prepared according to general procedures described in Scheme 3 (90 mg, 24% yield). ¹H NMR (400 MHz, CDCl₃): δ 7.64 (d, 1H), 7.55 (d, 1H), 7.38 (s, 1H), 7.28 (d, 1H), 6.73 (d, 1H), 4.60 (s, 2H); MS (ESI) m/z: Calculated for C₁₀H₈BrNO: 236.9; found: 238.1 (M+H)⁺.

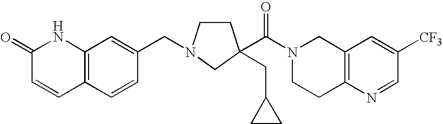

The title compound was prepared according to general procedures described in Scheme 5 (20 mg, 27% yield). ¹H NMR (400 MHz, CD₃OD): δ 8.76 (s, 1H), 8.07 (s, 1H), 8.01 (d, 1H), 7.80 (s, 1H), 7.51 (s, 1H), 7.40 (d, 1H), 6.78 (d, 1H), 4.70-4.40 (m, 6H), 4.00-3.01 (m, 4H), 2.60-2.55 (m, 2H), 1.96-1.91 (m, 2H), 1.80 (d, 2H), 0.60-0.02 (m, 5H); MS (ESI) m/z: Calculated for C₂₈H₂₉F₃N₄O₂: 510.2; found: 511.2 (M+H)⁺.

### Example 16

### 5-(1-(3-(Cyclopropylmethyl)-3-(3-(trifluoromethyl)-5,6,7,8-tetrahydro-1,6-naphthyridine-6-carbonyl)pyrrolidin-1-yl)ethyl)-1,3-dimethyl-1H-benzo[d]imidazol-2(3H)-one

The title compound was prepared via the intermediate shown below using the procedures described below.

### 5-Acetyl-1,3-dimethyl-1H-benzo[d]imidazol-2(3H)-one

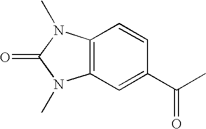

5-Acetyl-1H-benzo[d]imidazol-2(3H)-one (1 g, 5.67 mmol), methyl iodide (3.22 g, 22.71 mmol), and cesium carbonate (4.62 g, 14.19 mmol) were dissolved in DMF (2 mL). The reaction mixture was irradiated by microwave for 30 minutes at 100° C. The solvent was removed and extracted with dichloromethane. Purification by silica chromatography (ISCO) produced 5-acetyl-1,3-dimethyl-1H-benzo[d]imidazol-2(3H)-one (0.95 g, 82%). ¹H NMR (400 MHz, CDCl₃): δ 7.79 (d, 1H), 7.61 (s, 1H), 7.00 (d, 1H), 3.50 (s, 6H), 2.62 (s, 3H); MS (ESI) m/z: Calculated for C₁₁H₁₂N₂O₂: 204.1; found: 205.2 (M+H)⁺.

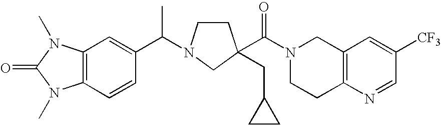

The title compound was prepared according to general procedures described in Scheme 6 using 1 equivalent of Ti(OiPr)₄ (14 mg, 4% yield). ¹H NMR (300 MHz, CDCl₃): δ 8.79 (s, 1H), 7.78 (s, 1H), 7.40-6.90 (m, 3H), 4.90-4.65 (m, 4H), 4.20-3.80 (m, 3H), 3.44 (s, 3H), 3.40 (s, 3H), 3.20-3.15 (m, 2H), 2.95-2.36 (m, 4H), 2.01-1.82 (m, 2H), 1.80 (d, 3H), 0.60-0.02 (m, 5H); MS (ESI) m/z: Calculated for C₂₉H₃₄F₃N₅O₂: 541.3; found: 542.4 (M+H)⁺.

### Example 17

### (1-((1H-Indazol-5-yl)methyl)-3-(cyclopropylmethyl)pyrrolidin-3-yl)(3-(trifluoromethyl)-7,8-dihydro-1,6-naphthyridin-6(5H)-yl)methanone

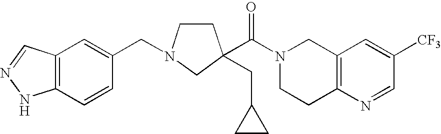

The title compound was prepared according to general procedures described in Scheme 5 (38 mg, 16% yield). ¹H NMR (400 MHz, DMSO-d₆): δ 13.28 (bs, 2H), 9.94 (bs, 1H), 9.77 (bs, 1H), 8.78 (d, 1H), 8.19 (m, 2H), 7.94 (s, 1H), 7.63 (m, 1H), 7.48 (d, 1H), 4.85-1.78 (m, 16H), 0.49-0.32 (m, 3H), −0.01 (m, 2H), MS (ESI) m/z: Calculated for C₂₆H₂₈F₃N₅O: 483.2; found: 484 (M+H)⁺.

### Example 18

### (3-(Cyclopropylmethyl)-1-(4-hydroxy-4-(6-methoxypyridin-3-yl)cyclohexyl)pyrrolidin-3-yl)(3-(trifluoromethyl)-7,8-dihydro-1,6-naphthyridin-6(5H)-yl)methanone

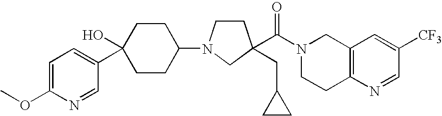

The title compound was prepared according to the procedures described in Example 30 (15 mg, 8% yield). ¹H NMR (400 MHz, CD₃OD): δ 8.63 (s, 1H), 8.15 (s, 1H), 7.96 (s, 1H), 7.65 (d, 1H), 6.74 (d, 1H), 4.73 (s, 2H), 3.88-3.60 (m, 6H), 3.24-3.04 (m, 6H), 2.42-1.56 (m, 12H), 0.59-0.37 (m, 3H), −0.04-0.23 (m, 2H); MS (ESI) m/z: Calculated for C₃₀H₃₇F₃N₄O₃: 558.3, found: 559 (M+H)⁺.

### Example 19

### 6-((3-(Cyclobutylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one

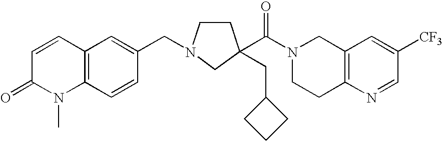

The title compound was prepared according to the general procedures described in Scheme 5 (7.1 mg TFA-salt, 4% yield): ¹H NMR (400 MHz, MeOH-d₄): δ 8.71 (s, 1H), 8.05 (m, 1H), 7.95 (d, 1H), 7.81 (d, 1H), 7.76 (s, 1H), 7.44 (d, 1H), 6.75 (d, 2H), 4.56 (m, 4H), 3.98 (m, 2H), 3.77 (s, 3H), 3.62-3.35 (m, 3H), 3.12 (m, 2H), 2.65 (s, 1H), 2.48 (m, 1H), 2.16 (m, 1H), 1.93 (m, 2H), 1.82-1.5 (m, 5H), 1.3-0.7 (m, 1H); MS (ESI) m/z: Calculated for C₃₀H₃₃F₃N₄O₂: 538.3; found 539.4 (M+H)⁺.

### Example 20

### 6-((3-Isopentyl-3-(3-(trifluoromethyl)-5,6,7,8-tetrahydro-1,6-naphthyridine-6-carbonyl)pyrrolidin-1-yl)methyl)-1-methylquinolin-2(1H)-one

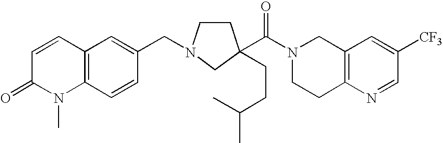

The title compound was prepared according to the general procedures described in Scheme 5 (9.0 mg TFA-salt, 2.4% yield): ¹H NMR (400 MHz, MeOH-d₄): δ 8.71 (s, 1H), 8.07 (m, 1H), 7.95 (d, 1H), 7.82 (d, 1H), 7.77 (s, 1H), 7.44 (d, 1H), 6.75 (dd, 2H), 4.55 (m, 4H), 3.98-3.39 (m, 4H), 3.78 (s, 3H), 3.10 (m, 4H), 2.63-1.20 (m, 3H), 1.10-0.50 (m, 9H); MS (ESI) m/z: Calculated for C₃₀H₃₅F₃N₄O₂: 540.3; found 541.4 (M+H)⁺.

### Example 21

### 6-((3-Benzyl-3-(3-(trifluoromethyl)-5,6,7,8-tetrahydro-1,6-naphthyridine-6-carbonyl)pyrrolidin-1-yl)methyl)-1-methylquinolin-2(1H)-one

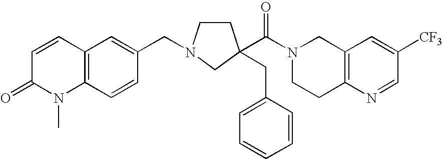

The title compound was prepared according to the general procedures described in Scheme 5 (17.0 mg TFA-salt, 3% yield): ¹H NMR (400 MHz, DMSO-d₆): δ 8.81 (s, 1H), 8.17 (m, 1H), 7.94 (dd, 1H), 7.79 (m, 1H), 7.77 (s, 1H), 7.38 (m, 1H), 7.10 (m, 5H), 6.69 (d, 1H), 4.85 (m, 2H), 4.53 (m, 2H), 3.91 (m, 3H), 3.37 (s, 3H), 3.12 (m, 6H), 2.97 (m, 1H), 2.52 (m, 1H), 2.3 (m, 1H); MS (ESI) m/z: Calculated for C₃₂H₃₁F₃N₄O₂: 560.2; found 561.3 (M+H)⁺.

### Example 22

### 6-((3-(Cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one

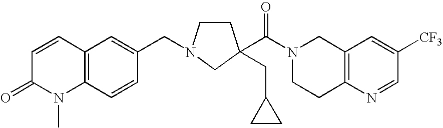

The title compound was prepared according to the general procedures described in Scheme 5 (21.0 mg TFA-salt, 15% yield): ¹H NMR (400 MHz, DMSO-d₆): δ 8.82 (s, 1H), 8.20 (m, 1H), 7.96 (m, 1H), 7.87 (s, 1H), 7.78 (m, 1H), 7.67 (d, 1H), 6.71 (d, 1H), 4.80 (m, 2H), 4.48 (s, 2H), 4.35-3.77 (m, 3H), 3.67 (s, 3H), 3.51-3.27 (m, 5H), 3.03 (m, 2H), 1.85 (m, 2H), 0.58-0.00 (m, 5H); MS (ESI) m/z: Calculated for C₂₉H₃₁F₃N₄O₂: 524.2; found 525.1 (M+H)⁺.

### Example 23

### 5-((3-(Cyclopropylmethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

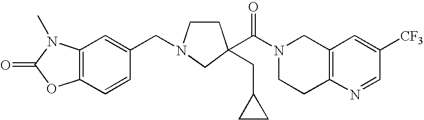

The title compound was prepared according to the general procedures described in Scheme 5 (5.2 mg TFA-salt, 1% yield): ¹H NMR (400 MHz, MeOH-d₄): δ 8.71 (s, 1H), 8.04 (s, 1H), 7.33 (m, 3H), 4.80 (m, 2H), 4.45 (m, 2H), 4.15-3.50 (m, 3H), 3.43 (s, 3H), 3.45-3.32 (m, 1H), 3.31-3.18 (m, 1H), 3.12 (m, 2H), 2.65 (s, 1H), 2.51 (m, 1H), 2.32-1.88 (m, 1H), 1.84 (m, 2H), 0.6-(−0.16) (m, 5H); MS (ESI) m/z: Calculated for C₂₇H₂₉F₃N₄O₃: 514.2; found 515.1 (M+H)⁺.

### Example 24

### 5-((3-Isobutyl-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

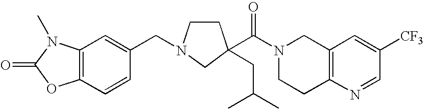

The title compound was prepared according to the general procedures described in Scheme 5 (21 mg TFA-salt, 19% yield): ¹H NMR (400 MHz, MeOH-d₄): δ 8.71 (s, 1H), 8.11-8.00 (m, 1H), 7.37-7.23 (m, 3H), 4.85 (m, 2H), 4.49-4.43 (m, 4H), 4.15-3.90 (m, 4H), 3.60-3.47 (m, 2H), 3.43 (s, 3H), 3.25-3.12 (m, 2H), 2.48-1.36 (m, 2H), 0.89-0.60 (m, 7H); MS (ESI) m/z: Calculated for C₂₇H₃₁F₃N₄O₃: 516.2; found 517.2 (M+H)⁺.

### Example 25

### 7-((3-(Methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one

The title compound was prepared via the intermediates shown below using the procedures described below.

### 1-tert-Butyl 3-methyl 3-(methoxymethyl)pyrrolidine-1,3-dicarboxylate

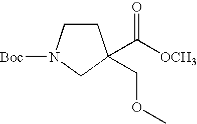

The title compound was prepared according to general procedure A described in connection with Scheme 2. 1-tert-Butyl 3-methylpyrrolidine-1,3-dicarboxylate (7.27 g, 31.7 mmol) was dissolved in 80 mL of THF under argon. 2M LDA in heptane/THF/Ethylbenzene (19 mL, 38 mmol) was added in 30 min. between −78° C. and −68° C. The reaction mixture was stirred at −78° C. for 45 min. Neat bromo(methoxy)methane (5.25 g, 41.2 mmol) was added over 11 min. at −78° C. The reaction was slowly warmed to RT and stirred for 20 h. The reaction mixture was cooled to −20° C. and quenched with 50 mL 10% NH₄Cl. The aqueous layer was extracted with 50 mL EtOAc. The organic layer was washed with 50 mL brine and then dried over Na₂SO₄.

The solvent was evaporated and the resulting residue was purified by 2 flash chromatographies on silica gel using Hexanes/EtOAc 10:1 to 2:1 and ⅓ to give 4.5 g (52% yield) of the desired product. MS (ESI) m/z: Calculated for C₁₃H₂₃NO₅: 273.2; found: 295 (M+Na)⁺.

### 1-(tert-Butoxycarbonyl)-3-(methoxymethyl)pyrrolidine-3-carboxylic acid, IIIc

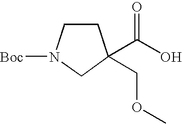

The title compound was prepared according to general procedure B described in connection with Scheme 2. 1-tert-Butyl 3-methyl 3-(methoxymethyl)pyrrolidine-1,3-dicarboxylate (4.5 g, 16.5 mmol) was dissolved in 30 mL of MeOH and a solution LiOH (0.79 g, 33 mmol) in 20 mL was added. The reaction mixture was microwaved at 130° C. for 25 min in 5 vials. The methanol was evaporated and the residue was acidified to pH 1-2 with KHSO₄ solid. The acid was extracted with EtOAc (3×50 mL). The combined organic fractions were washed with 1N KHSO₄ and with brine and then dried over Na₂SO₄. The solvent was evaporated to give 7.09 g of the desired acid (91% crude yield). ¹H NMR (400 MHz, CDCl₃): δ 10.70 (bs, 1H), 3.76 (d, 1H), 3.62-3.41 (m, 5H), 3.35 (s, 3H), 2.34-2.25 (m, 1H), 1.98-1.93 (m, 1H), 1.45 (s, 9H); MS (ESI) m/z: Calculated for C₁₂H₂₁NO₅: 259.1; found: 260 (M+1)⁺.

### tert-Butyl 3-(methoxymethyl)-3-(3-(trifluoromethyl)-5,6,7,8-tetrahydro-1,6-naphthyridine-6-carbonyl)pyrrolidine-1-carboxylate Vc

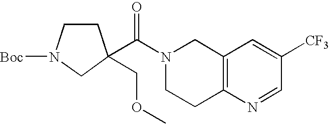

The title compound was prepared according to general procedure C described in connection with Scheme 2. To 1-(tert-butoxycarbonyl)-3-(methoxymethyl)pyrrolidine-3-carboxylic acid (3 g, 11.6 mmol) in dichloromethane (30 mL) was added 2M oxalyl chloride dichloromethane solution (17 mL, 34 mmole) and a few drops of DMF at room temperature. The mixture was stirred at RT for 2 hours and concentrated to dryness under the reduced pressure. 3-(Trifluoromethyl)-5,6,7,8-tetrahydro-1,6-naphthyridine dichloride (3.19 g, 11.6 mmole) in 30 dichloromethane and triethyl amine (3.1 g, 30 mmole) was added to the above residue at 0° C. The mixture was stirred at 0° C. for 1 h, diluted with dichloromethane (100 mL) and washed with sodium bicarbonate solution (2×50 mL) and water (3×100 mL), dried over sodium sulfate and purified by silica chromatography using 2.5% MeOH in CH₂Cl₂ as eluent to give product (1.7 g, 33% yield). ¹H NMR (400 MHz, CD₃OD): δ 8.70 (s, 1H), 7.68 (m, 1H), 4.80 (s, 2H), 4.10-2.95 (m, 13H), 2.40-2.15 (m, 2H), 1.48 (s, 9H); MS (ESI) m/z: Calculated for C₂₁H₂₈F₃N₃O₄: 443.2; found: 466 (M+Na)⁺.

### (3-(Methoxymethyl)pyrrolidin-3-yl)(3-(trifluoromethyl)-7,8-dihydro-1,6-naphthyridin-6(5H)-yl)methanone 2,2,2-trifluoroacetate Vic

The title compound was prepared via the following intermediate using the procedures described below.

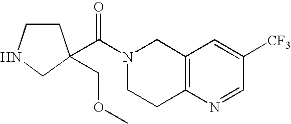

The title compound was prepared according to general procedure D described in connection with Scheme 2. tert-Butyl 3-(methoxymethyl)-3-(3-(trifluoromethyl)-5,6,7,8-tetrahydro-1,6-naphthyridine-6-carbonyl)pyrrolidine-1-carboxylate (1.7 g, 3.84 mmole) was dissolved in 50 mL CH₂Cl₂ and 16 mL TFA was added at RT. The reaction mixture was stirred for 17 h and the solvents evaporated. The residue was dissolved in CH₂Cl₂ and the organic layer was washed twice with 50 mL of saturated NaHCO₃ and then dried over Na₂SO₄. The solvent was evaporated to give product (1.3 g, quantitative yield) of the desired product. ¹H NMR (400 MHz, CDCl₃): δ 8.71 (s, 1H), 7.70 (s, 1H), 4.81 (s, 2H), 4.13-3.89 (m, 2H), 3.52-3.43 (m, 3H), 3.29 (s, 3H), 3.12-2.97 (m, 5H), 2.22-2.18 (m, 1H), 2.04-1.97 (m, 1H); MS (ESI) m/z: Calculated for C₁₆H₂₀F₃N₃O₂ (free base): 343.2; found: 344 (M+H)⁺.

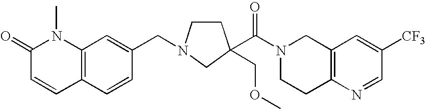

The title compound was prepared according to the general procedures described in Scheme 5. A mixture of (3-(methoxymethyl)pyrrolidin-3-yl)(3-(trifluoromethyl)-7,8-dihydro-1,6-naphthyridin-6(5H)-yl)methanone 2,2,2-trifluoroacetate (128.5 mg, 0.505 mmole), 7-(bromomethyl)-1-methylquinolin-2(1H)-one (289 mg, 0.505 mmole), K₂CO₃ (139 mg, 1 mmole) and DIEA (130 mg, 1 mmole) in DMF (3 mL) was microwaved at 100° C. for 30 min, diluted with ethyl acetate, washed with water, dried over sodium sulfate, filtered, concentrated and purified by reverse phase preparative HPLC to yield the desired final product with purity greater than 98% (32 mg TFA-salt, 8.5% yield). ¹H NMR (400 MHz, DMSO-d₆): δ 10.11 (s, 1H), 10.05 (s, 1H), 8.79 (s, 1H), 8.17 (s, 1H), 7.95 (d, 1H), 7.81 (d, 1H), 7.72 (d, 1H), 7.39 (d, 1H), 6.69 (d, 1H), 4.8-4.28 (m, 4H), 3.87-2.21 (m, 18H); MS (ESI) m/z: Calculated for C₂₇H₂₉F₃N₄O₃: 514.2; found: 515 (M+H)⁺.

### Examples 26 and 27

### (S)-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one and (R)-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one

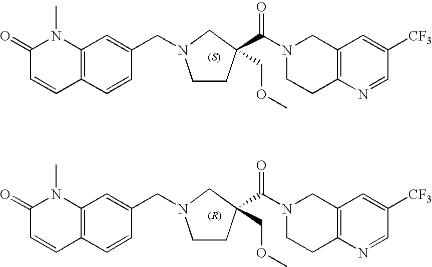

The racemic mixture (ca. 1:1 ratio) was separated into the two enantiomers by normal phase preparative HPLC using a chiral column, yielding enantiomer I (>95% ee; eluented at 6.38 min), and enantiomer II (>95% ee; eluented at 9.01 min).

### Example 28

### 5-((3-(Methoxymethyl)-3-(3-(trifluoromethyl)-5,6,7,8-tetrahydro-1,6-naphthyridine-6-carbonyl)pyrrolidin-1-yl)methyl)-3-methylbenzo[d]thiazol-2(3H)-one

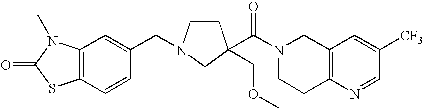

The title compound was prepared according to general procedures described in Scheme 5 using VIc (44.6 mg, 58.7% yield). ¹H NMR (400 MHz, CD₃Cl₃): δ 8.74 (s, 1H), 7.74 (s, 1H), 7.45 (d, 1H), 7.39 (s, 1H), 7.16 (d, 1H), 4.78 (m, 2H), 4.31 (dd, 2H), 3.90 (m, 4H), 3.75-3.60 (m, 4H), 3.45 (m, 3H), 3.24 (m, 1H), 3.15 (m, 2H), 2.67 (s, 3H), 2.46 (s, 1H); MS (ESI) m/z: Calculated for C₂₅H₂₇F₃N₄O₃S: 520.2; found: 521.1 (M+H)⁺.

### Example 29

### 5-(1-(3-(Methoxymethyl)-3-(3-(trifluoromethyl)-5,6,7,8-tetrahydro-1,6-naphthyridine-6-carbonyl)pyrrolidin-1-yl)ethyl)-1,3-dimethyl-1H-benzo[d]imidazol-2(3H)-one

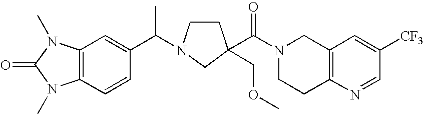

The title compound was prepared according to general procedures described in Scheme 6: A mixture of (3-(methoxymethyl)pyrrolidin-3-yl)(3-(trifluoromethyl)-7,8-dihydro-1,6-naphthyridin-6(5H)-yl)methanone VIc (50 mg, 0.146 mmol), 5-acetyl-1,3-dimethyl-1H-benzo[d]imidazol-2(3H)-one (30 mg, 0.146 mmol) and Ti(OⁱPr)₄ (51 mg, 0.182 mmol) in THF (2.5 mL) was irradiated in a Microwave instrument at 100° C. for 30 min (Personal Chemistry Emrys™ Optimizer microwave reactor). The reaction mixture was cooled and NaBH(OAc)₃ (93 mg, 0.438 mmol) was added. The mixture was stirred at room temperature overnight. The solvent was removed in vacuo. The residue was dissolved in 30 mL of ethyl acetate and washed with sat. NaHCO₃. The organic layer was washed with brine (×3), then dried over Na₂SO₄, filtered and concentrated under reduced pressure. The crude product was purified by reverse phase preparative HPLC to yield the desired final product as trifluoroacetic salt with purity greater than 95% (18.0 mg, 23.2% yield): ¹H NMR (300 MHz, CD₃Cl₃): δ 8.71 (s, 1H), 7.72 (s, 1H), 7.29 (bs, 1H), 7.01 (bs, 1H), 6.92 (s, 1H), 5.23 (m, 2H), 4.87 (m, 1H), 4.73 (m, 1H), 4.10-3.86 (m, 4H), 3.71 (m, 1H), 3.40 (m, 3H), 3.32-3.24 (m, 6H), 3.12 (m, 3H), 2.91 (bs, 1H), 2.66 (d, 1H), 2.42 (s, 1H), 2.30 (s, 1H), 1.81 (m, 2H); MS (ESI) m/z: Calculated for C₂₇H₃₂F₃N₅O₃: 531.3; found: 532.0 (M+H)⁺.

### Example 30 and 31

### 4-Hydroxy-4-(6-methoxypyridin-3-yl)cyclohexanone

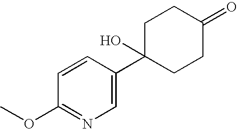

The title compound was prepared based on the procedures described in International Patent Application Publication No. WO 2004/050024, which is hereby incorporated by reference.

### (1-(4-Hydroxy-4-(6-methoxypyridin-3-yl)cyclohexyl)-3-(methoxymethyl)pyrrolidin-3-yl)(3-(trifluoromethyl)-7,8-dihydro-1,6-naphthyridin-6(5H)-yl)methanone

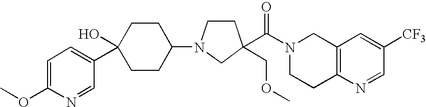

The title compound was prepared according to general procedures described in Scheme 2: A mixture of 4-hydroxy-4-(6-methoxypyridin-3-yl)cyclohexanone (71 mg, 0.32 mmol), (3-(methoxymethyl)pyrrolidin-3-yl)(3-(trifluoromethyl)-7,8-dihydro-1,6-naphthyridin-6(5H)-yl)methanone VIc (100 mg, 0.29 mmol) and acetic acid (20 μL, 0.34 mmol) in dichloroethane (3 mL) was stirred at room temperature for 0.5 h. Sodium triacetoxyborohydride (123 mg, 0.576 mmol) was added and the reaction mixture was stirred for 2 h at room temperature. After concentration of solvent under reduced pressure, the resulting residue was dissolved in ethyl acetate, then washed with NaHCO₃, water and brine. The organic extract was dried, filtered and concentrated. The crude product was obtained as a mixture of isomers which were further separated by reverse phase preparative HPLC to yield isomer A (eluted at 10.092 min) and isomer B (eluted at 11.269 min).

Isomer I (10.2 mg): ¹H NMR (300 MHz, MeOH-d₄): δ 8.71 (s, 1H), 8.30 (s, 1H), 8.04 (s, 1H), 7.98 (d, 1H), 6.93 (d, 1H), 4.87 (m, 2H), 3.94 (m, 5H), 3.90 (s, 1H) 3.66 (m, 3H), 3.36 (m, 1H), 3.10 (bs, 2H), 2.65 (s, 7H), 2.36-2.33 (m, 5H), 1.82 (m, 2H), 1.68 (m, 2H); MS (ESI) m/z: Calculated for C₂₈H₃₅F₃N₄O₄: 548.3; found: 549.2 (M+H)⁺.

Isomer II (4.4 mg): ¹H NMR (300 MHz, MeOH-d₄): δ 8.72 (s, 1H), 8.25 (d, 1H), 8.05 (s, 1H), 7.88 (dd, 1H), 6.86 (d, 1H), 4.87 (m, 2H), 4.00 (t, 2H), 3.92 (s, 4H), 3.70-3.65 (m, 3H), 3.36 (m, 1H), 3.12 (t, 2H), 2.65 (s, 7H), 2.37-2.30 (m, 1H), 2.16 (m, 1H), 2.06 (m, 3H), 1.94 (m, 4H); MS (ESI) m/z: Calculated for C₂₈H₃₅F₃N₄O₄: 548.3; found: 549.2 (M+H)⁺.

### Example 32

### 6-((3-(Methoxymethyl)-3-(3-(trifluoromethyl)-5,6,7,8-tetrahydro-1,6-naphthyridine-6-carbonyl)pyrrolidin-1-yl)methyl)-4-methyl-2H-benzo[b][1,4]oxazin-3(4H)-one

The title compound was prepared via the intermediate shown below using the procedures described below.

### 4-Methyl-3-oxo-3,4-dihydro-2H-benzo[b][1,4]oxazine-6-carbaldehyde

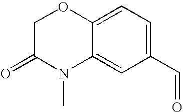

3-Oxo-3,4-dihydro-2H-benzo[b][1,4]oxazine-6-carbaldehyde (0.5 g, 2.82 mmol), methyl iodide (0.6 g, 4.23 mmol) and cesium carbonate (1.37 g, 4.23 mmol) were dissolved in dry dimethylformamide (2 mL). The reaction mixture was irradiated by microwave at 100° C. for 30 minutes. The solvent was removed and the residue was washed with water and extracted with dichloromethane. Purification by silica chromatography (ISCO) produced 4-methyl-3-oxo-3,4-dihydro-2H-benzo[b][1,4]oxazine-6-carbaldehyde (0.46 g, 85%). ¹H NMR (400 MHz, DMSO-d₆): δ9.95 (s, 1H), 7.62 (s, 1H), 7.59 (d, 1H), 7.20 (d, 1H), 4.80 (s, 2H), 3.34 (s, 3H); MS (ESI) m/z: Calculated for C₁₀H₉NO₃: 191.1; found: 192.1 (M+H)⁺.

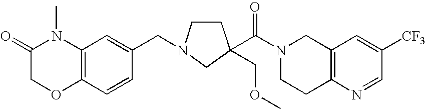

The title compound was prepared according to general procedures described in Scheme 5 using VIc (20 mg, 22% yield): ¹H NMR (300 MHz, DMSO-d₆): δ 8.80 (s, 1H), 8.10 (s, 1H), 7.30 (br s, 1H), 7.20-7.00 (m, 2H), 4.80 (s, 2H), 4.70 (s, 2H), 4.33 (s, 2H), 3.90 (s, 2H), 3.50-3.01 (m, 10H), 3.52 (s, 3H), 3.31 (s, 3H); MS (ESI) m/z: Calculated for C₂₆H₂₉F₃N₄O₄: 518.2; found: 519.3 (M+H)⁺.

### Example 33

### 6-(1-(3-(Methoxymethyl)-3-(3-(trifluoromethyl)-5,6,7,8-tetrahydro-1,6-naphthyridine-6-carbonyl)pyrrolidin-1-yl)ethyl)-4-methyl-2H-benzo[b][1,4]oxazin-3(4H)-one

The title compound was prepared via the intermediate shown below using the procedures described below.

### 6-Acetyl-4-methyl-2H-benzo[b][1,4]oxazin-3(4H)-one

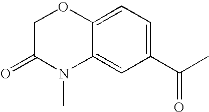

6-Acetyl-2H-benzo[b][1,4]oxazin-3(4H)-one (1 g, 5.23 mmol), methyl iodide (1.13 g, 7.84 mmol) and cesium carbonate (2.55 g, 7.84 mmol) were dissolved in DMF (2 mL). The reaction mixture was irradiated by microwave for 30 minutes at 100° C. The solvent was removed and extracted with dichloromethane. Purification by silica chromatography (ISCO) produced 6-acetyl-4-methyl-2H-benzo[b][1,4]oxazin-3(4H)-one (0.9 g, 84%). ¹H NMR (400 MHz, CDCl₃): δ 7.63 (s, 1H), 7.60 (d, 1H), 7.01 (d, 1H), 4.70 (s, 2H), 3.42 (s, 3H), 2.60 (s, 3H); MS (ESI) m/z: Calculated for C₁₁H₁₁NO₃: 205.1; found: 206.1 (M+H)⁺.

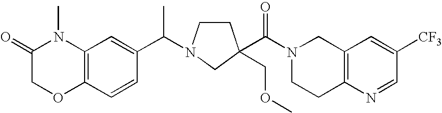

The title compound was prepared according to general procedures described in Scheme 6 using 1 equivalent of Ti(OiPr)₄ (5 mg, 4% yield): ¹H NMR (300 MHz, CDCl₃): δ 8.79 (s, 1H), 7.80 (s, 1H), 7.48-7.22 (m, 1H), 7.12-6.98 (m, 2H), 5.01-4.50 (m, 5H), 4.22-3.70 (m, 6H), 3.40-3.22 (m, 8H), 2.61-2.30 (m, 4H), 1.80 (d, 3H); MS (ESI) m/z: Calculated for C₂₇H₃₁F₃N₄O₄: 532.2; found: 533.2 (M+H)⁺.

### Example 34

### 5-(1-(3-(Methoxymethyl)-3-(3-(trifluoromethyl)-5,6,7,8-tetrahydro-1,6-naphthyridine-6-carbonyl)pyrrolidin-1-yl)ethyl)-3-methylbenzo[d]oxazol-2(3H)-one

The title compound was prepared via the intermediate shown below using the procedures described below.

### 5-Acetyl-3-methylbenzo[d]oxazol-2(3H)-one

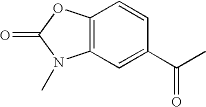

5-Acetylbenzo[d]oxazol-2(3H)-one (0.5 g, 2.82 mmol), methyl iodide (0.6 g, 4.23 mmol), and cesium carbonate (1.37 g, 4.23 mmol) were dissolved in DMF (2 mL). The reaction mixture was irradiated by microwave for 30 minutes at 100° C. The solvent was removed and extracted with dichloromethane. Purification by silica chromatography (ISCO) produced 5-acetyl-3-methylbenzo[d]oxazol-2(3H)-one (0.4 g, 74%). ¹H NMR (400 MHz, CDCl₃): δ 7.89 (d, 1H), 7.80 (s, 1H), 7.00 (d, 1H), 3.48 (s, 3H), 2.60 (s, 3H); MS (ESI) m/z: Calculated for C₁₀H₉NO₃: 191.1; found: 192.1 (M+H)⁺.

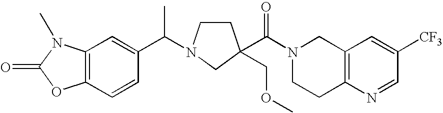

The title compound was prepared according to general procedures described in Scheme 6 using 1 equivalent of Ti(OiPr)₄ (9 mg, 7% yield): ¹H NMR (300 MHz, CDCl₃): δ 8.70 (s, 1H), 7.70 (s, 1H), 7.50-7.48 (m, 2H), 7.01 (m, 1H), 4.80-4.70 (m, 2H), 4.20-4.03 (m, 2H), 3.90-3.81 (m, 3H), 3.40 (s, 6H), 3.31-3.05 (m, 6H), 2.60-2.48 (m, 2H) 1.80 (d, 3H); MS (ESI) m/z: Calculated for C₂₆H₂₉F₃N₄O₄: 518.2; found: 519.2 (M+H)⁺.

### Example 35

### (1-(4-Cyclopropyl-4-hydroxycyclohexyl)-3-(methoxymethyl)pyrrolidin-3-yl)(3-trifluoromethyl)-7,8-dihydro-1,6-naphthyridin-6(5H)-yl)methanone

The title compound was prepared via the intermediates shown below using the procedures described below.

### 8-Cyclopropyl-1,4-dioxaspiro[4.5]decan-8-ol

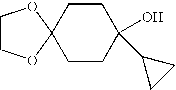

1,4-Dioxaspiro[4.5]decan-8-one (2 g, 12.80 mmol) was dissolved in dry tetrahydrofuran (20 mL). The reaction mixture was cooled to −78° C. Cyclopropylmagnesium bromide (51.2 mL, 25.61 mmol) was added dropwise over a period of 10 min. The reaction mixture was stirred for 4 h, quenched with sat. NH₄Cl and extracted with ethyl acetate. The solvent was evaporated and the crude product was used without further purification in the next step.

### 4-Cyclopropyl-4-hydroxycyclohexanone

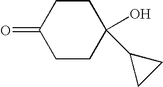

8-Cyclopropyl-1,4-dioxaspiro[4.5]decan-8-ol (1.8 g) was dissolved in acetone (30 mL). A solution of water (15 mL) and conc. HCl was added. The reaction mixture was stirred overnight. The reaction mixture was taken in ethyl acetate and washed with sat. NaHCO₃. Purification by silica chromatography produced 4-cyclopropyl-4-hydroxycyclohexanone (0.5 g, 27%). ¹H NMR (400 MHz, CDCl₃): δ 2.80-2.69 (m, 2H), 2.50-2.42 (m, 2H), 1.99-1.78 (m, 4H), 1.01-0.99 (m, 1H), 0.50-0.47 (m, 4H); MS (ESI) m/z: Calculated for C₉H₁₄O₂: 154.1; found: 155.1 (M+H)⁺.

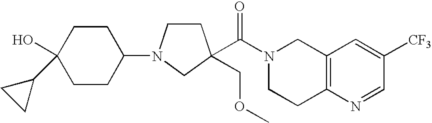

The title compound was prepared according to general procedures described in Scheme 8 (12 mg, 6% yield): ¹H NMR (300 MHz, CDCl₃): δ 11.50 (br s, 1H), 8.80 (s, 1H), 7.82 (s, 1H), 4.90-4.68 (m, 4H), 3.99-3.03 (m, 8H), 3.35 (s, 3H), 2.77-2.72 (m, 1H), 2.60-1.50 (m, 10H), 0.05-0.01 (m, 5H); MS (ESI) m/z: Calculated for C₂₅H₃₄F₃N₃O₃: 481.3; found: 482.2 (M+H)⁺.

### Example 36

### 6-Chloro-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one

The title compound was prepared via the intermediates shown below using the procedures described below.

### N-(4-Chloro-3-methylphenyl)cinnamamide

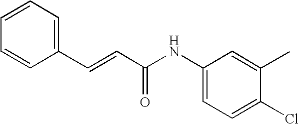

To 4-chloro-3-methylaniline (3.33 g, 19.98 mmole) in dichloromethane (100 mL) and pyridine (20 mL) was added cinnamoyl chloride (2.83 g, 19.98 mmole) at 0° C. The mixture was stirred at room temperature for 2 h. Solvent and pyridine were removed by rotary evaporator under reduced pressure. The residue was diluted with ethyl acetate and washed 1N HCl (2×100 mL), water (2×100 mL), sodium bicarbonate solution (2×100 mL) and dried over sodium sulfate. After removing solvent a white solid was recovered (5.38 g, 99% yield). The product was used for the next step without further purification. ¹H NMR (400 MHz, DMSO-d₆): δ 7.76-7.24 (m, 10H), 6.58 (d, 1H), 2.55 (s, 3H); MS (ESI) m/z: Calculated for C₁₆H₁₄ClNO: 271.1; found: 272 (M+H)⁺.

### 6-Chloro-7-methylquinolin-2(1H)-one

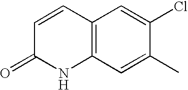

A mixture of N-(4-chloro-3-methylphenyl)cinnamamide (2.75 g, 10.15 mmole) and aluminum chloride (1.35 g, 10.15 mmole) was heated at 100° C. for 2 h. Ice was added to the reaction mixture. The mixture was extracted with dichloromethane, dried over sodium sulfate and purified by silica gel flash chromatography (eluted with 4% methanol in dichloromethane) to give a mixture of isomers (1:1) (600 mg), 6-chloro-7-methylquinolin-2(1H)-one and 6-chloro-5-methylquinolin-2(1H)-one. The mixture was used for the next step without further purification. MS (ESI) m/z: Calculated for C₁₀H₈ClNO: 193.0; found: 194 (M+H)⁺.

### 6-Chloro-1,7-dimethylquinolin-2(1H)-one

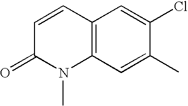

A mixture of 6-chloro-7-methylquinolin-2(1H)-one, 6-chloro-5-methylquinolin-2(1H)-one, Cs₂CO₃, and iodomethane in DMF was heated in a microwave reactor at 100° C. for 30 min. The mixture was diluted with dichloromethane, washed with water and dried over sodium sulfate. After removing the solvent the crude product (600 mg) was used in the next step without purification. MS (ESI) m/z: Calculated for C₁₁H₁₀ClNO: 207.1; found: 208 (M+H)⁺.

### 7-(Bromomethyl)-6-chloro-1-methylquinolin-2(1H)-one

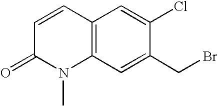

Crude 6-Chloro-1,7-dimethylquinolin-2(1H)-one (600 mg) dissolved in carbon-tetrachloride (30 ml) was added NBS (515.9 mg, 2.9 mmole) and AIBN (23.8 mg, 0.145 mmole). The mixture was heated under reflux for 3 h, diluted with dichloromethane, washed by sodium bicarbonate solution and water, dried over sodium sulfate, purified by silica chromatography (eluted with dichloromethane) to give a mixture of two isomers (550 mg). This mixture was used for the next step with out further purification. MS (ESI) m/z: Calculated for C₁₁H₉BrClNO: 285; found: 286 (M+H)⁺.

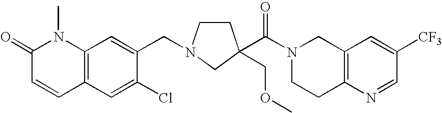

The title compound was prepared according to general procedures described in Scheme 5 using VIc (5 mg, 1% yield). ¹H NMR (400 MHz, DMSO-d₆): δ 10.12 (bs, 1H), 10.05 (bs, 1H), 8.79 (s, 1H), 8.42 (d, 1H), 8.17 (s, 1H), 7.82 (m, 1H), 7.42 (s, 1H), 6.68 (d, 1H), 4.80-2.21 (m, 20H); MS (ESI) m/z: Calculated for C₂₇H₂₈ClF₃N₄O₃: 548.2; found: 549 (M+H)⁺.

### Example 37

### 6-Chloro-5-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

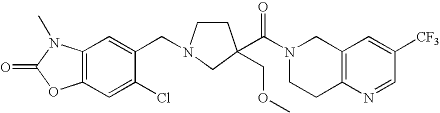

The title compound was prepared according to general procedures described in Scheme 5 using VIc (10 mg, 6% yield). ¹H NMR (400 MHz, CD₃OD): δ 8.61 (s, 1H), 7.93 (s, 1H), 7.45 (s, 1H), 7.35 (s, 1H), 4.53 (s, 2H), 3.88-2.47 (m, 18H), 2.49-2.47 (m, 2H); MS (ESI) m/z: Calculated for C₂₅H₂₆ClF₃N₄O₄: 538.2; found: 539 (M+H)⁺.

### Example 38

### 5-((3-(Methoxymethyl)-3-(3-(trifluoromethyl)-5,6,7,8-tetrahydro-1,6-naphthyridine-6-carbonyl)pyrrolidin-1-yl)methyl)-1,3-dimethyl-1H-benzo[d]imidazol-2(3H)-one

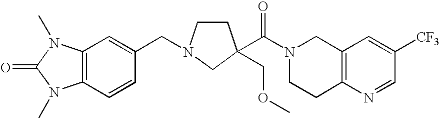

The title compound was prepared according to general procedures described in Scheme 5 (15 mg, 8% yield). ¹H NMR (400 MHz, CD₃OD): δ 8.61 (s, 1H), 7.93 (s, 1H), 7.20-7.15 (m, 3H), 4.56 (s, 2H), 3.86-2.24 (m, 23H); MS (ESI) m/z: Calculated for C₂₆H₃₀F₃N₅O₃: 517.2, found: 518 (M+H)⁺.

### Example 39

### 5-((3-(Methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

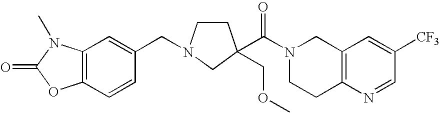

The title compound was prepared according to the general procedures described in Scheme 5 using VIc (15.3 mg TFA-salt, 17% yield): ¹H NMR (400 MHz, MeOH-d₄): δ 8.70 (s, 1H), 8.03 (s, 1H), 7.33 (m, 3H), 4.84 (m, 2H), 4.45 (m, 4H), 3.95 (m, 4H), 3.78-3.52 (m, 4H), 3.43 (s, 3H), 3.40-3.20 (m, 1H), 3.09 (m, 2H), 2.65 (s, 2H); MS (ESI) m/z: Calculated for C₂₅H₂₇F₃N₄O₄: 504.2; found 505.1 (M+H)⁺.

### Example 40 and 41

### (R)-6-((3-(Methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one and (S)-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

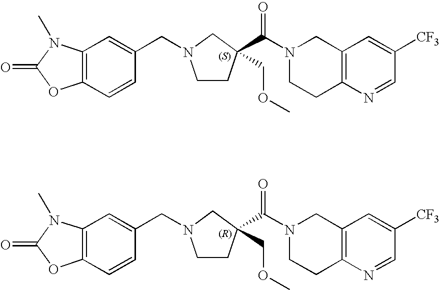

The racemic mixture (ca. 1:1 ratio) was separated into the two enantiomers by normal phase preparative HPLC using a chiral column, yielding enantiomer I (eluted at 6.98 min) and enantiomer II (eluted at 14.47 min).

Enantiamer I>99% ee; ¹H NMR (300 MHz, CDCl₃): δ 8.69 (s, 1H), 7.65 (s, 1H), 7.12 (m, 3H), 7.06 (d, 1H), 6.98 (s, 1H), 4.82 (m, 2H), 4.04-3.99 (m, 2H), 3.98-3.62 (m, 4H), 3.23 (m, 3H), 3.08-2.96 (m, 3H), 2.78-2.74 (m, 2H), 2.51 (m, 1H), 2.25 (m, 1H), 2.06-2.00 (m, 1H), 1.63 (m, 1H); MS (ESI) m/z: Calculated for C₂₅H₂₇F₃N₄O₄: 504.54; found 505.1 (M+H)⁺.

Enantiamer II >98.6% ee; ¹H NMR (300 MHz, CDCl₃): δ 8.69 (s, 1H), 7.61 (s, 1H), 7.12 (d, 1H), 7.06 (d, 1H), 6.98 (s, 1H), 4.83 (m, 2H), 4.06-3.74 (m, 2H), 3.64-3.57 (m, 4H), 3.23 (m, 3H), 3.08 (m, 2H), 3.06 (m, 2H), 2.97 (m, 1H), 2.78-2.71 (m, 2H), 2.52 (m, 1H) 2.30 (m, 1H), 2.06 (m, 1H), 1.62 (m, 1H); MS (ESI) m/z: Calculated for C₂₅H₂₇F₃N₄O₄: 504.54; found 505.1 (M+H)⁺.

### Example 42

### 3-Ethyl-6-((3-(methoxymethyl)-3-(3-(trifluoromethyl)-5,6,7,8-tetrahydro-1,6-naphthyridine-6-carbonyl)pyrrolidin-1-yl)methyl)benzo[d]oxazol-2(3H)-one

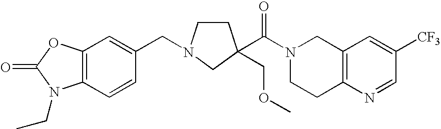

The title compound was prepared according to the general procedures described in Scheme 5 using VIc (9.0 mg TFA-salt, 3% yield): ¹H NMR (400 MHz, MeOH-d₄): δ 8.61 (s, 1H), 7.94 (s, 1H), 7.25 (m, 3H), 4.75 (m, 2H), 4.35 (m, 2H), 3.86 (m, 4H), 3.56 (m, 4H), 3.43 (s, 3H), 3.15 (m, 2H), 2.99 (m, 2H), 2.30 (m, 2H), 1.28 (t, 3H); ¹H NMR (400 MHz, DMSO-d₆): δ 8.79 (s, 1H), 8.16 (s, 1H), 7.45 (m, 2H), 7.28 (m, 1H), 4.79 (m, 2H), 4.42 (m, 2H), 3.85 (m, 5H), 3.75-3.35 (m, 4H), 3.34-2.92 (m, 7H), 2.30-2.05 (m, 1H), 1.28 (t, 3H); MS (ESI) m/z: Calculated for C₂₆H₂₉F₃N₄O₄: 518.2; found 519.2 (M+H)⁺.

### Example 43

### 5-Bromo-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

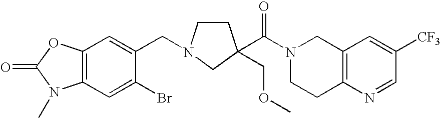

The title compound was prepared according to the general procedures described in Scheme 5 using VIc (45.0 mg TFA-salt, 3% yield): ¹H NMR (300 MHz, MeOH-d₄): δ 8.61 (s, 1H), 7.94 (s, 1H), 7.61 (s, 1H), 7.36 (s, 1H), 4.76 (m, 2H), 4.54 (m, 2H), 3.86 (m, 2H), 3.60 (m, 3H), 3.45 (m, 3H), 3.33 (s, 3H), 3.25 (m, 3H), 3.01 (m, 2H), 2.54 (m, 2H); ¹H NMR (300 MHz, DMSO-d₆): δ 8.79 (s, 1H), 8.17 (s, 1H), 7.86 (s, 1H), 7.58 (s, 1H), 4.81 (m, 2H), 4.70-4.30 (m, 3H), 3.88 (m, 3H), 3.80-3.50 (m, 4H), 3.34 (m, 3H), 3.30-3.10 (m, 3H), 3.01 (m, 2H), 2.60-2.20 (m, 2H); MS (ESI) m/z: Calculated for C₂₅H₂₆BrF₃N₄O₄: 582.1; found 583.2 (M+H)⁺.

### Example 44

### 6-((3-(Methoxymethyl)-3-(3-(trifluoromethyl)-5,6,7,8-tetrahydro-1,6-naphthyridine-6-carbonyl)pyrrolidin-1-yl)methyl)-3,5-dimethylbenzo[d]oxazol-2(3H)-one

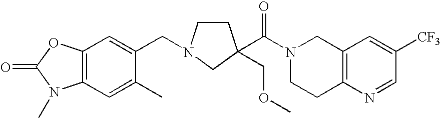

5-Bromo-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one (30 mg, 0.04 mmol) was dissolved in THF under nitrogen in a microwave tube. Methylzinc chloride (0.06 mL of a 2M solution, 0.12 mmol) and Pd(PtBu₃)₂(1 mg, 0.002 mmol) were added. The reaction mixture was purged with nitrogen for 5 min and subsequently heated at 100° C. for 30 min under microwave irradiation (Personal Chemistry Emrys™ Optimizer). Upon completion of the reaction the mixture was diluted with ethyl acetate, washed with 1N HCl aqueous solution, brine and filtered through celite. The filtrate was dried over Na₂SO₄ and concentrated. The residue was purified by preparative HPLC to give the pure product (1 mg TFA-salt, 19% yield): ¹H NMR (400 MHz, MeOH-d₄): δ 8.72 (s, 1H), 8.05 (s, 1H), 7.28 (m, 2H), 4.52 (m, 4H), 3.97 (m, 4H), 3.70 (m, 4H), 3.53-3.50 (m, 5H), 3.20-3.10 (m, 3H), 2.70-2.50 (m, 2H) 2.51 (s, 3H); MS (ESI) m/z: Calculated for C₂₆H₂₉F₃N₄O₄: 518.2; found 519.2 (M+H)⁺.

### Example 45

### 5-((3-(Ethoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

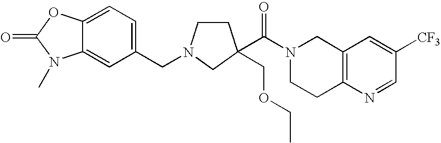

The title compound was prepared according to the general procedures described in Scheme 5 (110.0 mg): ¹H NMR (400 MHz, CDCl₃): δ 8.69 (s, 1H), 7.64 (s, 1H), 7.58 (s, 1H), 7.13-6.94 (m, 3H), 4.60-4.03 (m, 2H), 3.83-3.78 (m, 1H), 3.62 (s, 3H), 3.58-3.55 (m, 2H), 3.40-3.26 (m, 5H), 3.10-3.03 (m, 2H), 2.96-2.93 (m, 1H), 2.78-2.73 (m, 2H), 2.55-2.46 (m, 1H), 2.30-2.23 (m, 1H), 2.09-2.02 (m, 1H), 1.02-0.98 (m, 3H); MS (ESI) m/z: Calculated for C₂₆H₂₉F₃N₄O₄: 518.53; found 519.2 (M+H)⁺.

### Example 46

### Hydrochloride salt of 5-((3-(methoxymethyl)-3-(7-(trifluoromethyl)-1,2,3,4-tetrahydroisoquinoline-2-carbonyl)pyrrolidin-1-yl)methyl)-3-methylbenzo[d]oxazol-2(3H)-one

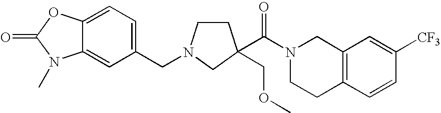

The title compound was prepared according to the general procedures described in Scheme 5 using VIc (100.0 mg): ¹H NMR (400 MHz, DMSO-d₆): δ 7.69-7.61 (m, 2H), 7.55-7.51 (m, 2H), 7.00-6.92 (m, 2H), 4.80-4.63 (m, 2H), 4.40-4.32 (m, 2H), 3.80-3.60 (m, 4H), 3.50-3.40 (m, 2H), 3.38 (s, 3H), 3.36 (s, 3H), 3.20-3.01 (m, 2H), 2.95-2.88 (m, 1H), 2.77-2.74 (m, 1H), 2.44-2.41 (m, 1H), 2.30-2.20 (m, 1H); MS (ESI) m/z: Calculated for C₂₆H₂₈F₃N₃O₄: 503.51; found 504.2 (M+H)⁺.

### Example 47

### Hydrochloride salt of 5-((3-isopropyl-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

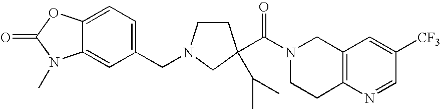

The title compound was prepared according to the general procedures described in Scheme 5 (48.5 mg): ¹H NMR (400 MHz, DMSO-d₆): δ 8.76 (s, 1H), 8.16 (s, 1H), 7.47-7.32 (m, 3H), 4.82-4.79 (m, 2H), 4.45-4.19 (m, 2H), 3.83-3.79 (m, 2H), 3.40-3.35 (m, 2H), 3.54 (s, 3H), 3.00-2.85 (m, 4H), 2.59-2.56 (m, 2H), 2.22-2.03 (m, 1H), 0.98 (d, 6H); MS (ESI) m/z: Calculated for C₂₆H₂₉F₃N₄O₃: 502.53; found 503.1 (M+H)⁺.

### Example 48

### Hydrochloride salt of 2-(1-((3-methyl-2-oxo-2,3-dihydrobenzo[d]oxazol-5-yl)methyl)-3-(3-(trifluoromethyl)-5,6,7,8-tetrahydro-1,6-naphthyridine-6-carbonyl)pyrrolidin-3-yl)acetonitrile

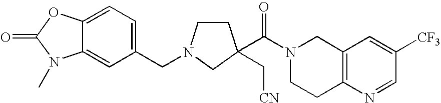

The title compound was prepared according to the general procedures described in Scheme 5 (480.0 mg): ¹H NMR (400 MHz, DMSO-d₆): δ 8.80 (s, 1H), 8.20 (s, 1H), 7.61-7.61-7.30 (m, 3H), 4.81-4.79 (m, 2H), 4.50-4.39 (m, 2H), 3.90-3.80 (m, 2H), 3.60-3.44 (m, 2H), 3.39 (s, 3H), 3.20-3.00 (m, 2H), 2.85-2.79 (m, 3H), 2.45-2.25 (m, 1H), 2.35-2.20 (m, 1H), 2.30-2.20 (m, 1H); MS (ESI) m/z: Calculated for C₂₅H₂₄F₃N₅O₃: 499.48; found 500.3 (M+H)⁺.

### Example 49

### 5-((3-(Hydroxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one

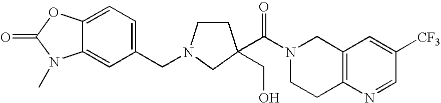

To a stirred solution of compound from Example 39 (770 mg, 1.52 mmol) in 14 mL of DCM at −78° C. was added a solution of BBr₃ (4.58 mmol) in DCM. After 1 hour, the reaction mixture was warned up to −25° C. and the reaction was monitored by TLC. After completion, the mixture was quenched with sat. NaHCO₃. The organic layer was separated and the aqueous layer was extracted with ethyl acetate (3×100 mL). The combinated organic layer were washed with brine, dried over MgSO₄ and concentrated to gave the crude product, which was purified by a flash column chromatography give 172 mg of the title compound (0.351 mmol, 23%): ¹H NMR (400 MHz, CD₃OD): δ 8.63 (s, 1H), 7.89 (s, 1H), 7.20-7.15 (m, 3H), 4.83-4.79 (m, 2H), 4.15-3.85 (m, 2H), 3.81-3.60 (m, 2H), 3.40 (s, 2H), 3.35 (s, 3H), 3.30-3.00 (m, 3H), 2.80-2.60 (m, 3H), 2.32-2.20 (m, 1H), 2.10-1.99 (m, 1H); MS (ESI) m/z: Calculated for C₂₄H₂₅F₃N₄O₄: 490.47; found 491.1 (M+H)⁺.

### Example 50 and 51

### (3-(Methoxymethyl)-1-(4-(6-methoxypyridin-3-yl)cyclohexyl)pyrrolidin-3-yl)(3-(trifluoromethyl)-7,8-dihydro-1,6-naphthyridin-6(5H)-yl)methanone

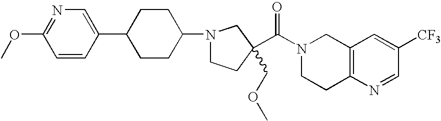

The title compound was prepared according to the procedures described in Scheme 8 (Step 2) using compounds from Examples 52 and 53: The crude product was obtained as a mixture of isomers which were further separated by reverse phase preparative HPLC to yield isomer I and isomer II.

Isomer I (1.3 mg): ¹H NMR (300 MHz, CDCl₃): δ 8.70 (s, 1H), 8.00 (s, 1H), 7.67 (s, 1H), 7.42 (m, 1H), 6.68 (d, 1H), 4.89 (m, 2H), 3.91 (m, 2H), 3.50 (m, 3H), 3.26 (s, 3H), 3.11 (s, 3H), 2.75 (m, 3H), 2.258 (m, 8H), 1.50 (m, 6H); MS (ESI) m/z: Calculated for C₂₈H₃₅F₃N₄O₃: 532.60; found: 533.4 (M+H)⁺.

Isomer II (16.1 mg): ¹H NMR (300 MHz, CDCl₃): δ 8.70 (s, 1H), 8.00 (s, 1H), 7.67 (s, 1H), 7.42 (m, 1H), 6.68 (d, 1H), 4.89 (m, 2H), 3.91 (m, 2H), 3.50 (m, 3H), 3.26 (s, 3H), 3.11 (s, 3H), 2.75 (m, 3H), 2.258 (m, 8H), 1.50 (m, 6H); MS (ESI) m/z: Calculated for C₂₈H₃₅F₃N₄O₃: 532.60; found: 533.2 (M+H)⁺.

### Example 52 and 53

### (1-(4-Hydroxy-4-(pyrimidin-5-yl)cyclohexyl)-3-(methoxymethyl)pyrrolidin-3-yl)(3-(trifluoromethyl)-7,8-dihydro-1,6-naphthyridin-6(5H)-yl)methanone

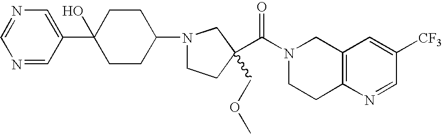

The title compound was prepared according to the procedure in Examples 30 and 31 (Scheme 8). The crude product was obtained as a mixture of isomers which were further separated by reverse phase preparative HPLC to yield isomer I and isomer II.

Isomer I: MS (ESI) m/z: Calculated for C₂₆H₃₂F₃N₅O₃: 519.56; found: 520 (M+H)⁺.

Isomer II: MS (ESI) m/z: Calculated for C₂₆H₃₂F₃N₅O₃: 519.56; found: 520 (M+H)⁺.

### Example 54 and 55

### (3-(Methoxymethyl)-1-(4-(pyrimidin-5-yl)cyclohexyl)pyrrolidin-3-yl)(3-(trifluoromethyl)-7,8-dihydro-1,6-naphthyridin-6(5H)-yl)methanone

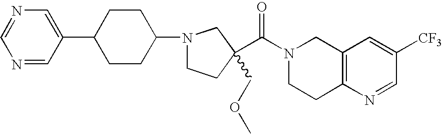

The title compound was prepared according to the procedures described in Scheme 8 (Step 2) using compounds from Examples 52 and 53. The crude product was obtained as a mixture of isomers which were further separated by reverse phase preparative HPLC to yield isomer I and isomer II.

Isomer I (33.8 mg): ¹H NMR (400 MHz, CD₃OD): δ 8.89 (s, 1H), 8.74-8.70 (m, 2H), 8.69 (s, 1H), 8.10 (s, 1H), 4.85-4.82 (m, 2H), 4.10-3.90 (m, 2H), 3.70-3.57 (m, 2H), 3.40 (s, 3H), 3.00-2.70 (m, 4H), 2.55-2.12 (m, 3H), 2.10-1.90 (m, 5H), 1.80-1.65 (m, 4H), 1.40-1.25 (m, 2H); MS (ESI) m/z: Calculated for C₂₆H₃₂F₃N₅O₂: 503.55; found: 504 (M+H)⁺.

Isomer II: ¹H NMR (400 MHz, CD₃OD): δ 8.99 (s, 1H), 8.71 (s, 2H), 8.68 (s, 1H), 8.01 (s, 1H), 4.90 (m, 2H), 4.05 (m, 1H), 3.94 (m, 1H), 3.60 (m, 2H), 3.30-1.35 (m, 23H); MS (ESI) m/z: Calculated for C₂₆H₃₂F₃N₅O₂: 503.56; found: 504 (M+H)⁺.

### Example 56 and 57

### (1-(4-(4-Fluorophenyl)cyclohexyl)-3-(methoxymethyl)pyrrolidin-3-yl)(3-(trifluoromethyl)-7,8-dihydro-1,6-naphthyridin-6(5H)-yl)methanone

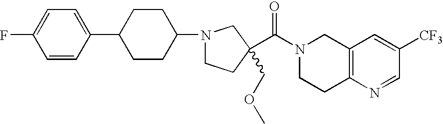

The title compounds were prepared according to the general procedures described in Scheme 8. The crude product was obtained as a mixture of isomers which were further separated by reverse phase preparative HPLC to yield isomer I and isomer II.

Isomer I (60 mg): ¹H NMR (400 MHz, CD₃OD): δ 8.95 (d, 1H), 8.43 (d, 1H), 7.41 (m, 2H), 7.01 (m, 2H), 4.90 (m, 2H), 4.65 (m, 1H), 4.05 (m, 3H), 3.72 (m, 4H), 3.45-1.70 (m, 17H); MS (ESI) m/z: Calculated for C₂₈H₃₃F₄N₃O₂: 519.57; found: 520 (M+H)⁺.

Isomer II: (30 mg): ¹H NMR (400 MHz, CD₃OD): δ 8.83 (s, 1H), 8.24 (s, 1H), 7.23 (m, 2H), 7.01 (m, 2H), 4.90 (m, 2H), 4.61 (m, 1H), 4.00 (m, 3H), 3.72 (m, 4H), 3.45-1.60 (m, 17H); MS (ESI) m/z: Calculated for C₂₈H₃₃F₄N₃O₂: 519.57; found: 520 (M+H)⁺.

### Example 58

### 6-((4-Isobutyl-4-(3-(trifluoromethyl)-5,6,7,8-tetrahydro-1,6-naphthyridine-6-carbonyl)piperidin-1-yl)methyl)-3-methylbenzo[d]thiazol-2(3H)-one di-TFA salt

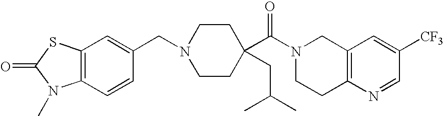

The title compound was prepared according to the general procedures described in Scheme 4 (15 mg, 4.5% yield): ¹H NMR (400 MHz, DMSO-d6): δ 9.56 (bs, 2H), 8.77 (s, 1H), 8.22 (s, 1H), 7.79-7.73 (m, 1H), 7.43 (s, 1H), 7.30 (d, 1H), 5.00-4.84 (m, 2H), 4.31-4.30 (m, 2H), 4.02-3.90 (m, 2H), 3.48 (s, 3H), 3.34-2.80 (m, 6H), 2.20-1.31 (m, 7H), 0.90-0.80 (m, 6H); MS (ESI) m/z: Calculated for C₂₈H₃₃F₃N₄O₂S: 546.65; found 547 (M+H)⁺.

### Example 59

### 6-((4-Isobutyl-4-(3-(trifluoromethyl)-5,6,7,8-tetrahydro-1,6-naphthyridine-6-carbonyl)piperidin-1-yl)methyl)-3-methylbenzo[d]oxazol-2(3H)-one

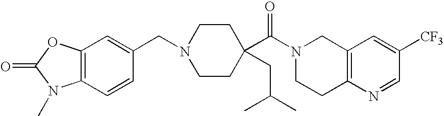

The title compound was prepared according to the general procedures described in Scheme 4 (48 mg): ¹H NMR (400 MHz, DMSO-d6): δ 8.62 (s, 1H), 7.97 (s, 1H), 7.24-7.13 (m, 3H), 4.80 (m, 2H), 4.20 (s, 2H), 3.94 (s, 2H), 3.32 (m, 5H), 3.10 (m, 4H), 2.56 (d, 2H), 1.59 (m, 4H), 0.713 (m, 6H); MS (ESI) m/z: Calculated for C₂₈H₃₃F₃N₄O₃: 530.58; found 531.2 (M+H)⁺.

### Example 60

The biological activity of the compounds described herein can be evaluated using assays known in the art, such as the AequoScreen™ assay.

### General Procedures for AequoScreen™ Assay:

AequoScreen™ CCR2b (FAST-060A) cells grown to mid-log phase in culture media without antibiotics are detached with PBS-EDTA, centrifuged and resuspended in assay buffer (DMEM/HAM's F12 with HEPES, without phenol red+0.1% BSA protease free) at a concentration of 1×10⁶ cells/mL. Cells are incubated at room temperature for at least 4 hours with coelenterazine h. Dose response curves are performed before testing. The reference agonist is MCP-1.

For agonist testing, 50 μL of cell suspension is mixed with 50 μL of test compound in a 96-well plate. The resulting emission of light is recorded using a Hamamatsu Functional Drug Screening System 6000 (FDSS 6000).

Following an incubation of 15 minutes after the first injection, 100 μL of the resulting cell suspension containing the test compound is mixed with 100 μL of the reference agonist in the 96 well test plate. The resulting emission of light is recorded using the same luminometer as for agonist testing.

To standardize the emission of recorded light (determination of the “100% signal”) across plates and across different experiments, some of the wells contained 100 μM digitonin, a saturating concentration of ATP (20 μM) and a concentration of reference agonist equivalent to the EC₅₀ obtained during test validation.

Agonist activity of a test compound can be expressed as a percentage of the activity of the reference agonist at its EC₁₀₀ concentration. Antagonist activity of a test compound can be expressed as a percentage of the inhibition of reference agonist activity at its EC₈₀ concentration.

### Results:

Biological activity data for several compounds collected using the above method is provided in Table 2.

TABLE 2

| | | IC₅₀ (μM) |
|---|---|---|
| Example No. | Structure | Ca⁺⁺flux |
| 1 | 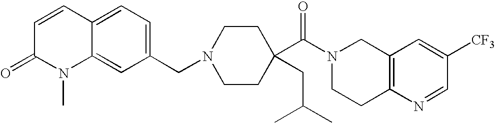 | ++ |
| 2 | 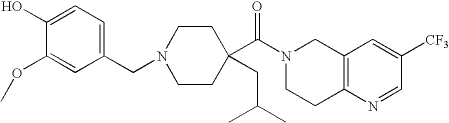 | + |
| 3 | 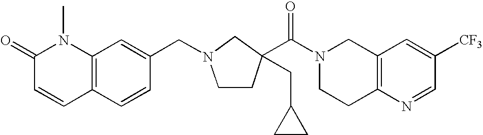 | +++ |
| 4, 5 (Enantiomer I) | 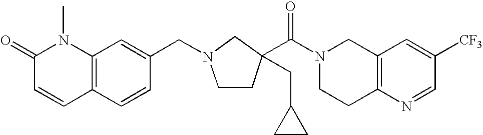 | +++ |
| 4, 5 (Enantiomer II) | 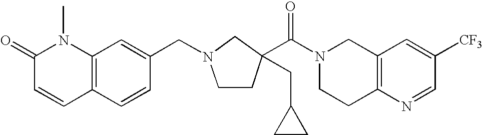 | +++ |
| 6 | 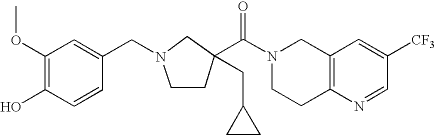 | ++ |
| 7 | 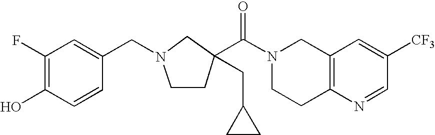 | + |
| 8 | 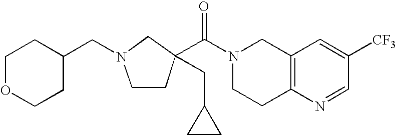 | ++ |
| 25 | 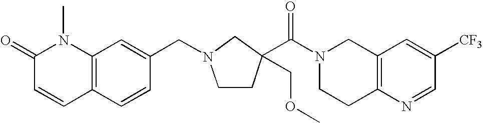 | +++ |
| 9 | 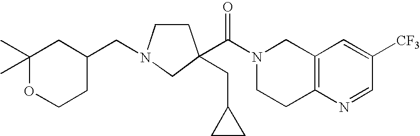 | ++ |
| 17 | 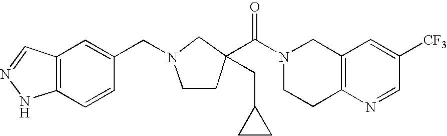 | + |
| 10 | 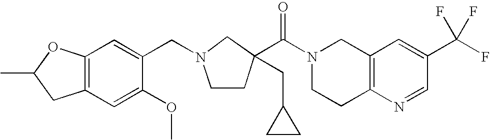 | + |
| 26, 27 (Enantiomer II) | 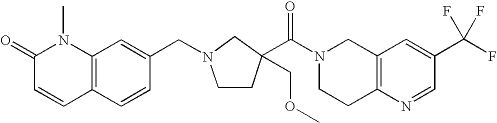 | + |
| 26, 27 (Enantiomer I) | 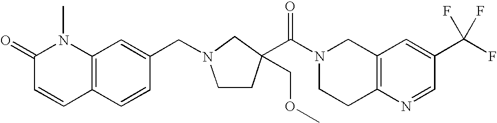 | +++ |
| 11 | 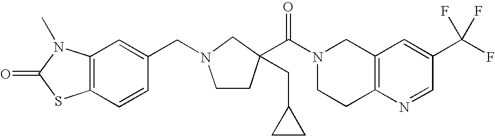 | +++ |
| 12 | 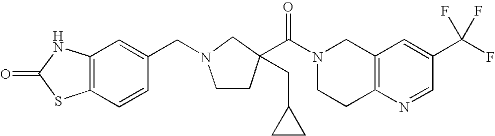 | ++ |
| 22 | 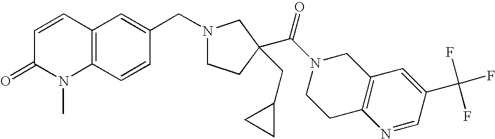 | + |
| 23 | 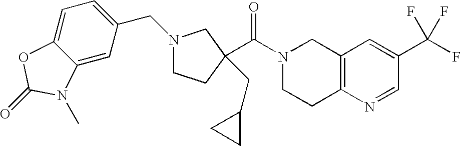 | +++ |
| 15 | 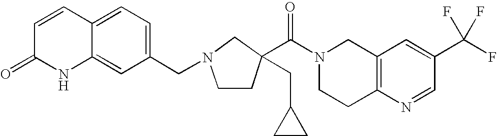 | + |
| 28 | 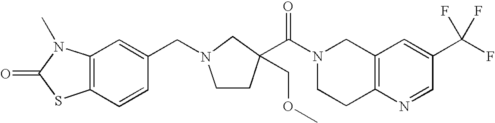 | +++ |
| 39 | 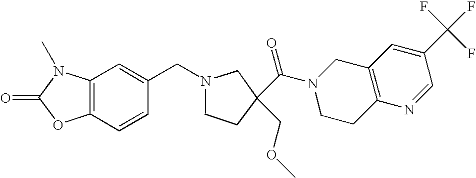 | +++ |
| 32 | 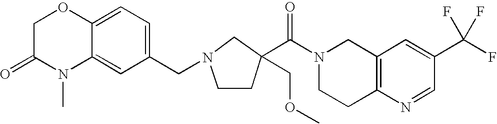 | +++ |
| 42 | 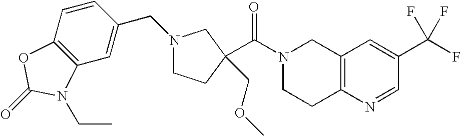 | +++ |
| 29 | 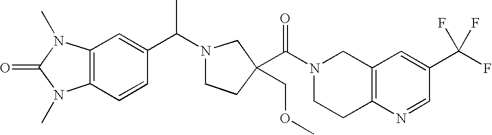 | +++ |
| 33 | 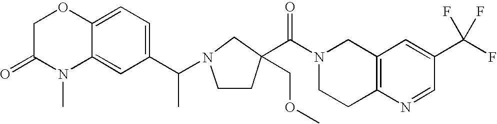 | +++ |
| 36 | 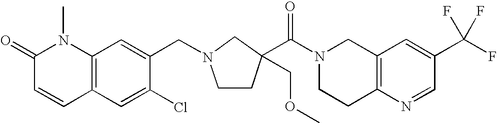 | ++ |
| 34 | 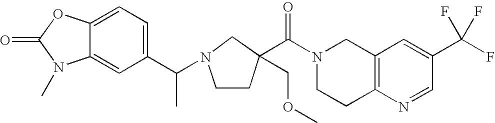 | +++ |
| 40, 41 (Enantiomer I) | 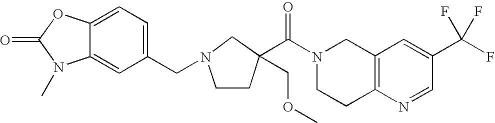 | +++ |
| 40, 41 (Enantiomer II) | 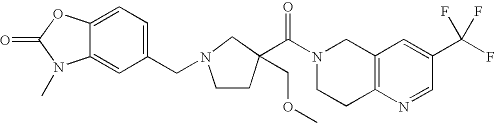 | ++ |
| 37 | 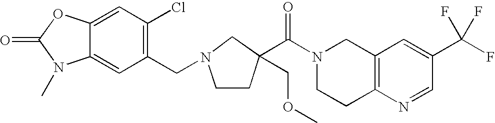 | +++ |
| 38 | 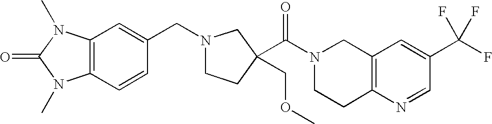 | + |
| 43 | 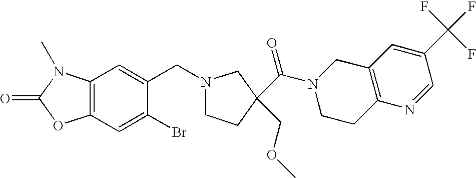 | +++ |
| 44 | 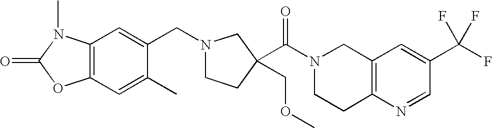 | +++ |
| 35 | 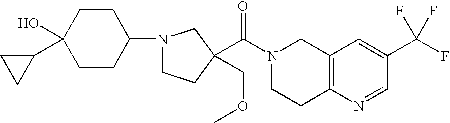 | ++ |
| 30, 31 (Isomer I) | 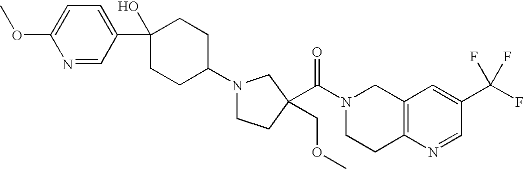 | +++ |
| 30, 31 (Isomer II) | 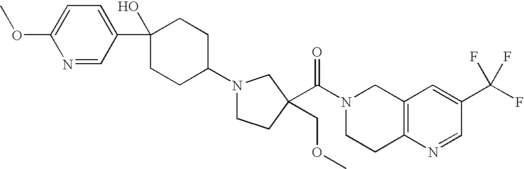 | + |
| 16 | 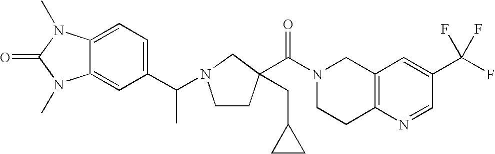 | + |
| 24 | 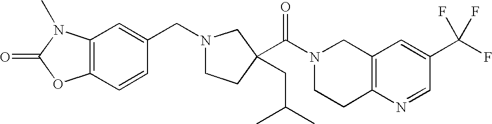 | +++ |
| 45 | 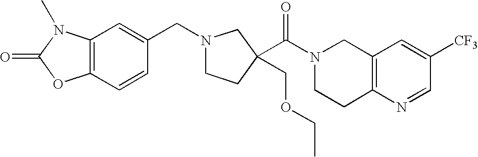 | +++ |
| 46 | 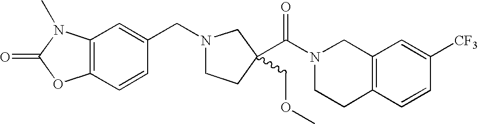 | +++ |
| 47 | 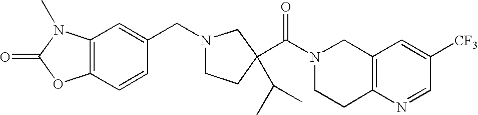 | +++ |
| 48 | 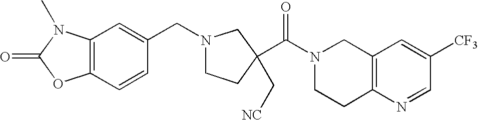 | ++ |
| 49 | 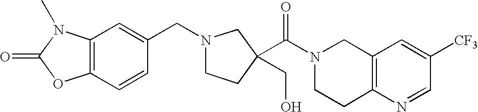 | + |
| 50, 51 (Isomer I) | 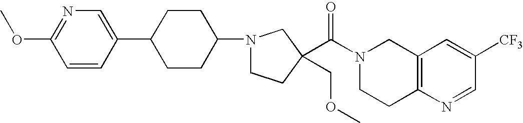 | +++ |
| 50, 51 (Isomer II) | 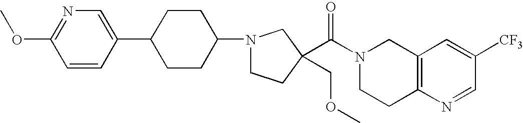 | +++ |
| 52, 53 (Isomer II) | 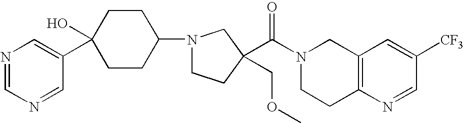 | ++ |
| 52, 53 (Isomer II) | 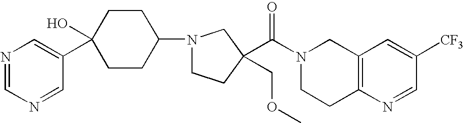 | + |
| 54, 55 (Isomer II) | 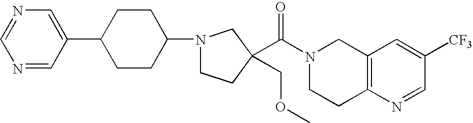 | ++ |
| 54, 55 (Isomer II) | 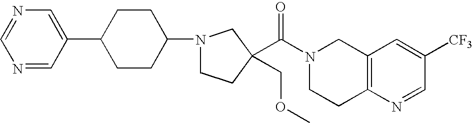 | ++ |
| 56, 57 (Isomer I) | 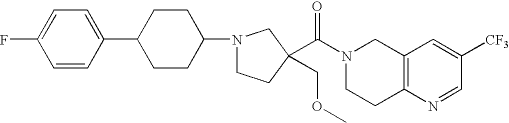 | +++ |
| 56, 57 (Isomer I) | 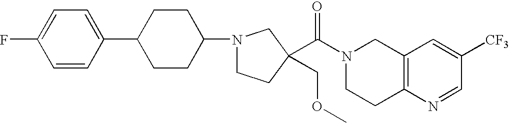 | +++ |
| 58 | 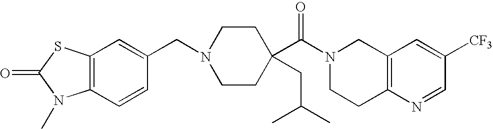 | + |

a. when IC₅₀ > 1000 nM: designated as “+”; when 1000 nM > IC₅₀ > 200 nM; designated as “++”; when IC₅₀ < 200 nM, designated as “+++”.

### EQUIVALENTS

Those skilled in the art will recognize, or be able to ascertain using no more than routine experimentation, numerous equivalents to the specific procedures described herein. Such equivalents are considered to be within the scope of the invention. Various substitutions, alterations, and modifications may be made to the invention without departing from the spirit and scope of the invention. Other aspects, advantages, and modifications are within the scope of the invention. The contents of all references, issued patents, and published patent applications cited throughout this application are hereby incorporated by reference for all purposes. The appropriate components, processes, and methods of those patents, applications and other documents may be selected for the invention and embodiments thereof.

1. A method of treating a disorder selected from the group consisting of wet age-related macular degeneration, choroidal neovascularization, diabetic retinopathy, intraocular neovascularization, retinal vein occlusion, retinopathy of prematurity, angioid streak, high myopia, macular edema, ocular histoplasmosis, retinal detachment, retinitis pigmentosa, ischemic retinopathy, iris neovascularization, corneal neovascularization, retinal neovascularization, diabetic retinal ischemia, proliferative vitreoretinopathy, uveitis, iritis, inflammatory eye disease, and dry age-related macular degeneration, comprising administering to a patient in need thereof a therapeutically effective amount of a compound of formula I-A: 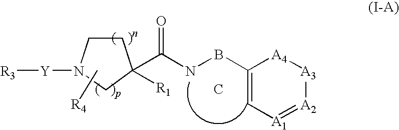
or a pharmaceutically acceptable salt thereof, wherein:
R₁ is hydrogen; alkyl, alkoxyalkyl, alkoxyphenyl, alkylthioalkyl, alkylamino, —SO₂(alkyl), C₃₋₆ cycloalkyl, C₃₋₆ heterocycloalkyl, aryl, heteroaryl, aralkyl, or heteroaralkyl, each of which is optionally substituted with 1, 2, or 3 R₅ substituents; or R₁ is optionally substituted (C₁-C₆alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 R₅ substituents;
Y is a direct bond or is CO, SO₂, —N(H)CO, —N(H)SO₂, C(═NH), C₁₋₄ alkylene, C₂₋₄ alkenylene, C₂₋₄ alkynylene, C₃₋₆ cycloalkylene, arylene, heterocycloalkylene, heteroarylene, —C(O)alkylene, —N(H)C(O)alkylene, or —O-alkylene; each of which may be optionally substituted with 1, 2, or 3 R₅ substituents;
R₃ is hydrogen; alkyl, alkenyl, alkynyl, cycloalkyl, aryl, heterocycloalkyl, heteroaryl, or —N(R₆)(R₇); each of which is optionally substituted with 1, 2, or 3 R₅ substituents; or R₃ is 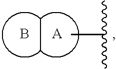
which is an optionally substituted fused aromatic or partially aromatic bicyclic or tricyclic ring, containing at least one nitrogen atom;
R₄ is hydrogen; halo; C₁₋₈ alkyl, alkenyl, or alkynyl optionally interrupted by oxygen or sulfur; cycloalkyl; alkoxy; arylalkoxy; or heteroarylalkoxy;
R₅, when present, represents independently for each occurrence hydrogen, halo, hydroxy, alkyl, alkenyl, cycloalkyl, alkoxy, —CO₂H, —CO₂C₁₋₃alkyl, cyano, aryl, heteroaryl, aralkyl, heteroaralkyl, oxo, —CF₃, —O—CF₃, —O—CHF₂, —O—CH₂F, —O-aryl, —N(H)alkyl, —N(H)SO₂-alkyl, —N(H)C(O)alkyl, —SO₂N(H)alkyl, —SO₂N(alkyl)C(O)alkyl, or —C(O)N(H)SO₂alkyl;
R₆ is hydrogen, alkyl, cycloalkyl, heterocycloalkyl, aryl, or heteroaryl;
R₇ is hydrogen or C₁₋₃ alkyl;
n is 0, 1, 2, or 3;
p is 1 or 2;
A₁, A₂, A₃, and A₄ are independently N or C—R₅, provided that at least two of A₁, A₂, A₃, or A₄ are C—R₅; and 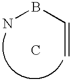
is an optionally substituted 5, 6, or 7-membered mono- or bicyclic ring optionally containing a heteroatom selected from the group consisting of O, S, SO, SO₂, N—H, N-alkyl, and N—CO-alkyl, in which B is C₁-C₂alkylene or C₂-C₄alkenylene, and in which the ring is optionally substituted with 1 or 2 halo, methyl, or ethyl groups, or is geminally substituted to form a cyclopropyl ring.

2. The method of claim 1, wherein R₁ is hydrogen; alkyl, alkoxyalkyl, alkoxy-CHF₂, alkoxy-CH₂F, alkoxy-CF₃, C₃₋₆ cycloalkyl, C₃₋₆ heterocycloalkyl, aryl, heteroaryl, or (C₁-C₆alkylene)-R₁ₐ; wherein R₁ₐ is C₃₋₆ cycloalkyl, C₃₋₆ heterocycloalkyl, aryl, or heteroaryl, each of which may be independently optionally substituted with 1, 2, or 3 R₅ substituents.

3. The method of claim 1, wherein R₁ alkoxyalkyl.

4. The method of claim 1, wherein R₁ is —CH₂—O—CH₃, —CH₂—O—CF₃, —CH₂—O—CHF₂, —CH₂—O—CH₂F, —CH₂—O—CH₂—CH₃, —CH₂—O—CH—(CH₃)₂, or —CH₂—CN.

5. The method of claim 1, wherein Y is CH₂, 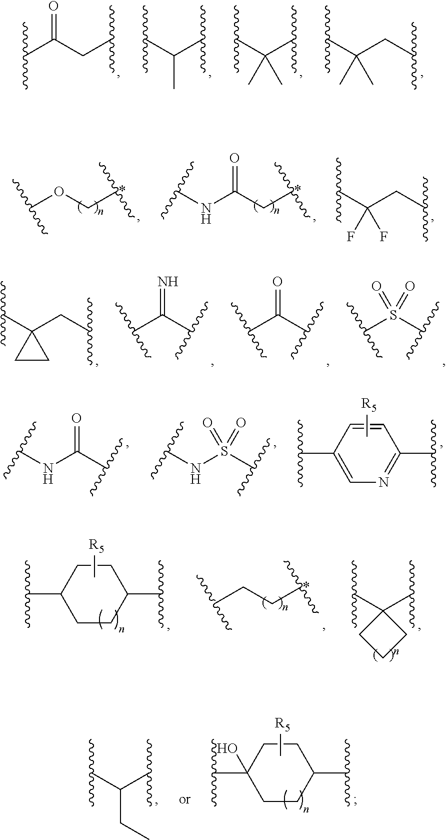
wherein n is 0, 1 or 2.

6. The method of claim 1, wherein Y is CH₂.

7. The method of claim 1, wherein R₃ is

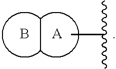

8. The method of claim 7, wherein 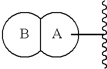
is represented by 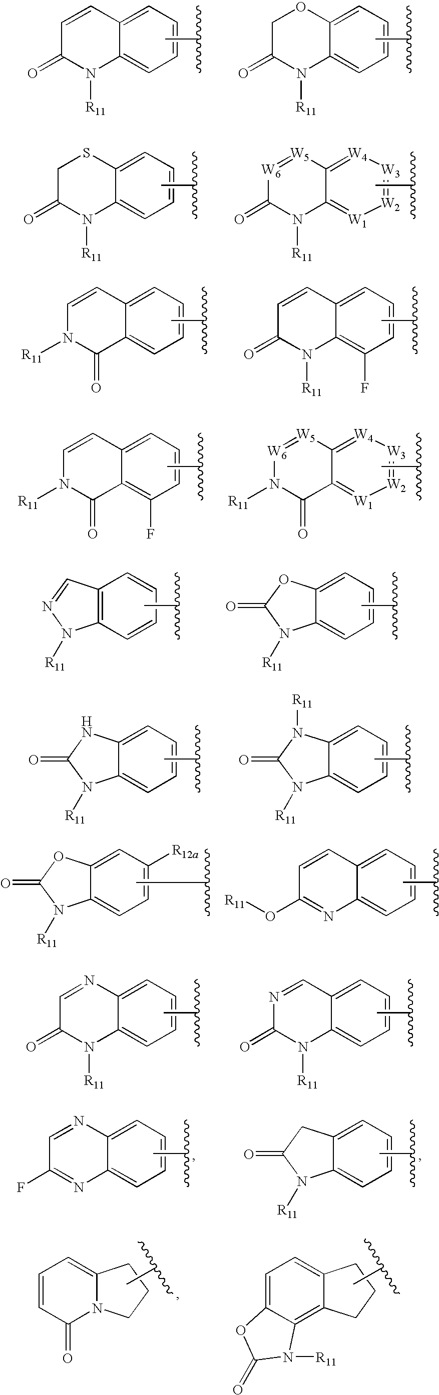 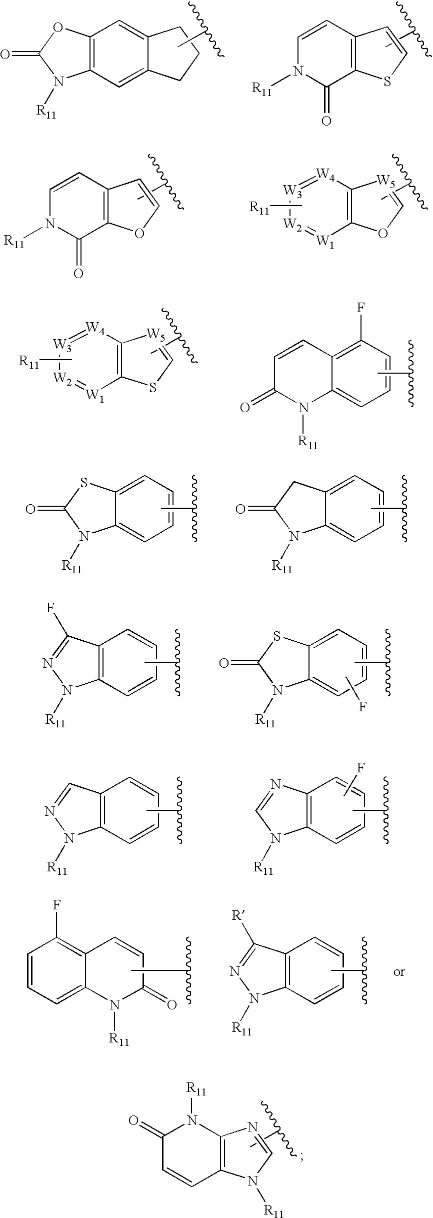
wherein W₁, W₂, W₃, W₄, W₅, and W₆ are independently C, N, C═O, C—OH, C—OR₁₀ or C—R₁₀;
R₁₀ is hydrogen, C₁₋₆ alkyl, C₁₋₅ alkylthio, C₁₋₅ alkoxy, halogen, hydroxyl, cyano, halogen-substituted C₁₋₆ alkyl, or halogen-substituted C₁₋₅ alkoxy;
R₁₁, independently for each occurrence, is hydrogen or is C₁₋₆ alkyl, (C₁-C₆alkylene)cycloalkyl, aralkyl, or heteroaralkyl, any of which may be optionally substituted with halo, hydroxy, alkyl, alkenyl, cycloalkyl, C₁₋₃alkoxy, —CO₂H, —CO₂C₁₋₃alkyl, cyano, aryl, heteroaryl, —CF₃, —O—CF₃, —O—CH₂F, or —O—CHF₂;
R₁₂ₐ is H, halo, alkyl, or alkoxy; and
R′ is alkyl, haloalkyl, or cycloalkyl.

9. The method of claim 7, wherein 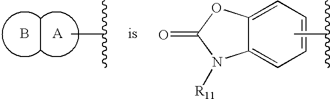
wherein R₁₁ is hydrogen, methyl, ethyl, or propyl.

10. The method of claim 1, wherein 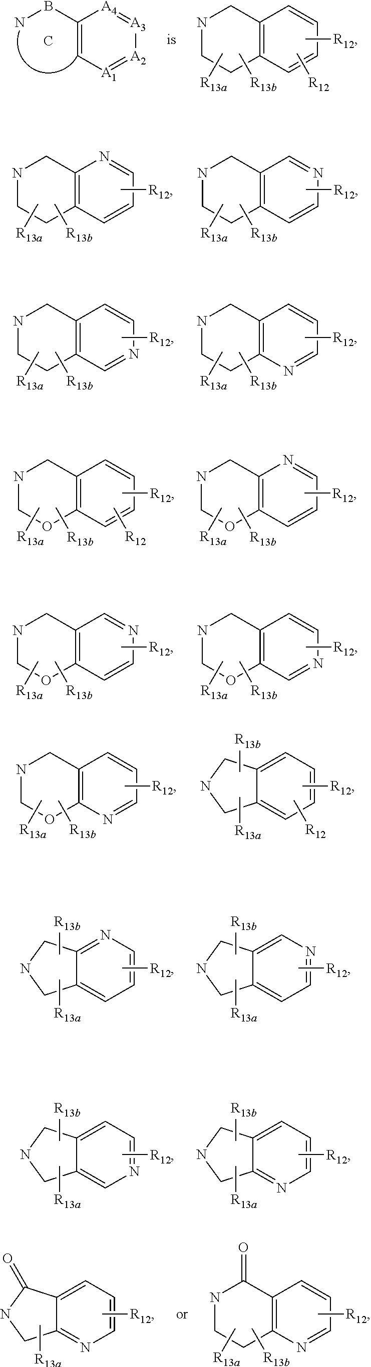
wherein R₁₂ is, independently for each occurrence, hydrogen, halo, alkyl, haloalkyl, haloalkoxy, alkoxy, or cyano; and
R₁₃ₐ and R_{13b} are each independently hydrogen, halo, alkyl, haloalkyl, alkoxy, haloalkoxy, or if R₁₃ₐ and R_{13b} are attached to the same carbon, they can form C═O when taken together with the carbon to which they are attached.

11. The method of claim 1, wherein 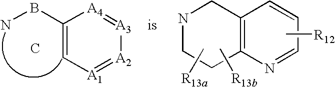
and R₁₂, R₁₃ₐ, and R_{13b} are each independently hydrogen, halo, alkyl, or haloalkyl.

12. The method of claim 1, wherein R₄ is hydrogen.

13. The method of claim 1, wherein n and p are 1.

14. The method of claim 1, wherein the compound is a compound of formula I-B2: 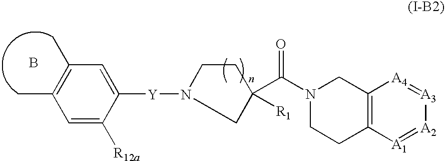
or a pharmaceutically acceptable salt thereof, wherein:
A₁, A₂, A₃, and A₄ are independently N or C—R₅, provided that at least two of A₁, A₂, A₃, or A₄ are C—R₅;
R₁ is alkoxyalkyl;
R₅ represents independently for each occurrence hydrogen, halo, hydroxy, alkyl, alkenyl, cycloalkyl, alkoxy, cyano, or —CF₃;
R₁₂ₐ is hydrogen, halo, alkyl, or alkoxy;
n is 1 or 2;
Y is C₁-C₃ alkylene; and 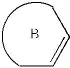
is an unsaturated heterocyclic ring optionally substituted with 1 or 2 groups selected from the group consisting of halo, alkyl, and oxo.

15. The method of claim 14, wherein A₁ is N or C—R₅; and A₂, A₃, and A₄ are C—R₅.

16. The method of claim 1, wherein the compound is a compound of formula I-C1: 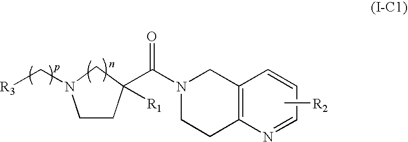
or a pharmaceutically acceptable salt thereof, wherein:
R₁ is alkoxyalkyl;
R₂ is alkyl, haloalkyl, halogen or alkoxy;
R₃ is 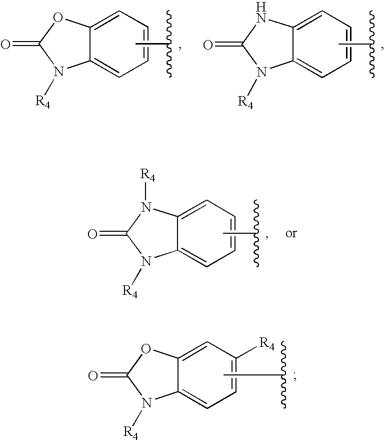
R₄ represents independently for each occurrence hydrogen or alkyl; and
n and p each represent independently 1 or 2.

17. The method of claim 16, wherein R₁ is —(CH₂)ₓ—O—(CH₂)ₓ—H, wherein X represents independently 1, 2, or 3.

18. The method of claim 16, wherein R₂ is haloalkyl

19. The method of claim 16, wherein R₂ is —CF₃.

20. The method of claim 16, wherein R₃ is

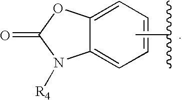

21. The method of claim 16, wherein R₄ represents independently methyl, ethyl, or propyl.

22. The method of claim 16, wherein n and p are 1.

23. The method of claim 1, wherein the compound is:
(1-(4-hydroxy-3-methoxybenzyl)-4-isobutylpiperidin-4-yl)(3-(trifluoromethyl)-7,8-dihydro-1,6-naphthyridin-6(5H)-yl)methanone; 7-((4-isobutyl-4-(3-(trifluoromethyl)-5,6,7,8-tetrahydro-1,6-naphthyridine-6-carbonyl)piperidin-1-yl)methyl)-1-methylquinolin-2(1H)-one; (3-(cyclopropylmethyl)-1-(4-hydroxy-3-methoxybenzyl)pyrrolidin-3-yl)(3-(trifluoromethyl)-7,8-dihydro-1,6-naphthyridin-6(5H)-yl)methanone; 7-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (S)-7-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (R)-7-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (3-(cyclopropylmethyl)-1-(3-fluoro-4-hydroxybenzyl)pyrrolidin-3-yl)(3-(trifluoromethyl)-7,8-dihydro-1,6-naphthyridin-6(5H)-yl)methanone; (3-(cyclopropylmethyl)-1-((tetrahydro-2H-pyran-4-yl)methyl)pyrrolidin-3-yl)(3-(trifluoromethyl)-7,8-dihydro-1,6-naphthyridin-6(5H)-yl)methanone; or a pharmaceutically acceptable salt thereof.

24. The method of claim 1, wherein the compound is:
(3-(cyclopropylmethyl)-1-((2,2-dimethyltetrahydro-2H-pyran-4-yl)methyl)pyrrolidin-3-yl)(3-(trifluoromethyl)-7,8-dihydro-1,6-naphthyridin-6(5H)-yl)methanone; (3-(cyclopropylmethyl)-1-(5-methoxy-2-methyl-2,3-dihydrobenzofuran-6-yl)methyl)pyrrolidin-3-yl)(3-(trifluoromethyl)-7,8-dihydro-1,6-naphthyridin-6(5H)-yl)methanone; 5-((3-(cyclopropylmethyl)-3-(3-(trifluoromethyl)-5,6,7,8-tetrahydro-1,6-naphthyridine-6-carbonyl)pyrrolidin-1-yl)methyl)-3-methylbenzo[d]thiazol-2(3H)-one; 5-((3-(cyclopropylmethyl)-3-(3-(trifluoromethyl)-5,6,7,8-tetrahydro-1,6-naphthyridine-6-carbonyl)pyrrolidin-1-yl)methyl)benzo[d]thiazol-2(3H)-one; 3-(cyclopropylmethyl)-1-(4-hydroxy-4-(6-methoxypyridin-3-yl)cyclohexyl)pyrrolidin-3-yl)(3-(trifluoromethyl)-7,8-dihydro-1,6-naphthyridin-6(5H)-yl)methanone; (3-(cyclopropylmethyl)-1-(2,2-dimethylchromoa-6-yl)methyl)pyrrolidin-3-yl)(3-(trifluoromethyl)-7,8-dihydro-1,6-naphthyridin-6(5H)-yl)methanone; 7-((3-(cyclopropylmethyl)-3-(3-(trifluoromethyl)-5,6,7,8-tetrahydro-1,6-naphthyridin-6-carbonyl)pyrrolidin-1-yl)methyl)quinolin-2(1H)-one; 5-(1-(3-(cyclopropylmethyl)-3-(3-(trifluoromethyl)-5,6,7,8-tetrahydro-1,6-naphthyridine-6-carbonyl)pyrrolidin-1-yl)ethyl)-1,3-dimethyl-1H-benzo[d]imidazol-2(3H)-one; (1-((1H-indazol-5-yl)methyl)-3-(cyclopropylmethyl)pyrrolidin-3-yl)(3-(trifluoromethyl)-7,8-dihydro-1,6-naphthyridin-6(5H)-yl)methanone; (3-(cyclopropylmethyl)-1-(4-hydroxy-4-(6-methoxypyridin-3-yl)cyclohexyl)pyrrolidin-3-yl)(3-(trifluoromethyl)-7,8-dihydro-1,6-naphthyridin-6(5H)-yl)methanone; 6-((3-(cyclobutylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 6-((3-isopentyl-3-(3-(trifluoromethyl)-5,6,7,8-tetrahydro-1,6-naphthyridine-6-carbonyl)pyrrolidin-1-yl)methyl)-1-methylquinolin-2(1H)-one; 6-((3-benzyl-3-(3-(trifluoromethyl)-5,6,7,8-tetrahydro-1,6-naphthyridine-6-carbonyl)pyrrolidin-1-yl)methyl)-1-methylquinolin-2(1H)-one; 6-((3-(cyclopropylmethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 5-((3-(cyclopropylmethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 5-((3-isobutyl-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (S)-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; (R)-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 5-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]thiazol-2(3H)-one; 5-(1-(3-(methoxymethyl)-3-(3-(trifluoromethyl)-5,6,7,8-tetrahydro-1,6-naphthyridine-6-carbonyl)pyrrolidin-1-yl)ethyl)-1,3-dimethyl-1H-benzo[d]imidazol-2(3H)-one; (1-(4-hydroxy-4-(6-methoxypyridin-3-yl)cyclohexyl)-3-(methoxymethyl)pyrrolidin-3-yl)(3-(trifluoromethyl)-7,8-dihydro-1,6-naphthyridin-6(5H)-yl)methanone; 6-((3-(methoxymethyl)-3-(3-(trifluoromethyl)-5,6,7,8-tetrahydro-1,6-naphthyridine-6-carbonyl)pyrrolidin-1-yl)methyl)-4-methyl-2H-benzo[b][1,4]oxazin-3(4H)-one; 6-(1-(3-(methoxymethyl)-3-(3-(trifluoromethyl)-5,6,7,8-tetrahydro-1,6-naphthyridine-6-carbonyl)pyrrolidin-1-yl)ethyl)-4-methyl-2H-benzo[b][1,4]oxazin-3(4H)-one; 5-(1-(3-(methoxymethyl)-3-(3-(trifluoromethyl)-5,6,7,8-tetrahydro-1,6-naphthyridine-6-carbonyl)pyrrolidin-1-yl)ethyl)-3-methylbenzo[d]oxazol-2(3H)-one; (1-(4-cyclopropyl-4-hydroxycyclohexyl)-3-(methoxymethyl)pyrrolidin-3-yl)(3-trifluoromethyl)-7,8-dihydro-1,6-naphthyridin-6(5H)-yl)methanone; 6-chloro-7-((3-(methoxymethyl)-3-(3-(trifluoromethyl)-5,6,7,8-tetrahydro-1,6-naphthyridine-6-carbonyl)pyrrolidin-1-yl)methyl)-1-methylquinolin-2(1H)-one; 6-chloro-5-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 5-((3-(methoxymethyl)-3-(3-(trifluoromethyl)-5,6,7,8-tetrahydro-1,6-naphthyridine-6-carbonyl)pyrrolidin-1-yl)methyl)-1,3-dimethyl-1H-benzo[d]imidazol-2(3H)-one; 5-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; (R)-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one and (S)-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 3-ethyl-6-((3-(methoxymethyl)-3-(3-(trifluoromethyl)-5,6,7,8-tetrahydro-1,6-naphthyridine-6-carbonyl)pyrrolidin-1-yl)methyl)benzo[d]oxazol-2(3H)-one; 5-bromo-6-((3-(methoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 6-((3-(methoxymethyl)-3-(3-(trifluoromethyl)-5,6,7,8-tetrahydro-1,6-naphthyridine-6-carbonyl)pyrrolidin-1-yl)methyl)-3,5-dimethylbenzo[d]oxazol-2(3H)-one; 5-((3-(ethoxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 5-((3-(methoxymethyl)-3-(7-(trifluoromethyl)-1,2,3,4-tetrahydroisoquinoline-2-carbonyl)pyrrolidin-1-yl)methyl)-3-methylbenzo[d]oxazol-2(3H)-one; 5-((3-isopropyl-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; 2-(1-((3-methyl-2-oxo-2,3-dihydrobenzo[d]oxazol-5-yl)methyl)-3-(3-(trifluoromethyl)-5,6,7,8-tetrahydro-1,6-naphthyridine-6-carbonyl)pyrrolidin-3-yl)acetonitrile; 5-((3-(hydroxymethyl)-3-(3-(trifluoromethyl)-5,6,7,8-tetrahydro-1,6-naphthyridine-6-carbonyl)pyrrolidin-1-yl)methyl)-3-methylbenzo[d]oxazol-2(3H)-one; (3-(methoxymethyl)-1-(4-(6-methoxypyridin-3-yl)cyclohexyl)pyrrolidin-3-yl)(3-(trifluoromethyl)-7,8-dihydro-1,6-naphthyridin-6(5H)-yl)methanone; (1-(4-hydroxy-4-(pyrimidin-5-yl)cyclohexyl)-3-(methoxymethyl)pyrrolidin-3-yl)(3-(trifluoromethyl)-7,8-dihydro-1,6-naphthyridin-6(5H)-yl)methanone; (3-(methoxymethyl)-1-(4-(pyrimidin-5-yl)cyclohexyl)pyrrolidin-3-yl)(3-(trifluoromethyl)-7,8-dihydro-1,6-naphthyridin-6(5H)-yl)methanone; (1-(4-(4-fluorophenyl)cyclohexyl)-3-(methoxymethyl)pyrrolidin-3-yl)(3-(trifluoromethyl)-7,8-dihydro-1,6-naphthyridin-6(5H)-yl)methanone; 6-((4-isobutyl-4-(3-(trifluoromethyl)-5,6,7,8-tetrahydro-1,6-naphthyridine-6-carbonyl)piperidin-1-yl)methyl)-3-methylbenzo[d]thiazol-2(3H)-one; 6-((4-isobutyl-4-(3-(trifluoromethyl)-5,6,7,8-tetrahydro-1,6-naphthyridine-6-carbonyl)piperidin-1-yl)methyl)-3-methylbenzo[d]oxazol-2(3H)-one; 2-(3-(methoxymethyl)-1-((3-methyl-2-oxo-2,3-dihydrobenzo[d]oxazol-5-yl)methyl)pyrrolidine-3-carbonyl)-7-(trifluoromethyl)-1,2,3,4-tetrahydroisoquinoline-5-carbonitrile; 5-((3-(methoxymethyl)-3-(7-(trifluoromethyl)-1,2,3,4-tetrahydroisoquinoline-2-carbonyl)pyrrolidin-1-yl)methyl)-3-methylbenzo[d]oxazol-2(3H)-one; 6-(3-(methoxymethyl)-1-((3-methyl-2-oxo-2,3-dihydrobenzo[d]oxazol-5-yl)methyl)pyrrolidine-3-carbonyl)-5,6,7,8-tetrahydro-1,6-naphthyridine-3-carbonitrile; 5-((3-(methoxymethyl)-3-(1,2,3,4-tetrahydroisoquinoline-2-carbonyl)pyrrolidin-1-yl)methyl)-3-methylbenzo[d]oxazol-2(3H)-one; 5-((3-(methoxymethyl)-3-(3-(trifluoromethyl)-6,7-dihydro-5H-pyrrolo[3,4-b]pyridine-6-carbonyl)pyrrolidin-1-yl)methyl)-3-methylbenzo[d]oxazol-2(3H)-one; 5-(2-(3-(methoxymethyl)-3-(3-(trifluoromethyl)-5,6,7,8-tetrahydro-1,6-naphthyridine-6-carbonyl)pyrrolidin-1-yl)propan-2-yl)-3-methylbenzo[d]oxazol-2(3H)-one; 5-(1-(3-(methoxymethyl)-3-(3-(trifluoromethyl)-5,6,7,8-tetrahydro-1,6-naphthyridine-6-carbonyl)pyrrolidin-1-yl)cyclopropyl)-3-methylbenzo[d]oxazol-2(3H)-one; 6-(1-(3-(ethoxymethyl)-3-(3-(trifluoromethyl)-5,6,7,8-tetrahydro-1,6-naphthyridine-6-carbonyl)pyrrolidin-1-yl)ethyl)-4-methyl-2H-benzo[b][1,4]oxazin-3(4H)-one; 74(3-(methoxymethyl)-3-(3-(trifluoromethyl)-5,6,7,8-tetrahydro-1,6-naphthyridine-6-carbonyl)pyrrolidin-1-yl)methyl)-1-methylquinoxalin-2(1H)-one; 7-(1-(3-(methoxymethyl)-3-(3-(trifluoromethyl)-5,6,7,8-tetrahydro-1,6-naphthyridine-6-carbonyl)pyrrolidin-1-yl)ethyl)-1-methylquinoxalin-2(1H)-one; 5-(3-(methoxymethyl)-3-(3-(trifluoromethyl)-5,6,7,8-tetrahydro-1,6-naphthyridine-6-carbonyl)pyrrolidin-1-yl)-3-methyl-3,5,6,7-tetrahydro-2H-indeno[5,6-d]oxazol-2-one; 6-(3-(methoxymethyl)-3-(3-(trifluoromethyl)-5,6,7,8-tetrahydro-1,6-naphthyridine-6-carbonyl)pyrrolidin-1-yl)-1-methyl-7,8-dihydro-1H-indeno[4,5-d]oxazol-2(6H)-one; 6-((3-(methoxymethyl)-3-(3-(trifluoromethyl)-5,6,7,8-tetrahydro-1,6-naphthyridine-6-carbonyl)pyrrolidin-1-yl)methyl)-1-methylindolin-2-one; 6-(1-(3-(methoxymethyl)-3-(3-(trifluoromethyl)-5,6,7,8-tetrahydro-1,6-naphthyridine-6-carbonyl)pyrrolidin-1-yl)ethyl)-1-methylindolin-2-one; (1-(4-(5-fluoropyridin-2-yl)cyclohexyl)-3-(methoxymethyl)pyrrolidin-3-yl)(3-(trifluoromethyl)-7,8-dihydro-1,6-naphthyridin-6(5H)-yl)methanone; (1-(4-fluoro-4-(6-methoxypyridin-3-yl)cyclohexyl)-3-(methoxymethyl)pyrrolidin-3-yl)(3-(trifluoromethyl)-7,8-dihydro-1,6-naphthyridin-6(5H)-yl)methanone; (3-(methoxymethyl)-1-(4-(pyrimidin-2-yl)cyclohexyl)pyrrolidin-3-yl)(3-(trifluoromethyl)-7,8-dihydro-1,6-naphthyridin-6(5H)-yl)methanone; or a pharmaceutically acceptable salt thereof.

25. The method of claim 1, wherein the compound is: 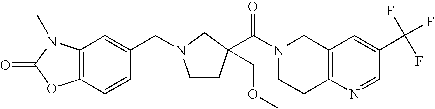
or a pharmaceutically acceptable salt thereof.

26. The method of claim 1, wherein the compound is:

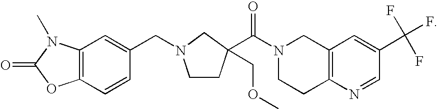

27. The method of claim 1, wherein the disorder is wet age-related macular degeneration.

28. The method of claim 1, wherein the disorder is choroidal neovascularization or diabetic retinopathy.

29. The method of claim 1, wherein the patient is a human.
